(19) United States
(12) Patent Application Publication
(10) Pub. No.: US 2010/0261706 A1
(43) Pub. Date: Oct. 14, 2010

(54) SUBSTITUTED TETRACYCLIC 1H-INDENO [1,2-B]PYRIDINE-2(5H)-ONE ANALOGS THEREOF AND USES THEREOF

(75) Inventors: Prakash JAGTAP, N. Andover, MA (US); Duy-Phong Pham-Huu, Beverly, MA (US); Frederick COHEN, San Francisco, CA (US); Huiyong HU, Belmont, CA (US); Xiaojing WANG, Foster City, CA (US)

Correspondence Address: LAHIVE & COCKFIELD, LLP;FLOOR 30, SUITE 3000, ONE POST OFFICE SQUARE, BOSTON, MA, 02109, US

(73) Assignee: INOTEK PHARMACEUTICALS CORPORATION, Lexington, MA (US)

(21) Appl. No.: 12/633,379

(22) Filed: Dec. 8, 2009

Related U.S. Application Data
(60) Provisional application No. 61/201,281, filed on Dec. 8, 2008.

(51) Int. Cl. A61K 31/55; C07D 221/18; A61K 31/473; C07D 401/10; A61K 31/497; C07D 413/10; A61K 31/5377; C07D 471/04; A61K 31/4743; C07D 279/12; A61K 31/541; A61P 27/02; A61P 25/00; A61P 13/12; A61P 9/00; A61P 13/00; A61P 15/10; A61P 3/10; A61P 29/00; A61P 35/00

(52) U.S. Cl. 514/217.07; 546/61; 514/284; 540/597; 540/481; 544/361; 514/253.02; 544/125; 514/232.8; 546/62; 514/285; 546/70; 544/58.2; 514/228.2

(57) ABSTRACT

Provided herein are tetracyclic 1H-indeno[1,2-b]pyridine-2(5H)-one analogs and derivatives, compositions comprising an effective amount of a tetracyclic 1H-indeno[1,2-b]pyridine-2(5H)-one analog and/or derivative and methods for treating or preventing an inflammatory disease, a reperfusion injury, diabetes mellitus, a diabetic complication, a reoxygenation injury resulting from organ transplantation, an ischemic condition, a neurodegenerative disease, renal failure, a vascular disease, a cardiovascular disease, an ocular or opthalmologic disease, cancer, a complication of prematurity, cardiomyopathy, retinopathy, nephropathy, contrast induced nephropathy, neuropathy, erectile dysfunction or urinary incontinence, comprising administering to a subject in need thereof an effective amount of a tetracyclic 1H-indeno[1,2-b]pyridine-2(5H)-one analog or derivative.

### 1. RELATED APPLICATION

This application claims priority to U.S. Provisional Application No. 61/201,281, Attorney Docket No. ITJ-040-1, filed Dec. 8, 2008. The contents of any patents, patent applications, and references cited throughout this specification are hereby incorporated by reference in their entireties.

### 2. FIELD OF THE INVENTION

Provided herein are tetracyclic 1H-indeno[1,2-b]pyridine-2(5H)-one analogs and derivatives, compositions comprising an effective amount of a tetracyclic 1H-indeno[1,2-b]pyridine-2(5H)-one analog and/or derivative and methods for treating or preventing an inflammatory disease, a reperfusion injury, diabetes mellitus, a diabetic complication, a reoxygenation injury resulting from organ transplantation, an ischemic condition, a neurodegenerative disease, renal failure, a vascular disease, a cardiovascular disease, an ocular or opthalmologic disease, cancer, a complication of prematurity, cardiomyopathy, retinopathy, nephropathy, contrast induced nephropathy, neuropathy, erectile dysfunction or urinary incontinence, comprising administering to a subject in need thereof an effective amount of a tetracyclic 1H-indeno[1,2-b]pyridine-2(5H)-one analog or derivative.

### 3. BACKGROUND OF THE INVENTION

An inflammatory disease, such as arthritis, colitis, uveitis and autoimmune diabetes, typically manifests itself as a disorder distinct from that associated with a reperfusion injury, e.g., stroke and heart attack, and can clinically manifest itself as a different entity. However, there can be common underlying mechanisms between these two types of disorders. Specifically, inflammatory disease and reperfusion injury can induce proinflammatory cytokine and chemokine synthesis which can, in turn, result in production of cytotoxic free radicals such as nitric oxide and superoxide. NO and superoxide can react to form peroxynitrite (ONOO⁻)(Szabó et al., Shock 6:79-88, 1996).

Erectile dysfunction (“ED”) is a significant male-health issue. While estimating its prevalence is difficult, estimates range from about 15 million to 30 million sufferers worldwide.

The etiology of erectile dysfunction can be multiple, and can include mechanical trauma to the nerves (such as during prostatectomy), or it can be due to diabetes mellitus, cardiovascular diseases, induced by radiation, certain drugs, or advanced age.

Urinary incontinence affects people of all ages and levels of physical health, both in health care settings and in the community at large. Persons suffering from urinary incontinence can be predisposed to also having urinary-tract infections, pressure ulcers, perineal rashes and urosepsis. Psychosocially, urinary incontinence can be associated with embarrassment, social stigmatization, depression and a risk of institutionalization (Herzo et al., Annu. Rev. Gerontol. Geriatr. 9:74 (1989)). The ONOO⁻-induced cell necrosis observed in inflammatory disease and in reperfusion injury involves the activation of the nuclear enzyme poly (ADP-ribose) polymerase (PARP). Activation of PARP is thought to be an important step in the cell-mediated death observed in inflammation and reperfusion injury (Szabó et al., Trends Pharmacol. Sci. 19:287-98, 1998).

A number of PARP inhibitors have been described in the art. See, e.g., Banasik et al., J. Biol. Chem., 267:1569-75, 1992, and Banasik et al., Mol. Cell. Biochem., 138:185-97, 1994; WO 00/39104; WO 00/39070; WO 99/59975; WO 99/59973; WO 99/11649; WO 99/11645; WO 99/11644; WO 99/11628; WO 99/11623; WO 99/11311; WO 00/42040; Zhang et al., Biochem. Biophys. Res. Commun., 278:590-98, 2000; White et al., J. Med. Chem., 43:4084-4097, 2000; Griffin et al., J. Med. Chem., 41:5247-5256, 1998; Shinkwin et al., Bioorg. Med. Chem., 7:297-308, 1999; Jagtap et al, Bioorg. & Med. Chem. Lett., 14: 81-84, 2004; Tikhe et al, J. Med. Chem., 47: 5467-5481, 2004; Hattori et al., J. Med. Chem., 47: 4151-4154, 2004; Ferraris et al., J. Med. Chem., 46: 3138-3151, 2003; Skalitzky et al, J. Med. Chem., 46: 210-213, 2003; and Soriano et al., Nature Medicine, 7:108-113, 2001. Adverse effects associated with administration of PARP inhibitors have been discussed in Milan et al., Science, 223:589-591, 1984.

Indenoisoquinolinone compounds have been previously discussed in the art. For example, cytotoxic non-camptothecin topoisomerase I inhibitors are reported in Cushman et al., J. Med. Chem., 43:3688-3698, 2300 and Cushman et al., J. Med. Chem. 42:446-57, 1999; indeno[1,2-c]isoquinolines are reported as antineoplastic agents in Cushman et al., WO 00/21537; and as neoplasm inhibitors in Hrbata et al., WO 93/05023.

Syntheses of indenoisoquinolinone compounds have been reported. For example, see Wawzonek et al., Org. Prep. Proc. Int., 14:163-8, 1982; Wawzonek et al., Can. J. Chem., 59:2833, 1981; Andoi et al., Bull. Chem. Soc. Japan, 47:1014-17, 1974; Dusemund et al., Arch. Pharm (Weinheim, Ger.), 3 17:381-2, 1984; Lal et al., Indian J. Chem., Sect. B, 38B:33-39, 1999; and Jagtap et al., Org. Lett., 7: 1753-1756, 2005.

Citation of any reference in this specification is not an admission that the reference is prior art.

Accordingly, there remains a need for treatment of the aforementioned diseases and disorders, including diseases and disorders associated with exposure to a reactive species.

### 4. SUMMARY OF THE INVENTION

In one aspect the invention provides a compound of Formula (I)

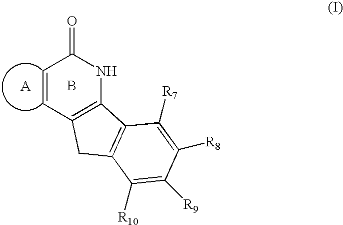

and pharmaceutically acceptable salts thereof.

The invention provides compositions comprising an effective amount of a compound of Formula (I), or a pharmaceutically acceptable salt thereof and a physiologically acceptable carrier or vehicle.

The invention further provides methods for treating or preventing an inflammatory disease, a reperfusion injury, diabetes mellitus, a diabetic complication, reoxygenation injury resulting from organ transplantation, an ischemic condition, a neurodegenerative disease, renal failure, a vascular disease, a cardiovascular disease, cancer, an ocular or opthalmologic disease, a complication of prematurity, cardiomyopathy, retinopathy, nephropathy, contrast induced nephropathy, neuropathy, erectile dysfunction or urinary incontinence (each being a “Condition”), comprising administering to a subject in need thereof an effective amount of a tetracyclic 1H-indeno[1,2-b]pyridine-2(5H)-one analog of Formula (I), or a pharmaceutically acceptable salt thereof.

The invention further provides for the use of one or more compounds of Formula (I) or a pharmaceutically acceptable salt thereof defined above in the treatment or prevention of an inflammatory disease, a reperfusion injury, diabetes mellitus, a diabetic complication, reoxygenation injury resulting from organ transplantation, an ischemic condition, a neurodegenerative disease, renal failure, a vascular disease, a cardiovascular disease, cancer, an ocular or opthalmologic disease, a complication of prematurity, cardiomyopathy, retinopathy, nephropathy, contrast induced nephropathy, neuropathy, erectile dysfunction or urinary incontinence in a subject.

The invention further provides for the use of one or more compounds of Formula (I) or a pharmaceutically acceptable salt thereof defined above in an effective amount in the manufacture of a medicament for administering to a subject in need of treatment or prevention of an inflammatory disease, a reperfusion injury, diabetes mellitus, a diabetic complication, reoxygenation injury resulting from organ transplantation, an ischemic condition, a neurodegenerative disease, renal failure, a vascular disease, a cardiovascular disease, cancer, an ocular or opthalmologic disease, a complication of prematurity, cardiomyopathy, retinopathy, nephropathy, contrast induced nephropathy, neuropathy, erectile dysfunction or urinary incontinence.

A compound of Formula (I), or a pharmaceutically acceptable salt thereof (a “tetracyclic 1H-indeno[1,2-b]pyridine-2(5H)-one analog”) is useful for treating or preventing a condition.

A composition comprising an effective amount of a tetracyclic 1H-indeno[1,2-b]pyridine-2(5H)-one analog and a physiologically acceptable carrier or vehicle is useful for treating or preventing a condition.

Also provided herein is a method of treating a disease associated with exposure to a reactive species, comprising administering to a subject in need thereof an effective amount of tetracyclic 1H-indeno[1,2-b]pyridine-2(5H)-one analog of Formula (I). Also provided herein is the use of one or more compounds of Formula (I) or a pharmaceutically acceptable salt thereof defined above in an effective amount in the manufacture of a medicament for administering to a subject in need of treatment or prevention of a disease associated with exposure to a reactive species. In one embodiment, the disease associated with exposure to a reactive species is selected from the group consisting of an inflammatory disease, a reperfusion injury, diabetes mellitus, a diabetic complication, reoxygenation injury resulting from organ transplantation, an ischemic condition, a neurodegenerative disease, renal failure, a vascular disease, a cardiovascular disease, cancer, an ocular or opthalmologic disease, a complication of prematurity, cardiomyopathy, retinopathy, nephropathy, contrast induced nephropathy, neuropathy, erectile dysfunction or urinary incontinence.

Other features and advantages of the invention will become apparent from the following detailed description, and from the claims.

### BRIEF DESCRIPTION OF THE DRAWINGS

FIG. 1: Shows graphically, the effect of a PARP inhibitor (Example 17) on plasma creatinine levels in diabetic rats exhibiting contrast-induced nephropathy.

FIG. 2: Shows graphically, the effect of a PARP inhibitor (Example 17) on plasma NGAL levels in diabetic rats exhibiting contrast-induced nephropathy.

FIG. 3: Shows graphically, the effect of a PARP inhibitor (Example 17) on urine NGAL levels in diabetic rats exhibiting contrast-induced nephropathy.

FIG. 4: Shows graphically, the effect of a PARP inhibitor (Example 17) on kidney histological scoring in diabetic rats exhibiting contrast-induced nephropathy.

FIG. 5: Shows graphically, the effect of a PARP inhibitor (Example 17) on urine αGST levels in diabetic rats exhibiting contrast-induced nephropathy.

FIG. 6: Shows graphically the manner in which the contrast agent and drugs were administered to diabetic rats exhibiting contrast-induced nephropathy in Example VII.

All patents and publications cited in this specification are incorporated by reference in their entirety.

### 4. DETAILED DESCRIPTION OF THE INVENTION

### 4.1 Definitions and Abbreviations

The following definitions are used in connection with the Compounds of the present invention;

The term “halo” as used herein refers to —F, —Cl, —Br or —I.

The term “—C₁-C₆ alkyl” as used herein, refers to a straight chain or branched non-cyclic hydrocarbon having from 1 to 6 carbon atoms, wherein one of the hydrocarbon's hydrogen atoms has been replaced by a single bond. Representative straight chain —C₁-C₆ alkyls include -methyl, -ethyl, -n-propyl, -n-butyl, -n-pentyl and -n-hexyl. Representative branched —C₁-C₆ alkyls include -isopropyl, -sec-butyl, -isobutyl, -tent-butyl, -isopentyl, -neopentyl, -1-methylbutyl, -isohexyl, -neohexyl, -2-methylbutyl, -3-methylbutyl, -1,1-dimethylpropyl and -1,2-dimethylpropyl.

A “C₁-C₆ alkylene” refers to a C₁-C₆ alkyl group, as defined above, wherein two of the C₁-C₆ alkyl group's hydrogen atoms have been replaced with a bond.

The term “—C₁-C₁₀ alkyl” as used herein, refers to a straight chain or branched non-cyclic hydrocarbon having from 1 to 10 carbon atoms, wherein one of the hydrocarbon's hydrogen atoms has been replaced by a single bond. Representative —C₁-C₁₀ alkyls include, methyl, ethyl, propyl, butyl, pentyl, hexyl, heptyl, octyl, -nonyl, decyl, isopropyl, isobutyl, sec-butyl and tert-butyl, isopentyl, neopentyl, isohexyl, isoheptyl, isooctyl, isononyl and isodecyl.

The term “—C₃-C₈ monocyclic cycloalkyl” as used herein, refers to a saturated cyclic hydrocarbon having from 3 to 8 carbon atoms. Representative —C₃-C₈ monocyclic cycloalkyls include -cyclopropyl, -cyclobutyl, -cyclopentyl, -cyclohexyl, -cycloheptyl and -cyclooctyl.

A “nitrogen containing 3- to 8-membered monocyclic heterocycle” refers to a monocyclic 3- to 8-membered aromatic or non-aromatic monocyclic cycloalkyl group having one or more double bonds and in which one of the cycloalkyl group's ring carbon atoms has been replaced with a nitrogen atom and 0-4 of the cycloalkyl group's remaining ring carbon atoms are independently replaced with a N, O or S atom or a S(O₂) group. The nitrogen containing 3- to 8-membered monocyclic heterocycles can be attached via a nitrogen, sulfur, or carbon atom. Representative examples of nitrogen-containing-3- to 8-membered monocyclic heterocycles include, but are not limited to, piperidinyl, piperazinyl, piperazinonenyl, pyrrolyl, piperidonyl, oxazinyl, thiazinyl, diazinyl, triazinyl, tetrazinyl, imidazolyl, tetrazolyl, pyrrolidinyl, isoxazolyl, pyridinyl, oxazolyl, thiazolyl, pyrazolyl, triazolyl, pyrimidinyl, morpholinyl, furuzanyl, pyrrolinyl, imidazolinyl, imidazolidinyl, pyrazolinyl, isothiazolyl, oxadiazolyl, thiadiazolyl, pyridazinyl, pyrazinyl, pyrazolidinyl, and thiomorpholinyl.

In one embodiment, the nitrogen-containing 3- to 8-membered monocyclic heterocycle is fully saturated or partially saturated.

A “3- to 8-membered monocyclic heterocycle” refers to a monocyclic 3- to 8-membered aromatic or non-aromatic monocyclic cycloalkyl in which 1-4 of the ring carbon atoms have been independently replaced with a N, O or S atom. The 3- to 8-membered monocyclic heterocycles can be attached via a nitrogen, sulfur, or carbon atom. Representative examples of a 3- to 8-membered monocyclic heterocycle group include, but are not limited to, nitrogen-containing 3- to 8-membered monocyclic heterocycles discussed above, tetrahydrofuranyl, dihydrofuranyl, pyranyl, dihydropyranyl, tetrahydropyranyl, thiopyranyl, dihydrothiopyranyl, tetrahydrothiopyranyl, dioxanyl, dithianyl, trithianyl, dioxolanyl, furanyl, and thiophenyl. In one embodiment, the 3- to 8-membered monocyclic heterocycle is a nitrogen-containing 3- to 8-membered monocyclic heterocycle. In another embodiment, the 3- to 8-membered monocyclic heterocycle is saturated or partially saturated.

A “subject” is a mammal, e.g., a human, mouse, rat, guinea pig, dog, cat, horse, cow, pig, or non-human primate, such as a monkey, chimpanzee, baboon or rhesus. In one embodiment, the subject is a human.

The phrase “pharmaceutically acceptable salt,” as used herein, is a salt of an acid and a basic nitrogen atom of a tetracyclic 1H-indeno[1,2-b]pyridine-2(5H)-one analog. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate (mesylate), ethanesulfonate, benzenesulfonate, p-toluenesulfonate, camphorsulfonate, and pamoate (i.e., 1,1′-methylene-bis-(2-hydroxy-3-naphthoate)) salts. The term “pharmaceutically acceptable salt” also refers to a salt of a tetracyclic 1H-indeno[1,2-b]pyridine-2(5H)-one analog having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” also includes a hydrate of a compound of the invention.

In one embodiment, the pharmaceutically acceptable salt is a mesylate salt.

An “effective amount” when used in connection with a tetracyclic 1H-indeno[1,2-b]pyridine-2(5H)-one analog is an amount that is effective for treating or preventing a Condition.

An “effective amount” when used in connection with another anticancer agent is an amount that is effective for treating or preventing cancer alone or in combination with a tetracyclic 1H-indeno[1,2-b]pyridine-2(5H)-one analog. “In combination with” includes administration within the same composition and via separate compositions. In the latter instance, the other anticancer agent is administered during a time when the tetracyclic 1H-indeno[1,2-b]pyridine-2(5H)-one analog exerts its prophylactic or therapeutic effect, or vice versa.

The term “reactive species” as used herein refers to a species that can injure a cell or tissue. Exemplary reactive species include oxidants, toxins and free radicals. Further exemplary reactive species include a reactive oxygen species, such as superoxide or peroxide, and a reactive nitrogen species, such as ⁻ONOO, nitric oxide, NO⁻, NOH, or ONO.

The term “contrast-induced nephropathy” (CIN) is to be understood as existing upon a 25% relative increase in serum creatinine levels within 24-72 hours of contrast agent administration in a given subject in the absence of other attributing factors.

The following abbreviations are used herein and have the indicated definitions: DMF is N,N-dimethylformamide, THF is tetrahydrofuran, DMAC is dimethylacetamide. DMSO is dimethylsulfoxide, Et is ethyl, Pr is n-propyl, i-Pr is isopropyl, EtOAc is ethyl acetate, EtOH is ethanol, Me is methyl, MS is mass spectrometry, Ts is tosylate, Tf is triflate, Ph is phenyl, NMR is Nuclear Magnetic Resonance, Ms is mesylate, LAH is lithium aluminum hydride and tBoc is t-butyloxycarbonyl.

### 4.1 Compounds of the Invention

In one aspect the invention provides a compound of Formula (I)

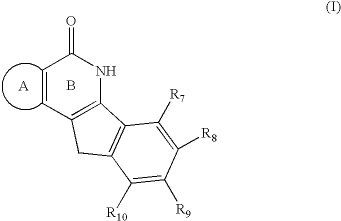

and pharmaceutically acceptable salts thereof,

wherein

A is a 4-7 membered monocyclic heterocycle ring or a —C₃-C₈ monocyclic ring,

each R₇, R₈, R₉ and R₁₀ is independently —H, -halo, —OH, —CN, —C₁-C₆ alkyl, —C₃-C₈ monocyclic cycloalkyl, —N(R^{a})₂, —C(O)O—C₁-C₆ alkyl, —(C₁-C₆ alkylene)-N(R^{a})₂, —C₁-C₆ alkyl, —N(Z₃)(Z₄), a nitrogen-containing 3- to 8-membered monocyclic heterocycle, —(C₁-C₆ alkylene)-N(Z₃)(Z₄), —(C₁-C₅ alkylene)-(a nitrogen-containing 3- to 8-membered monocyclic heterocycle), S(O)₂—C₁-C₆ alkyl or S(O)₂NH—C₁-C₆ alkyl; each of which other than —H, -halo, and —CN, is independently unsubstituted or substituted with one or more of -halo, —OH, —N(R^{a})₂, —CF₃, —C₁-C₆ alkyl, O—C₁-C₆ alkyl, —C₄-C₆ aryl, —C₄-C₆ aryl substituted with one or more halo, —C₁-C₆ alkylene-C₄-C₆ aryl, —C₁-C₆ alkylene-O—C₁-C₆ alkyl, —C₁-C₆ alkylene-(O—C₁-C₆ alkyl)₂, —C₁-C₆ alkylene-OH, —C₁-C₆ alkylene-N(R^{a})₂, —C₁-C₆ alkylene-C(O)—O—C₁-C₆ alkyl, —C(O)—O—C₁-C₆ alkyl, —C(O)—C₁-C₆ alkylene-OH, —C(O)—N(R^{a})₂, —C(O)—C₁-C₆ alkylene-N(R^{a})₂, a 3- to 7-membered monocyclic heterocycle, or —N(Z₃)(Z₄),

wherein each occurrence of R^{a} is independently —H, halo, benzyl, alkyl, —C₁-C₁₀ alkyl-O—C₁-C₁₀ alkyl, C₁-C₁₀ alkyl-OH, —C₁-C₆ alkyl-3-8 membered monocyclic heterocycle, —C₃-C₈ monocyclic alkyl, —C₃-C₈ monocyclic alkyl substituted with one or more of —OH, —C₁-C₆ alkyl, —C₁-C₆ alkylene-NH₂, —O—C₁-C₆ alkylene or —C₁-C₆ alkylene-OH; a 3- to 8-membered monocyclic heterocycle, a 3- to 8-membered monocyclic heterocycle substituted with one or more of —OH, —C₁-C₆ alkyl or —C₁-C₆ alkylene-OH, —(C₁-C₆ alkylene)-O—C₁-C₆ alkyl, C(O)—(C₁-C₆ alkylene)-N—(C₁-C₆ alkyl)₂, —(C₁-C₆ alkylene)-(a 3- to 8-membered monocyclic heterocycle), —C(O)—C₁-C₆ alkylene-S(O)—C₁-C₆ alkyl, —C(O)—C₁-C₆ alkylene-S—C₁-C₆ alkyl, —C(O)—C₁-C₆ alkyl optionally substituted with NH₂ or OH;

wherein N, Z₃ and Z₄ are taken together to form a 3- to 8-membered monocyclic heterocycle which is unsubstituted or substituted with one or more of halo, —OH, —CF₃, —C₁-C₆ alkyl, —NH₂, —NH(C₁-C₆ alkyl), —N(C₁-C₆ alkyl)₂, —C₁-C₆ alkylene-OH, —C₁-C₆ alkylene-halo, —C₁-C₆ alkylene-NH₂, —C₃-C₈ monocyclic alkyl, a 3- to 8-membered monocyclic heterocycle, —C₁-C₆ alkylene-C₃-C₈-monocyclic alkyl, —C(O)C₃-C₈ monocyclic alkyl, —C(±)-3-8 membered monocyclic heterocycle, —C(O)—NH₂, —C(O)—(C₁-C₆ alkylene)-NH—C₁-C₆ alkyl; C(O)—(C₁-C₆ alkylene)-N—(C₁-C₆ alkyl)₂; C(O)—C₁-C₆ alkyl optionally substituted with NH₂ or OH, —C₁-C₆ alkylene-O—C₁-C₆ alkyl or —O—C₁-C₆ alkyl.

In another aspect, provided herein is a compound of Formula (I) as defined above wherein A is selected from

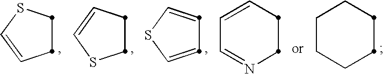

where represents the points of attachment to the B ring.
In one aspect A is

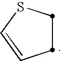

In another aspect A is

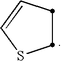

In a further aspect A is

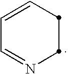

In another aspect A is

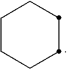

Also provided herein is a compound of Formula (I) as defined above wherein R₇ to R₁₀ is independently selected from the following:

R₇, R₈, R₉ or R₁₀

| | |
|---|---|
| 1 | —H |
| 2 | —Cl, —Br, or —F |
| 3 | —Me, or —CF₃ |
| 4 | —OH, OCH₃, or OCF₃ |
| 5 | —NH₂ or —NHMe |
| 6 | 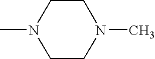 |
| 7 | 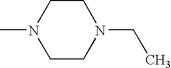 |
| 8 | 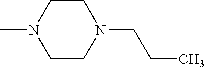 |
| 9 | 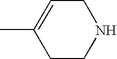 |
| 10 | 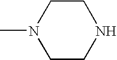 |
| 11 | 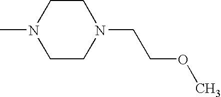 |
| 12 | 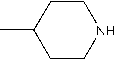 |
| 13 | 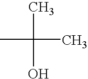 |
| 14 | 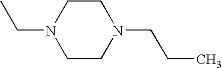 |
| 15 | 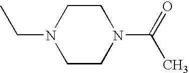 |
| 16 | 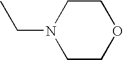 |
| 17 | 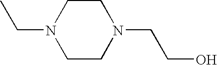 |
| 18 | 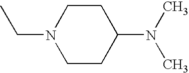 |
| 19 | 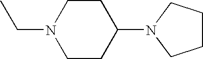 |
| 20 | 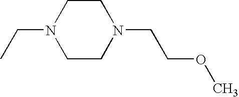 |
| 21 | 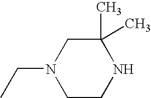 |
| 22 | 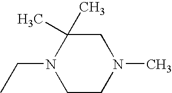 |
| 23 | 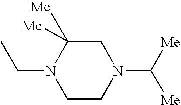 |
| 24 | 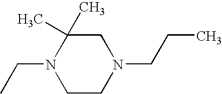 |
| 25 | 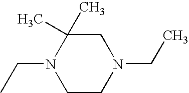 |
| 26 | 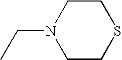 |
| 27 | 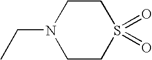 |
| 28 | 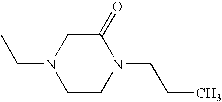 |
| 29 | 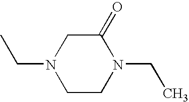 |
| 30 | 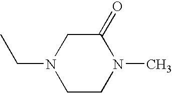 |
| 31 | 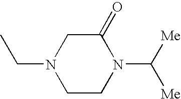 |
| 32 | 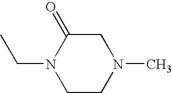 |
| 33 | 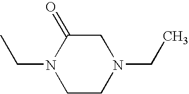 |
| 34 | 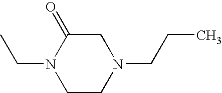 |
| 35 | 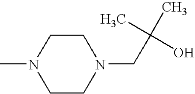 |
| 36 | 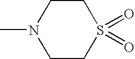 |
| 37 | 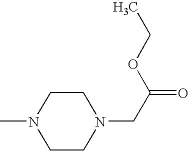 |
| 38 | 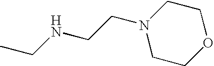 |
| 39 | 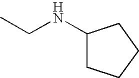 |
| 40 | 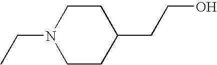 |
| 41 | 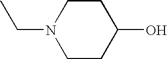 |
| 42 | 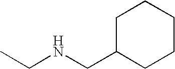 |
| 43 | 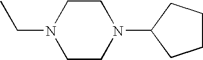 |
| 44 | 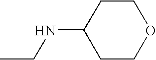 |
| 45 | 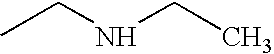 |
| 46 | 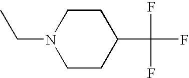 |
| 47 | 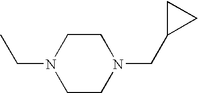 |
| 48 | 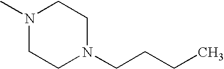 |
| 49 | 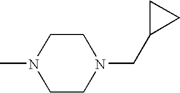 |
| 50 | 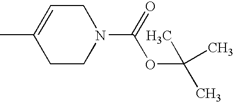 |
| 51 | 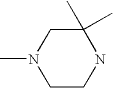 |
| 52 | 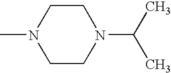 |
| 53 | 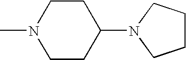 |
| 54 | 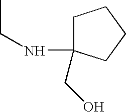 |
| 55 | 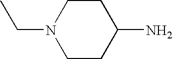 |
| 56 | 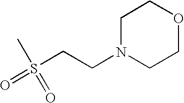 |
| 57 | 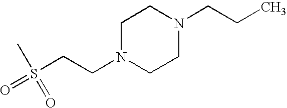 |
| 58 | 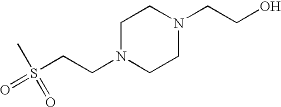 |
| 59 | 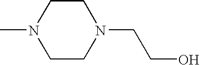 |
| 60 | 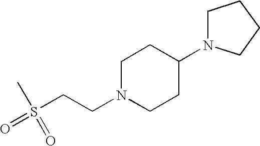 |
| 61 | 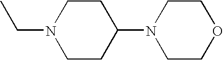 |
| 62 | 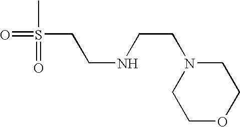 |
| 63 | 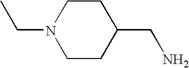 |
| 64 | 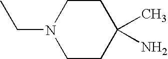 |
| 65 | 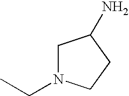 |
| 66 | 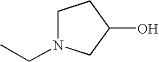 |
| 66 | 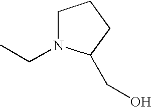 |
| 67 | 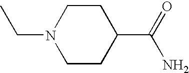 |
| 68 | 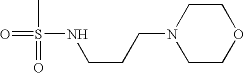 |
| 69 | 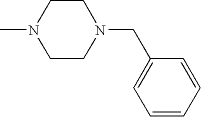 |
| 70 | 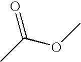 |
| 71 | 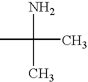 |
| 72 | 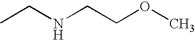 |
| 73 | 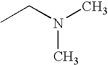 |
| 74 | 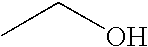 |
| 75 | 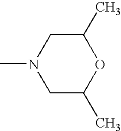 |
| 76 | 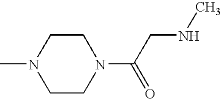 |
| 77 | 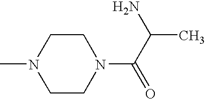 |
| 78 | 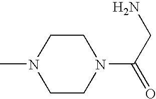 |
| 79 | 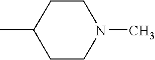 |
| 80 | 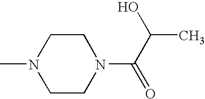 |
| 81 | 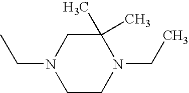 |
| 82 | 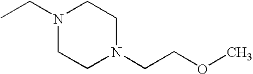 |
| 83 | 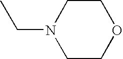 |
| 84 | 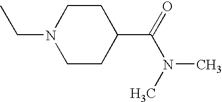 |
| 85 | 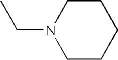 |
| 86 | 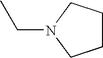 |
| 87 | 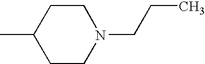 |
| 88 | 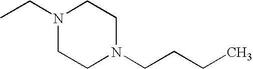 |
| 89 | 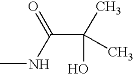 |
| 90 | 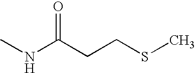 |
| 91 | 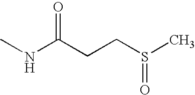 |
| 92 | 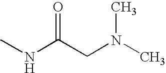 |
| 93 | 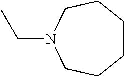 |
| 94 | 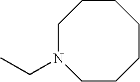 |
| 95 | 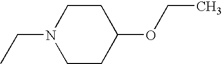 |
| 96 | 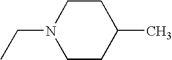 |
| 97 | 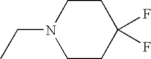 |
| 98 | 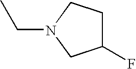 |
| 99 | 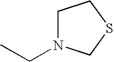 |
| 100 | 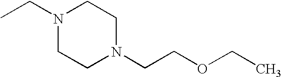 |
| 101 | 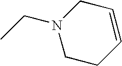 |
| 101 | 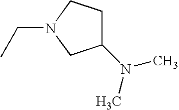 |
| 102 | 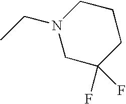 |
| 103 | 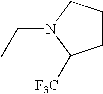 |
| 104 | 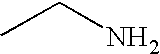 |
| 105 | 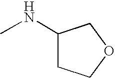 |
| 106 | 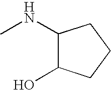 |
| 107 | 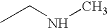 |
| 108 | 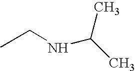 |
| 109 | 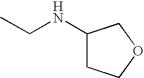 |
| 110 | 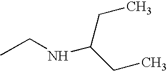 |
| 111 | 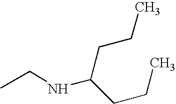 |
| 112 | 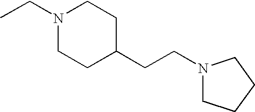 |
| 113 | 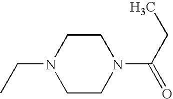 |
| 114 | 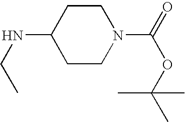 |
| 115 | 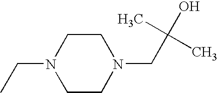 |
| 116 | 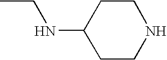 |
| 117 | 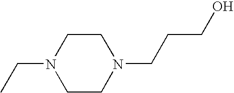 |
| 118 | 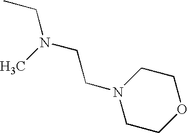 |
| 119 | 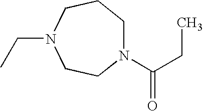 |
| 120 | 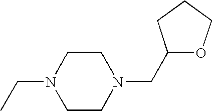 |
| 121 | 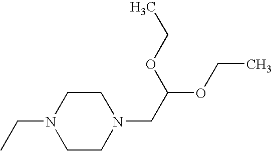 |
| 122 | 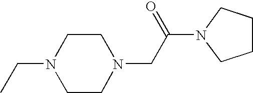 |
| 123 | 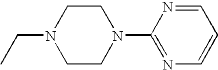 |
| 124 | 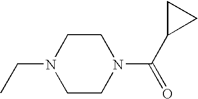 |
| 125 | 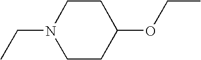 |
| 126 | —CN |
| 127 | 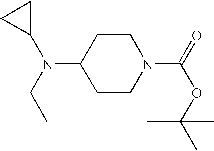 |
| 128 | 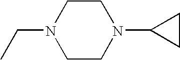 |
| 129 | 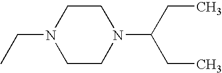 |
| 130 | 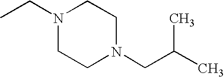 |
| 131 | 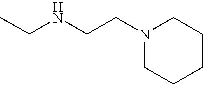 |
| 132 | 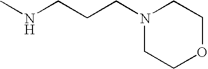 |
| 133 | 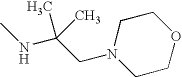 |
| 134 | 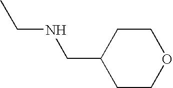 |
| 135 | 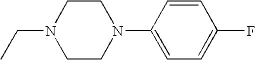 |
| 136 | 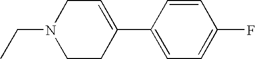 |
| 137 | 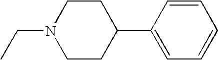 |
| 138 | 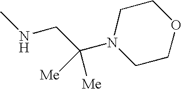 |
| 139 | 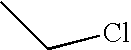 |
| 140 | 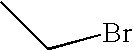 |
| 141 | 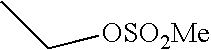 |
| 142 | 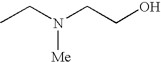 |
| 143 | 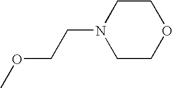 |
| 144 | 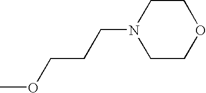 |
| 145 | 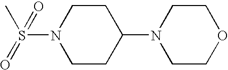 |
| 146 | 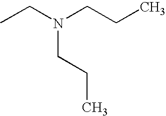 |
| 147 | 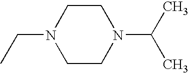 |
| 148 | 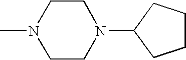 |
| 149 | 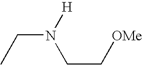 |
| 150 | 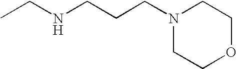 |
| 151 | 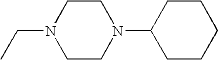 |
| 152 | 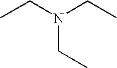 |
| 153 | 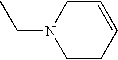 |
| 154 | 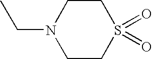 |

In another embodiment there is provided a compound of Formula (I) or a pharmaceutically acceptable salt thereof wherein R₉ or R₁₀ is selected from

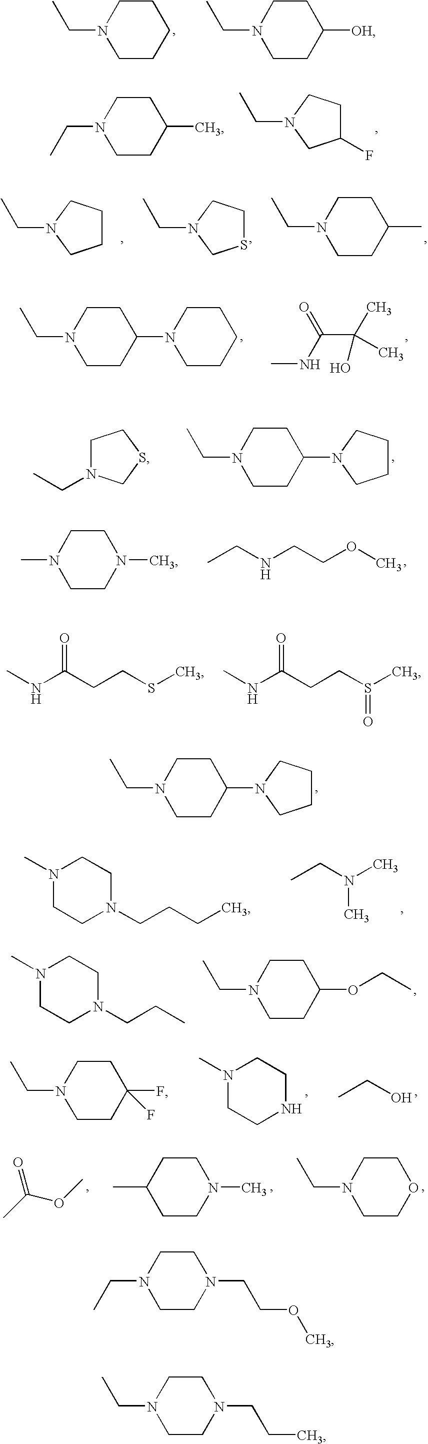

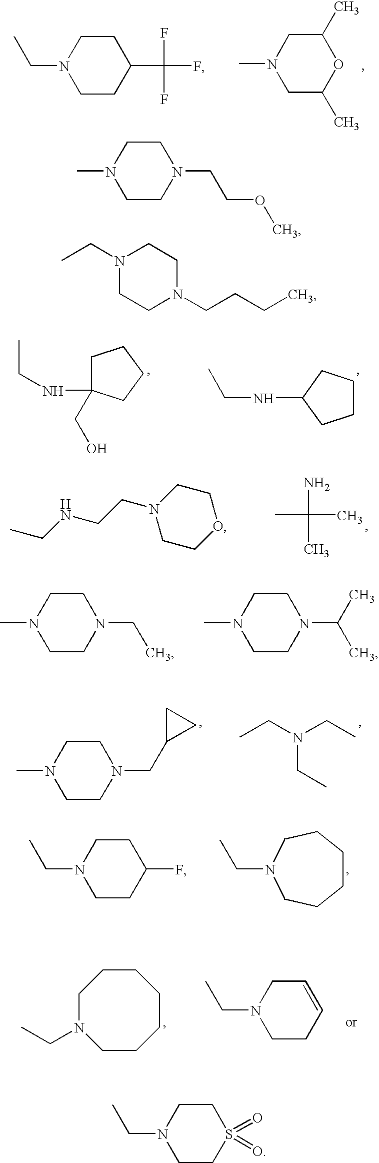

In another aspect, provided herein is a compound of Formula (I) or a pharmaceutically acceptable salt thereof, wherein A is;

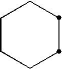

each R₇, R₈, and R₉ are H; and

R₁₀ is —(C₁-C₃ alkylene)-N(Z₃)(Z₄);

wherein N, Z₃ and Z₄ are taken together to form piperidine, azepane, or pyrrolidine, wherein the piperidine, azepane, or pyrrolidine rings are independently substituted with halo or —C₁-C₃ alkyl.

In another aspect, there is provided a compound of Formula I or a pharmaceutically acceptable salt thereof selected from:
10-((4-hydroxypiperidin-1-yl)methyl)-3,4,6,11-tetrahydro-1H-indeno[1,2-c]isoquinolin-5(2H)-one,
10-(piperidin-1-ylmethyl)-3,4,6,11-tetrahydro-1H-indeno[1,2-c]isoquinolin-5(2H)-one,
10-(azepan-1-ylmethyl)-3,4,6,11-tetrahydro-1H-indeno[1,2-c]isoquinolin-5(2H)-one,
10-(pyrrolidin-1-ylmethyl)-3,4,6,11-tetrahydro-1H-indeno[1,2-c]isoquinolin-5(2H)-one,
10-((4-ethoxypiperidin-1-yl)methyl)-3,4,6,11-tetrahydro-1H-indeno[1,2-c]isoquinolin-5(2H)-one,
10-(azocan-1-ylmethyl)-3,4,6,11-tetrahydro-1H-indeno[1,2-c]isoquinolin-5(2H)-one,
10-((4,4-difluoropiperidin-1-yl)methyl)-3,4,6,11-tetrahydro-1H-indeno[1,2-c]isoquinolin-5(2H)-one,
10-(thiazolidin-3-ylmethyl)-3,4,6,11-tetrahydro-1H-indeno[1,2-c]isoquinolin-5(2H)-one,
(S)-10-((3-fluoropyrrolidin-1-yl)methyl)-3,4,6,11-tetrahydro-1H-indeno[1,2-c]isoquinolin-5(2H)-one,
(R)-10-((3-fluoropyrrolidin-1-yl)methyl)-3,4,6,11-tetrahydro-1H-indeno[1,2-c]isoquinolin-5(2H)-one,
10-((4-methylpiperidin-1-yl)methyl)-3,4,6,11-tetrahydro-1H-indeno[1,2-c]isoquinolin-5(2H)-one,
10-(1,4′-bipiperidin-1′-ylmethyl)-3,4,6,11-tetrahydro-1H-indeno[1,2-c]isoquinolin-5(2H)-one,
10-((4-(pyrrolidin-1-yl)piperidin-1-yl)methyl)-3,4,6,11-tetrahydro-1H-indeno[1,2-c]isoquinolin-5(2H)-one,
10-((cyclopentylamino)methyl)-3,4,6,11-tetrahydro-1H-indeno[1,2-c]isoquinolin-5(2H)-one,
10-((2-methoxyethylamino)methyl)-3,4,6,11-tetrahydro-1H-indeno[1,2-c]isoquinolin-5(2H)-one,
10-((dimethylamino)methyl)-3,4,6,11-tetrahydro-1H-indeno[1,2-c]isoquinolin-5(2H)-one), 10-(hydroxymethyl)-3,4,6,11-tetrahydro-1H-indeno[1,2-c]isoquinolin-5(2H)-one, methyl 5-oxo-2,3,4,5,6,11-hexahydro-1H-indeno[1,2-c]isoquinoline-9-carboxylate,
10-(piperazin-1-ylmethyl)-3,4,6,11-tetrahydro-1H-indeno[1,2-c]isoquinolin-5(2H)-one,
10-(2,6-dimethylmorpholino)-3,4,6,11-tetrahydro-1H-indeno[1,2-c]isoquinolin-5(2H)-one,
9-(hydroxymethyl)-3,4,6,11-tetrahydro-1H-indeno[1,2-c]isoquinolin-5(2H)-one,
9-((4-propylpiperazin-1-yl)methyl)-3,4,6,11-tetrahydro-1H-indeno[1,2-c]isoquinolin-5(2H)-one,
9-((4-(2-methoxyethyl)piperazin-1-yl)methyl)-3,4,6,11-tetrahydro-1H-indeno[1,2-c]isoquinolin-5(2H)-one,
9-((2-methoxyethylamino)methyl)-3,4,6,11-tetrahydro-1H-indeno[1,2-c]isoquinolin-5(2H)-one,
9-(morpholinomethyl)-3,4,6,11-tetrahydro-1H-indeno[1,2-c]isoquinolin-5(2H)-one,
9-((4-hydroxypiperidin-1-yl)methyl)-3,4,6,11-tetrahydro-1H-indeno[1,2-c]isoquinolin-5(2H)-one,
9-(pyrrolidin-1-ylmethyl)-3,4,6,11-tetrahydro-1H-indeno[1,2-c]isoquinolin-5(2H)-one,
9-((cyclopentylamino)methyl)-3,4,6,11-tetrahydro-1H-indeno[1,2-c]isoquinolin-5(2H)-one,
10-(4-propylpiperazin-1-yl)-3,4,6,11-tetrahydro-1H-indeno[1,2-c]isoquinolin-5(2H)-one,
9-(4-butylpiperazin-1-yl)-3,4,6,11-tetrahydro-1H-indeno[1,2-c]isoquinolin-5(2H)-one,
10-(4-(2-methoxyethyl)piperazin-1-yl)-3,4,6,11-tetrahydro-1H-indeno[1,2-c]isoquinolin-5(2H)-one,
9-((4-butylpiperazin-1-yl)methyl)-3,4,6,11-tetrahydro-1H-indeno[1,2-c]isoquinolin-5(2H)-one,
9-((2-morpholinoethylamino)methyl)-3,4,6,11-tetrahydro-1H-indeno[1,2-c]isoquinolin-5(2H)-one,
10-((1-(hydroxymethyl)cyclopentylamino)methyl)-3,4,6,11-tetrahydro-1H-indeno[1,2-c]isoquinolin-5(2H)-one,
2-hydroxy-2-methyl-N-(5-oxo-2,3,4,5,6,11-hexahydro-1H-indeno[1,2-c]isoquinolin-10-yl)propanamide,
3-(methylthio)-N-(5-oxo-2,3,4,5,6,11-hexahydro-1H-indeno[1,2-c]isoquinolin-10-yl)propanamide,
3-(methylsulfinyl)-N-(5-oxo-2,3,4,5,6,11-hexahydro-1H-indeno[1,2-c]isoquinolin-10-yl)propanamide,
methyl 5-oxo-2,3,4,5,6,11-hexahydro-1H-indeno[1,2-c]isoquinoline-10-carboxylate,
10-(2-aminopropan-2-yl)-3,4,6,11-tetrahydro-1H-indeno[1,2-c]isoquinolin-5(2H)-one,
9-((diethylamino)methyl)-3,4,6,11-tetrahydro-1H-indeno[1,2-c]isoquinolin-5(2H)-one,
9-((4,4-difluoropiperidin-1-yl)methyl)-3,4,6,11-tetrahydro-1H-indeno[1,2-c]isoquinolin-5(2H)-one,
9-((4-fluoropiperidin-1-yl)methyl)-3,4,6,11-tetrahydro-1H-indeno[1,2-c]isoquinolin-5(2H)-one,
10-((diethylamino)methyl)-3,4,6,11-tetrahydro-1H-indeno[1,2-c]isoquinolin-5(2H)-one,
10-((4-fluoropiperidin-1-yl)methyl)-3,4,6,11-tetrahydro-1H-indeno[1,2-c]isoquinolin-5(2H)-one,
10-((5,6-dihydropyridin-1(2H)-yl)methyl)-3,4,6,11-tetrahydro-1H-indeno[1,2-c]isoquinolin-5(2H)-one and
10-(thiomorpholinomethyl-1,1-dioxide)-3,4,6,11-tetrahydro-1H-indeno[1,2-c]isoquinolin-5(2H)-one.

In another aspect, there is provided a compound of Formula (I) or a pharmaceutically acceptable salt thereof selected from the following:
9-((4-(trifluoromethyl)piperidin-1-yl)methyl)-5,10-dihydro-4H-indeno[1,2-b]thieno[3,2-d]pyridin-4-one,
9-((cyclopentylamino)methyl)-5,10-dihydro-4H-indeno[1,2-b]thieno[3,2-d]pyridin-4-one,
9-((4-(trifluoromethyl)piperidin-1-yl)methyl)-5,10-dihydro-4H-indeno[1,2-b]thieno[2,3-d]pyridin-4-one, and
9-((cyclopentylamino)methyl)-5,10-dihydro-4H-indeno[1,2-b]thieno[2,3-d]pyridin-4-one.

In another aspect there is provided a compound of Formula (I) or a pharmaceutically acceptable salt thereof selected from the following:
10-(4-(2-methoxyethyl)piperazin-1-yl)-6,11-dihydro-5H-indeno[1,2-h][1,6]naphthyridin-5-one,
10-(4-methylpiperazin-1-yl)-6,11-dihydro-5H-indeno[1,2-h][1,6]naphthyridin-5-one,
10-(hydroxymethyl)-6,11-dihydro-5H-indeno[1,2-h][1,6]naphthyridin-5-one,
10-(4-butylpiperazin-1-yl)-6,11-dihydro-5H-indeno[1,2-h][1,6]naphthyridin-5-one,
10-(4-ethylpiperazin-1-yl)-6,11-dihydro-5H-indeno[1,2-h][1,6]naphthyridin-5-one,
10-(4-isopropylpiperazin-1-yl)-6,11-dihydro-5H-indeno[1,2-h][1,6]naphthyridin-5-one,
10-(4-(cyclopropylmethyl)piperazin-1-yl)-6,11-dihydro-5H-indeno[1,2-h][1,6]naphthyridin-5-one, and
10-((cyclopentylamino)methyl)-6,11-dihydro-5H-indeno[1,2-h][1,6]naphthyridin-5-one.

In another aspect there is provided a compound of Formula (I) being 10-(azepan-1-ylmethyl)-3,4,6,11-tetrahydro-1H-indeno[1,2-c]isoquinolin-5(2H)-one or a pharmaceutically acceptable salt thereof.

In another aspect there is provided a compound of Formula (I) being 10-(piperidin-1-ylmethyl)-3,4,6,11-tetrahydro-1H-indeno[1,2-c]isoquinolin-5(2H)-one or a pharmaceutically acceptable salt thereof.

In another aspect there is provided a compound of Formula (I) being 10-((4-methylpiperidin-1-yl)methyl)-3,4,6,11-tetrahydro-1H-indeno[1,2-c]isoquinolin-5(2H)-one or a pharmaceutically acceptable salt thereof.

In another aspect there is provided a compound of Formula (I) being (S)-10-((3-fluoropyrrolidin-1-yl)methyl)-3,4,6,11-tetrahydro-1H-indeno[1,2-c]isoquinolin-5(2H)-one or a pharmaceutically acceptable salt thereof.

The invention provides compositions comprising an effective amount of a compound of Formula (I), or a pharmaceutically acceptable salt thereof and a physiologically acceptable carrier or vehicle.

### 4.3 Treatment or Prevention of a Condition

In accordance with the invention, a tetracyclic 1H-indeno[1,2-b]pyridine-2(5H)-one analog is useful for treatment or prevention of a Condition as set forth below.

### 4.2.1 Treatment or Prevention of an Inflammatory Disease

The tetracyclic 1H-indeno[1,2-b]pyridine-2(5H)-one analogs provided herein are useful for treating or preventing an inflammatory disease. An inflammatory disease can arise where there is an inflammation of the body tissue. These include local inflammatory responses and systemic inflammation. Examples of inflammatory diseases include, but are not limited to, lupus; pancreatitis; macular degeneration; chronic obstructive pulmonary disease; organ transplant rejection; a chronic inflammatory disease of a joint, including arthritis, rheumatoid arthritis, osteoarthritis and a bone disease associated with increased bone resorption; an inflammatory bowel disease such as ileitis, ulcerative colitis, Barrett's syndrome, and Crohn's disease; an inflammatory lung disease such as asthma, adult respiratory distress syndrome, and chronic obstructive airway disease; an inflammatory disease of the eye a chronic inflammatory disease of the gum, including gingivitis and periodontitis; tuberculosis; leprosy; an inflammatory disease of the kidney including a uremic complication, glomerulonephritis and nephrosis; an inflammatory disease of the skin including sclerodermatitis, psoriasis and eczema; an inflammatory disease of the central nervous system, including a chronic demyelinating disease of the nervous system, multiple sclerosis, AIDS-related neurodegeneration and Alzheimer's disease, infectious meningitis, encephalomyelitis, Huntington's disease, amyotrophic lateral sclerosis and viral or autoimmune encephalitis; as well as any other disease that can have a significant inflammatory component, including preeclampsia, chronic liver failure, and brain and spinal cord trauma. The inflammatory disease can also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer.

In one embodiment, the inflammatory disease is an inflammatory disease of a joint, a chronic inflammatory disease of the gum, an inflammatory bowel disease, an inflammatory lung disease, an inflammatory disease of the central nervous system, an inflammatory disease of the eye, gram-positive shock, gram negative shock, Rickettsial shock, fungal shock, hemorrhagic shock, anaphylactic shock, traumatic shock or chemotherapeutic shock.

In another embodiment, the inflammatory disease is an ocular inflammatory disease, including corneal dystrophy, trachoma, onchocerciasis, uveitis, sympathetic ophthalmitis and endophthalmitis;uveitis, retinitis, cytomegalovirus retinitis, also known as CMV retinitis, blepharitis, conjunctivitis, scleritistemporal arteritis, scleritis, conjunctivitis, keratitis, iritis, iridocyclitis, and progressive outer retinal necrosis (PORN), also known as Varicella zoster virus retinitis (VZVR) or a chronic and sub-clinical inflammatory disease such as glaucoma, aged macular degeneration or diabetic retinopathy.

### 4.2.2 Treatment or Prevention of a Reperfusion injury

The tetracyclic 1H-indeno[1,2-b]pyridine-2(5H)-one analogs are useful for treating or preventing a reperfusion injury. Reperfusion refers to the process whereby blood-flow in the blood vessels is resumed following ischemia, such as occurs following constriction or obstruction of the vessel. Reperfusion injury can result following a naturally occurring episode, such as a myocardial infarction, stroke, or during a surgical procedure where blood flow in vessels is intentionally or unintentionally blocked. Examples of reperfusion injuries include, but are not limited to, intestinal reperfusion injury, myocardial reperfusion injury, and reperfusion injury resulting from cardiopulmonary bypass surgery, aortic aneurysm repair surgery, carotid endarterectomy surgery, and hemorrhagic shock.

In one embodiment, the reperfusion injury results from cardiopulmonary bypass surgery, aortic aneurysm repair surgery, carotid endarterectomy surgery or hemorrhagic shock.

In another embodiment, the reperfusion injury is stroke or myocardial infarction.

### 4.2.3 Treatment or Prevention of Reoxygenation Injury Resulting from Organ Transplantation

The tetracyclic 1H-indeno[1,2-b]pyridine-2(5H)-one analogs are useful for treating or preventing reoxygenation injury resulting from organ transplantation. Examples of reoxygenation injuries include, but are not limited to, transplantation of one or more of the following: heart, lung, liver, kidney, pancreas, intestine and cornea.

In one embodiment, reoxygenation injury resulting from organ transplantation occurs during the organ transplantation.

The tetracyclic 1H-indeno[1,2-b]pyridine-2(5H)-one analogs are also useful for treating or preventing allograph rejection. Accordingly, the invention provides methods for treating or preventing allograph rejection, comprising administering an effective amount of a tetracyclic 1H-indeno[1,2-b]pyridine-2(5H)-one analog to a subject in need thereof. In one embodiment, the methods further comprise administering an effective amount of another agent useful for treating or preventing allograph rejection. The other agent includes, but is not limited to, SK-506 and cyclosporine.

### 4.2.4 Treatment or Prevention of an Ischemic Condition

The tetracyclic 1H-indeno[1,2-b]pyridine-2(5H)-one analogs are useful for treating or preventing an ischemic condition. Examples of ischemic conditions include, but are not limited to, stable angina, unstable angina, myocardial ischemia, hepatic ischemia, mesenteric artery ischemia, ischemic heart disease, intestinal ischemia, critical limb ischemia, chronic critical limb ischemia, cerebral ischemia, acute cardiac ischemia, and an ischemic disease of the central nervous system, such as stroke or cerebral ischemia.

In one embodiment, the ischemic condition is myocardial ischemia, stable angina, unstable angina, stroke, ischemic heart disease or cerebral ischemia.

### 4.2.5 Treatment or Prevention of Renal Failure

The tetracyclic 1H-indeno[1,2-b]pyridine-2(5H)-one analogs are useful for treating or preventing renal failure.

In one embodiment, the renal failure is chronic renal failure.

In another embodiment, the renal failure is acute renal failure.

### 4.2.6 Treatment or Prevention of a Vascular Disease

The tetracyclic 1H-indeno[1,2-b]pyridine-2(5H)-one analogs are useful for treating or preventing a vascular disease other than a cardiovascular disease. Examples of vascular diseases include, but are not limited to, hemorrahgic stroke, peripheral arterial occlusion, thromboangitis obliterans, Reynaud's disease and phenomenon, acrocyanosis, erythromelalgia, venous thrombosis, varicose veins, arteriovenous fistula, lymphedema, and lipedema.

In embodiment, the vascular disease is peripheral arterial occlusion, thromboangitis obliterans, Reynaud's disease and phenomenon, acrocyanosis, erythromelalgia, venous thrombosis, varicose veins, arteriovenous fistula, lymphedema or lipedema.

### 4.2.7 Treatment or Prevention of a Cardiovascular Disease

The tetracyclic 1H-indeno[1,2-b]pyridine-2(5H)-one analogs are useful for treating or preventing a cardiovascular disease. Examples of cardiovascular diseases include, but are not limited to, congestive heart failure (such as chronic or acute heart failure), atherosclerosis, hypercholesterolemia, circulatory shock, cardiomyopathy, cardiac transplant, myocardial infarction, and a cardiac arrhythmia, such as atrial fibrillation, supraventricular tachycardia, atrial flutter, and paroxysmal atrial tachycardia.

In one embodiment, the cardiovascular disease is chronic heart failure.

In another embodiment, the cardiovascular disease is acute heart failure.

In yet another embodiment, the cardiovascular disease is cardiac arrhythmia.

In still another embodiment, the cardiac arrhythmia is atrial fibrillation, supraventricular tachycardia, atrial flutter or paroxysmal atrial tachycardia.

In one embodiment, the cardiovascular disease is chronic heart failure, atrial fibrillation, supraventricular tachycardia, atrial flutter or paroxysmal atrial tachycardia.

### 4.2.8 Treatment or Prevention of Diabetes Mellitus or a Diabetic Complication

The tetracyclic 1H-indeno[1,2-b]pyridine-2(5H)-one analogs are useful for treating or preventing diabetes mellitus or one or more of its complications. Examples of diabetes mellitus include, but are not limited to, Type I diabetes (Insulin Dependent Diabetes Mellitus), Type II diabetes (Non-Insulin Dependent Diabetes Mellitus), gestational diabetes, autoimmune diabetes, insulinopathies, diabetes due to pancreatic disease, diabetes associated with other endocrine diseases (such as Cushing's Syndrome, acromegaly, pheochromocytoma, glucagonoma, primary aldosteronism or somatostatinoma), Type A insulin resistance syndrome, Type B insulin resistance syndrome, lipatrophic diabetes, and diabetes induced by B-cell toxins.

The tetracyclic 1H-indeno[1,2-b]pyridine-2(5H)-one analogs are useful for treating or preventing a complication of diabetes mellitus. Examples of complications of diabetes mellitus include, but are not limited to, diabetic cataract, glaucoma, retinopathy, nephropathy (such as microalbuminuria or progressive diabetic nephropathy), polyneuropathy, gangrene of the feet, immune-complex vasculitis, systemic lupus erythematosus (SLE), atherosclerotic coronary arterial disease, peripheral arterial disease, nonketotic hyperglycemic-hyperosmolar coma, mononeuropathies, autonomic neuropathy, foot ulcers, joint problems, and a skin or mucous membrane complication (such as an infection, a shin spot, a candidal infection or necrobiosis lipoidica diabeticorumobesity), hyperlipidemia, hypertension, syndrome of insulin resistance, coronary artery disease, retinopathy, neuropathy (such as diabetic neuropathy, polyneuropathy or mononeuropathy), autonomic neuropathy, a foot ulcer, a joint problem, a fungal infection, cardiomyopathy, and a bacterial infection.

In one embodiment diabetes mellitus is Type I diabetes mellitus or Type II diabetes mellitus.

### 4.2.9 Treatment or Prevention of a Neurodegenerative Disease

The tetracyclic 1H-indeno[1,2-b]pyridine-2(5H)-one analogs are useful for treating or preventing a neurodegenerative disease. Examples of neurodegenerative diseases include, but are not limited to, Alzheimer's disease, Parkinson's disease and amyotrophic lateral sclerosis.

### 4.2.10 Treatment or Prevention of Cancer

The tetracyclic 1H-indeno[1,2-b]pyridine-2(5H)-one analogs are useful for treating or preventing cancer. Accordingly, the invention provides methods for treating or preventing cancer, comprising administering an effective amount of a tetracyclic 1H-indeno[1,2-b]pyridine-2(5H)-one analog to a subject in need thereof. In one embodiment, the methods further comprise administering an effective amount of another anticancer agent.

Examples of cancers include, but are not limited to, the cancers disclosed below in Table 1 and metastases thereof

TABLE 1

Solid tumors, including but not limited to:
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophageal cancer
stomach cancer
oral cancer
nasal cancer
throat cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
epithelial carcinoma
skin cancer
melanoma
metastatic melanoma
neuroblastoma
retinoblastoma
blood-borne cancers, including but not limited to:
acute lymphoblastic leukemia (“ALL”)
acute lymphoblastic B-cell leukemia
acute lymphoblastic T-cell leukemia
acute myeloblastic leukemia (“AML”)
acute promyelocytic leukemia (“APL”)
acute monoblastic leukemia
acute erythroleukemic leukemia
acute megakaryoblastic leukemia
acute myelomonocytic leukemia
acute nonlymphocyctic leukemia
acute undifferentiated leukemia
chronic myelocytic leukemia (“CML”)
chronic lymphocytic leukemia (“CLL”)
hairy cell leukemia
multiple myeloma
acute and chronic leukemias:
lymphoblastic
myelogenous
lymphocytic
myelocytic leukemias
Lymphomas:
Hodgkin's disease
non-Hodgkin's Lymphoma
Multiple myeloma
Waldenström's macroglobulinemia
Heavy chain disease
Polycythemia vera
Central nervous system lymphomas
CNS and Brain cancers:
glioma
pilocytic astrocytoma
astrocytoma
anaplastic astrocytoma
glioblastoma multiforme
medulloblastoma
craniopharyngioma
ependymoma
pinealoma
hemangioblastoma
acoustic neuroma
oligodendroglioma
meningioma
vestibular schwannoma
adenoma
metastatic brain tumor
meningioma
spinal tumor
medulloblastoma

In one embodiment, the cancer is lung cancer, breast cancer, colorectal cancer, prostate cancer, a leukemia, a lymphoma, non-Hodgkin's lymphoma, skin cancer, a brain cancer, a cancer of the central nervous system, ovarian cancer, uterine cancer, stomach cancer, pancreatic cancer, esophageal cancer, kidney cancer, liver cancer, or a head and neck cancer.

In another embodiment, the cancer is metastatic cancer.

In yet another embodiment, the cancer is brain cancer or melanoma.

In one embodiment, the brain cancer is metastatic brain cancer or a glioma.

In one embodiment, the glioma is pilocytic astrocytoma, astrocytoma, anaplastic astrocytoma or glioblastoma multiforme.

In one embodiment, the cancer is homologous-recombination deficient, such as BRCA-1 or BRCA-2 deficient, or is deficient in one or more proteins of the Fanconi family. In one embodiment, the deficiency is caused by a genetic mutation. In another embodiment, the phenotype resulting from the deficiency is caused by abnormally low expression of BRCA-1 or BRCA-2 protein. In another embodiment, the phenotype resulting from the deficiency is caused by abnormally low expression of one or more proteins of the Fanconi family.

In still another embodiment, the subject in need of treatment has previously undergone or is presently undergoing treatment for cancer. The treatment includes, but is not limited to, chemotherapy, radiation therapy, surgery or immunotherapy, such as administration of a cancer vaccine.

The tetracyclic 1H-indeno[1,2-b]pyridine-2(5H)-one analogs are also useful for treating or preventing a cancer caused by a virus. Such viruses include human papilloma virus, which can lead to cervical cancer (see, e.g., Hernandez-Avila et al., Archives of Medical Research (1997) 28:265-271); Epstein-Barr virus (EBV), which can lead to lymphoma (see, e.g., Herrmann et al., J Pathol (2003) 199(2):140-5); hepatitis B or C virus, which can lead to liver carcinoma (see, e.g., El-Serag, J Clin Gastroenterol (2002) 35(5 Suppl 2):S72-8); human T cell leukemia virus (HTLV)-I, which can lead to T-cell leukemia (see e.g., Mortreux et al., Leukemia (2003) 17(1):26-38); human herpesvirus-8 infection, which can lead to Kaposi's sarcoma (see, e.g., Kadow et al., Curr Opin Investig Drugs (2002) 3(11):1574-9); and Human Immune deficiency Virus (HIV) infection, which can lead to cancer as a consequence of immunodeficiency (see, e.g., Dal Maso et al., Lancet Oncol (2003) 4(2):110-9).

### 4.2.10.1 Prophylactic Methods for Cancer

The tetracyclic 1H-indeno[1,2-b]pyridine-2(5H)-one analogs are also useful for preventing cancer prophylactically, or preventing progression of a cancer, including but not limited to the cancers listed in Table 1. Such prophylactic use includes that in which non-neoplastic cell growth such as hyperplasia, metaplasia, or most specifically, dysplasia has occurred.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a subject, can indicate the desirability of prophylactic or therapeutic administration of a tetracyclic 1H-indeno[1,2-b]pyridine-2(5H)-one analog. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, is treatable or preventable according to the present methods.

In another embodiment, fibrocystic disease (cystic hyperplasia, mammary dysplasia, specifically adenosis (benign epithelial hyperplasia)) is treatable or preventable according to the present methods.

In other embodiments, a subject that has one or more of the following predisposing factors for malignancy can be treated by administration of an effective amount of a tetracyclic 1H-indeno[1,2-b]pyridine-2(5H)-one analog: a chromosomal translocation associated with a malignancy (e.g., the Philadelphia chromosome for chronic myelogenous leukemia; t(14;18) for follicular lymphoma); familial polyposis or Gardner's syndrome; benign monoclonal gammopathy; a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome); and exposure to carcinogens (e.g., smoking, second-hand smoke exposure, and inhalation of or contacting with certain chemicals).

### 4.2.10.2 Combination Chemotherapy for the Treatment of Cancer

In one aspect, the present methods for treating or preventing cancer can further comprise the administration of another anticancer agent.

In one embodiment, the present invention provides methods for treating or preventing cancer, comprising the administration of an effective amount of a tetracyclic 1H-indeno[1,2-b]pyridine-2(5H)-one analog and another anticancer agent to a subject in need thereof.

The tetracyclic 1H-indeno[1,2-b]pyridine-2(5H)-one analog and another anticancer agent can be administered concurrently. In this embodiment, the tetracyclic 1H-indeno[1,2-b]pyridine-2(5H)-one analog and another anticancer agent can be administered within the same composition, or can be administered from different compositions, via the same or different routes of administration.

In another embodiment, the tetracyclic 1H-indeno[1,2-b]pyridine-2(5H)-one analog is administered during a time when the other anticancer agent exerts its prophylactic or therapeutic effect, or vice versa.

In another embodiment, the tetracyclic 1H-indeno[1,2-b]pyridine-2(5H)-one analog or other anticancer agent are administered in doses commonly employed when such agents are used as monotherapy for the treatment of cancer.

In one embodiment, the tetracyclic 1H-indeno[1,2-b]pyridine-2(5H)-one analog or other anticancer agent are administered in doses that are lower than the doses commonly employed when such agents are used as monotherapy for the treatment of cancer.

In another embodiment, the tetracyclic 1H-indeno[1,2-b]pyridine-2(5H)-one analog and other anticancer agent act synergistically and are administered in doses that are lower than the doses commonly employed when such agents are used as monotherapy for the treatment of cancer.

The dosage of the tetracyclic 1H-indeno[1,2-b]pyridine-2(5H)-one analog or other anticancer agent administered as well as the dosing schedule can depend on various parameters, including, but not limited to, the cancer being treated, the subject's general health, and the administering physician's discretion.

An Tetracyclic 1H-indeno[1,2-b]pyridine-2(5H)-one analog can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the other anticancer agent, to a subject in need thereof. In various embodiments a tetracyclic 1H-indeno[1,2-b]pyridine-2(5H)-one analog and the other anticancer agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, a tetracyclic 1H-indeno[1,2-b]pyridine-2(5H)-one analog and the other anticancer agent are administered within 3 hours. In another embodiment, a tetracyclic 1H-indeno[1,2-b]pyridine-2(5H)-one analog and the other anticancer agent are administered at 1 minute to 24 hours apart.

In one embodiment, an effective amount of a tetracyclic 1H-indeno[1,2-b]pyridine-2(5H)-one analog and an effective amount of other anticancer agent are present in the same composition. In one embodiment, this composition is useful for oral administration. In another embodiment, this composition is useful for intravenous administration.

In one embodiment, the compositions comprise an amount of a tetracyclic 1H-indeno[1,2-b]pyridine-2(5H)-one analog and the other anticancer agent which together are effective to treat or prevent cancer.

In another embodiment, the compositions comprise an effective amount of temozolomide, procarbazine, dacarbazine, interleukin-2, irinotecan, or doxorubicin, a physiologically acceptable carrier or vehicle, and an effective amount of a tetracyclic 1H-indeno[1,2-b]pyridine-2(5H)-one analog.

In one embodiment, the amount of a tetracyclic 1H-indeno[1,2-b]pyridine-2(5H)-one analog and the other anticancer agent is at least about 0.01% of the combined combination chemotherapy agents by weight of the composition. When intended for oral administration, this amount can be varied from about 0.1% to about 80% by weight of the composition. Some oral compositions can comprise from about 4% to about 50% of combined amount of a tetracyclic 1H-indeno[1,2-b]pyridine-2(5H)-one analog and the other anticancer agent by weight of the composition. Other compositions of the present invention are prepared so that a parenteral dosage unit contains from about 0.01% to about 2% by weight of the composition.

Cancers that can be treated or prevented by administering a tetracyclic 1H-indeno[1,2-b]pyridine-2(5H)-one analog and the other anticancer agent include, but are not limited to, the list of cancers set forth above in Table 1.

In one embodiment, the cancer is brain cancer.

In specific embodiments, the brain cancer is pilocytic astrocytoma, astrocytoma, anaplastic astrocytoma, glioblastoma multiforme or a metastatic brain tumor.

In one embodiment, the cancer is melanoma.

In a specific embodiment, the melanoma is metastatic melanoma.

The tetracyclic 1H-indeno[1,2-b]pyridine-2(5H)-one analog and other anticancer agent can act additively or synergistically. A synergistic combination of a tetracyclic 1H-indeno[1,2-b]pyridine-2(5H)-one analog and the other anticancer agent, might allow the use of lower dosages of one or both of these agents and/or less frequent administration of the agents to a subject with cancer. The ability to utilize lower dosages of one or both of the tetracyclic 1H-indeno[1,2-b]pyridine-2(5H)-one analog and other anticancer agent and/or to administer the agents less frequently can reduce any toxicity associated with the administration of the agents to a subject without reducing the efficacy of the agents in the treatment of cancer. In addition, a synergistic effect might result in the improved efficacy of these agents in the treatment of cancer and/or the reduction of any adverse or unwanted side effects associated with the use of either agent alone.

In one embodiment, the administration of an effective amount of a tetracyclic 1H-indeno[1,2-b]pyridine-2(5H)-one analog and an effective amount of another anticancer agent inhibits the resistance of a cancer to the other anticancer agent. In one embodiment, the cancer is a tumor.

Suitable other anticancer agents useful in the methods and compositions of the present invention include, but are not limited to temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

In one embodiment, the other anticancer agent is, but is not limited to, a drug listed in Table 2.

TABLE 2

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| | Procarbazine |
| | Temozolomide |
| Platinum containing complexes: | Cisplatin |
| | Carboplatin |
| | Aroplatin |
| | Oxaliplatin |
| Plant Alkaloids | |
| Vinca alkaloids: | Vincristine |
| | Vinblastine |
| | Vindesine |
| | Vinorelbine |
| Taxoids: | Paclitaxel |
| | Docetaxel |
| DNA Topoisomerase Inhibitors | |
| Epipodophyllins: | Etoposide |
| | Teniposide |
| | Topotecan |
| | 9-aminocamptothecin |
| | Camptothecin |
| | Crisnatol |
| Mitomycins: | Mitomycin C |
| | Anti-metabolites |
| Anti-folates: | |
| DHFR inhibitors: | Methotrexate |
| | Trimetrexate |
| IMP dehydrogenase Inhibitors: | Mycophenolic acid |
| | Tiazofurin |
| | Ribavirin |
| | EICAR |
| Ribonuclotide reductase | Hydroxyurea |
| Inhibitors: | Deferoxamine |
| Pyrimidine analogs: | |
| Uracil analogs: | 5-Fluorouracil |
| | Fluoxuridine |
| | Doxifluridine |
| | Ralitrexed |
| Cytosine analogs: | Cytarabine (ara C) |
| | Cytosine arabinoside |
| | Fludarabine |
| | Gemcitabine |
| | Capecitabine |
| Purine analogs: | Mercaptopurine |
| | Thioguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | Pyrazoloimidazole |
| Hormonal therapies: | |
| Receptor antagonists: | |
| Anti-estrogen: | Tamoxifen |
| | Raloxifene |
| | Megestrol |
| LHRH agonists: | Goscrclin |
| | Leuprolide acetate |
| Anti-androgens: | Flutamide |
| | Bicalutamide |
| Retinoids/Deltoids | |
| Vitamin A derivative: | Cis-retinoic acid |
| | All-trans retinoic acid (ATRA-IV) |
| Vitamin D3 analogs: | EB 1089 |
| | CB 1093 |
| | KH 1060 |
| Photodynamic therapies: | Vertoporfin (BPD-MA) |
| | Phthalocyanine |
| | Photosensitizer Pc4 |
| | Demethoxy-hypocrellin A |
| | (2BA-2-DMHA) |
| Cytokines: | Interferon-a |
| | Interferon-β |
| | Interferon-γ |
| | Tumor necrosis factor |
| | Interleukin-2 |
| Angiogenesis Inhibitors: | Angiostatin (plasminogen |
| | fragment) |
| | antiangiogenic antithrombin III |
| | Angiozyme |
| | ABT-627 |
| | Bay 12-9566 |
| | Benefin |
| | Bevacizumab |
| | BMS-275291 |
| | cartilage-derived inhibitor (CDI) |
| | CAI |
| | CD59 complement fragment |
| | CEP-7055 |
| | Col 3 |
| | Combretastatin A-4 |
| | Endostatin (collagen XVIII |
| | fragment) |
| | Fibronectin fragment |
| | Gro-beta |
| | Halofuginone |
| | Heparinases |
| | Heparin hexasaccharide fragment |
| | HMV833 |
| | Human chorionic gonadotropin |
| | (hCG) |
| | IM-862 |
| | Interferon alpha/beta/gamma |
| | Interferon inducible protein (IP- |
| | 10) |
| | Interleukin-12 |
| | Kringle 5 (plasminogen fragment) |
| | Marimastat |
| | Metalloproteinase inhibitors |
| | (TIMPs) |
| | 2-Methoxyestradiol |
| | MMI 270 (CGS 27023A) |
| | MoAb IMC-1C11 |
| | Neovastat |
| | NM-3 |
| | Panzem |
| | PI-88 |
| | Placental ribonuclease inhibitor |
| | Plasminogen activator inhibitor |
| | Platelet factor-4 (PF4) |
| | Prinomastat |
| | Prolactin 16 kD fragment |
| | Proliferin-related protein (PRP) |
| | PTK 787/ZK 222594 |
| | Retinoids |
| | Solimastat |
| | Squalamine |
| | SS 3304 |
| | SU 5416 |
| | SU6668 |
| | SU11248 |
| | Tetrahydrocortisol-S |
| | Tetrathiomolybdate |
| | Thalidomide |
| | Thrombospondin-1 (TSP-1) |
| | TNP-470 |
| | Transforming growth factor-beta |
| | (TGF-β) |
| | Vasculostatin |
| | Vasostatin (calreticulin fragment) |
| | ZD6126 |
| | ZD 6474 |
| | farnesyl transferase inhibitors |
| | (FTI) |
| | Bisphosphonates |
| Antimitotic agents: | Allocolchicine |
| | Halichondrin B |
| | Colchicine |
| | colchicine derivative |
| | dolstatin 10 |
| | Maytansine |
| | Rhizoxin |
| | Thiocolchicine |
| | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin (adriamycin) |
| | Idarubicin |
| | Epirubicin |
| | Pirarubicin |
| | Zorubicin |
| | Mitoxantrone |
| MDR inhibitors: | Verapamil |
| Ca²⁺ATPase inhibitors: | Thapsigargin |

Other additional anticancer agents that are useful in the compositions and methods of the present invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine hydrochloride; hydroxyurea; idarubicin hydrochloride; ifosfamide; ilmofosine; interleukin-2 (including recombinant interleukin-2, or rIL2), interferon alfa-2α; interferon alfa-2β; interferon alfa-n1; interferon alfa-n3; interferon beta-Iα; interferon gamma-I β; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Further anticancer drugs that are useful in the methods and compositions of the invention include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta Lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-acytidine; dihydrotaxol; dioxamycin; diphenyl spiromustine; docetaxel; docosanol; dolasetron; doxifluridine; droloxifene; dronabinol; duocaimycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum complexes; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; initonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agents; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum complexes; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In one another embodiment, the other anticancer agent is interferon-a.

In another embodiment, the other anticancer agent is interleukin-2.

In one embodiment, the other anticancer agent is an alkylating agent, such as a nitrogen mustard, a nitrosourea, an alkylsulfonate, a triazene, or a platinum-containing agent.

In one embodiment, the other anticancer agent is a triazene alkylating agent.

In another embodiment, the other anticancer agent is temozolomide, procarbazine, dacarbazine, interleukin-2, irinotecan, doxorubicin, or a combination thereof.

In a specific embodiment, the other anticancer agent is temozolomide.

Temozolomide can be administered to a subject at dosages ranging from about 60 mg/m² (of a subject's body surface area) to about 250 mg/m² and from about 100 mg/m² to about 200 mg/m². In specific embodiments, the dosages of temozolomide are about 10 mg/m², about 1 mg/m², about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², or about 250 mg/m².

In a specific embodiment, temozolomide is administered orally.

In one embodiment, temozolomide is administered orally to a subject at a dose ranging from about 150 mg/m² to about 200 mg/m².

In another embodiment, temozolomide is administered orally to a subject once per day for five consecutive days at a dose ranging from about 150 mg/m² to about 200 mg/m².

In a specific embodiment, temozolomide is administered orally to a subject once per day for five consecutive days at a dose ranging from about 150 mg/m² to about 200 mg/m² on days 1-5, then again orally once per day for five consecutive days on days 28-32 at a dose ranging from about 150 mg/m² to about 200 mg/m², then again orally once per day for five consecutive days on days 55-59 at a dose ranging from about 150 mg/m² to about 200 mg/m².

In another embodiment, temozolomide is administered orally to a subject once per day for a week, two weeks, three weeks, a month or longer at the foregoing daily dosage.

In a specific embodiment, the other anticancer agent is procarbazine.

Procarbazine can be administered to a subject at dosages ranging from about 50 mg/m² (of a subject's body surface area) to about 100 mg/m² and from about 60 mg/m² to about 100 mg/m². In specific embodiments, the dosages of procarbazine are about 10 mg/m², about 1 mg/m², about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 g/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², about 250 mg/m², about 260 mg/m², about 270 mg/m², about 280 mg/m², about 290 mg/m², about 300 mg/m², about 310 mg/m², about 320 mg/m², about 330 mg/m², about 340 mg/m², about 350 mg/m², about 360 mg/m², about 370 mg/m², about 380 mg/m², about 390 mg/m², about 400 mg/m², about 410 mg/m², about 420 mg/m², about 430 mg/m², about 440 mg/m², about 450 mg/m², about 460 mg/m², about 470 mg/m², about 480 mg/m², about 490 mg/m², or about 500 mg/m².

In a specific embodiment, procarbazine is administered intravenously.

In one embodiment, procarbazine is administered intravenously to a subject at a dose ranging from about 50 mg/m² to about 100 mg/m².

In another embodiment, procarbazine is administered intravenously to a subject once per day for five consecutive days at a dose ranging from about 50 mg/m² to about 100 mg/m².

In a specific embodiment, procarbazine is administered intravenously to a subject once per day for five consecutive days at a dose ranging from about 50 mg/m² to about 100 mg/m² on days 1-5, then again intravenously once per day for five consecutive days on days 28-32 at a dose ranging from about 50 mg/m² to about 100 mg/m², then again intravenously once per day for five consecutive days on days 55-59 at a dose ranging from about 50 mg/m² to about 100 mg/m².

In another embodiment, procarbazine is administered once intravenously to a subject at a dose ranging from about 50 m g/m² to about 100 mg/m².

In another embodiment, procarbazine is administered intravenously to a subject once per day for a week, two weeks, three weeks, a month or longer at the foregoing daily dosage.

In a specific embodiment, the other anticancer agent is dacarbazine.

Dacarbazine can be administered to a subject at dosages ranging from about 60 mg/m² (of a subject's body surface area) to about 250 mg/m² and from about 150 mg/m² to about 250 mg/m². In specific embodiments, the dosages of dacarbazine are about 10 mg/m², about 1 mg/m², about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², about 250 mg/m², about 260 mg/m², about 270 mg/m², about 280 mg/m², about 290 mg/m², about 300 mg/m², about 310 mg/m², about 320 mg/m², about 330 mg/m², about 340 mg/m², about 350 mg/m², about 360 mg/m², about 370 mg/m², about 380 mg/m², about 390 mg/m², about 400 mg/m², about 410 mg/m², about 420 mg/m², about 430 mg/m², about 440 mg/m², about 450 mg/m², about 460 mg/m², about 470 mg/m², about 480 mg/m², about 490 mg/m², or about 500 mg/m²

In a specific embodiment, dacarbazine is administered intravenously.

In one embodiment, dacarbazine is administered intravenously to a subject at a dose ranging from about 150 mg/m² to about 250 mg/m².

In another embodiment, dacarbazine is administered intravenously to a subject once per day for five consecutive days at a dose ranging from about 150 mg/m² to about 250 mg/m².

In a specific embodiment, dacarbazine is administered intravenously to a subject once per day for five consecutive days at a dose ranging from about 150 mg/m² to about 250 mg/m² on days 1-5, then again intravenously once per day for five consecutive days on days 28-32 at a dose ranging from about 150 mg/m² to about 250 mg/m², then again intravenously once per day for five consecutive days on days 55-59 at a dose ranging from about 150 mg/m² to about 250 mg/m².

In one embodiment, dacarbazine is administered once intravenously to a subject at a dose ranging from about 150 mg/m² to about 250 mg/m².

In another embodiment, dacarbazine is administered intravenously to a subject once per day for a week, two weeks, three weeks, a month or longer at the foregoing daily dosage.

In a specific embodiment, the other anticancer agent is doxorubicin.

Doxorubicin can be administered to a subject at dosages ranging from about 50 mg/m² (of a subject's body surface area) to about 100 mg/m² and from about 60 mg/m² to about 100 mg/m². In specific embodiments, the dosages of doxorubicin are about 10 mg/m², about 1 mg/m², about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², about 250 mg/m², about 260 mg/m², about 270 mg/m², about 280 mg/m², about 290 mg/m², about 300 mg/m², about 310 mg/m², about 320 mg/m², about 330 mg/m², about 340 mg/m², about 350 mg/m², about 360 mg/m², about 370 mg/m², about 380 mg/m², about 390 mg/m², about 400 mg/m², about 410 mg/m², about 420 mg/m², about 430 mg/m², about 440 mg/m², about 450 mg/m², about 460 mg/m², about 470 mg/m², about 480 mg/m², about 490 mg/m², or about 500 mg/m².

In a specific embodiment, doxorubicin is administered intravenously.

In one embodiment, doxorubicin is administered intravenously to a subject at a dose ranging from about 50 mg/m² to about 100 mg/m².

In another embodiment, doxorubicin is administered intravenously to a subject once per day for five consecutive days at a dose ranging from about 50 mg/m² to about 100 mg/m².

In a specific embodiment, doxorubicin is administered intravenously to a subject once per day for five consecutive days at a dose ranging from about 50 mg/m² to about 100 mg/m² on days 1-5, then again intravenously once per day for five consecutive days on days 28-32 at a dose ranging from about 50 mg/m² to about 100 mg/m², then again intravenously once per day for five consecutive days on days 55-59 at a dose ranging from about 50 mg/m² to about 100 mg/m².

In another embodiment, doxorubicin is administered once intravenously to a subject at a dose ranging from about 50 mg/m² to about 100 mg/m².

In another embodiment, doxorubicin is administered intravenously to a subject once per day for a week, two weeks, three weeks, a month or longer at the foregoing daily dosage.

In one embodiment, the other anticancer agent is a Topoisomerase I inhibitor, such as etoposide, teniposide, topotecan, irinotecan, 9-aminocamptothecin, camptothecin, or crisnatol.

In a specific embodiment, the other anticancer agent is irinotecan.

Irinotecan can be administered to a subject at dosages ranging from about 50 mg/m² (of a subject's body surface area) to about 150 mg/m² and from about 75 mg/m² to about 150 mg/m². In specific embodiments, the dosages of irinotecan are about 10 mg/m², about 1 mg/m², about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², about 250 mg/m², about 260 mg/m², about 270 mg/m², about 280 mg/m², about 290 mg/m², about 300 mg/m², about 310 mg/m², about 320 mg/m², about 330 mg/m², about 340 mg/m², about 350 mg/m², about 360 mg/m², about 370 mg/m², about 380 mg/m², about 390 mg/m², about 400 mg/m², about 410 mg/m², about 420 mg/m², about 430 mg/m², about 440 mg/m², about 450 mg/m², about 460 mg/m², about 470 mg/m², about 480 mg/m², about 490 mg/m², or about 500 mg/m².

In a specific embodiment, irinotecan is administered intravenously.

In one embodiment, irinotecan is administered intravenously to a subject at a dose ranging from about 50 mg/m² to about 150 mg/m².

In another embodiment, irinotecan is administered intravenously to a subject once per day for five consecutive days at a dose ranging from about 50 mg/m² to about 150 mg/m².

In a specific embodiment, irinotecan is administered intravenously to a subject once per day for five consecutive days at a dose ranging from about 50 mg/m² to about 150 mg/m² on days 1-5, then again intravenously once per day for five consecutive days on days 28-32 at a dose ranging from about 50 mg/m² to about 150 mg/m², then again intravenously once per day for five consecutive days on days 55-59 at a dose ranging from about 50 mg/m² to about 150 mg/m².

In another embodiment, irinotecan is administered intravenously to a subject once per day for a week, two weeks, three weeks, a month or longer at the foregoing daily dosage.

In one embodiment, the other anticancer agent is O-6-benzylguanine.

In another embodiment, the other anticancer agent is O-6-benzylguanine and temozolomide.

In another embodiment, the other anticancer agent is O-6-benzylguanine and procarbazine.

In still another embodiment, the other anticancer agent is O-6-benzylguanine and dacarbazine.

### 4.2.10.3 Multi-Therapy For Cancer

The tetracyclic 1H-indeno[1,2-b]pyridine-2(5H)-one analogs can be administered to a subject that has undergone or is currently undergoing one or more additional anticancer therapies including, but not limited to, surgery, radiation therapy, or immunotherapy, such as cancer vaccines.

In one embodiment, the invention provides methods for treating or preventing cancer comprising administering to a subject in need thereof an effective amount of a tetracyclic 1H-indeno[1,2-b]pyridine-2(5H)-one analog to treat or prevent cancer and another anticancer therapy including, but not limited to, surgery, radiation therapy, or immunotherapy, such as a cancer vaccine.

In one embodiment, the other anticancer therapy is radiation therapy.

In another embodiment, the other anticancer therapy is surgery.

In still another embodiment, the other anticancer therapy is immunotherapy.

In a specific embodiment, the present methods for treating or preventing cancer comprise administering an effective amount of a tetracyclic 1H-indeno[1,2-b]pyridine-2(5H)-one analog and radiation therapy. The radiation therapy can be administered concurrently with, prior to, or subsequent to the tetracyclic 1H-indeno[1,2-b]pyridine-2(5H)-one analog, in one embodiment at least an hour, five hours, 12 hours, a day, a week, a month, in another embodiment several months (e.g., up to three months), prior or subsequent to administration of the tetracyclic 1H-indeno[1,2-b]pyridine-2(5H)-one analog.

Where the other anticancer therapy is radiation therapy, any radiation therapy protocol can be administered depending upon the type of cancer to be treated. For example, but not by way of limitation, X-ray radiation can be administered; specifically, high-energy megavoltage (radiation of greater that 1 MeV energy) can be administered for deep tumors, and electron beam and orthovoltage X-ray radiation can be administered for skin cancers. Gamma-ray emitting radioisotopes, such as radioactive isotopes of radium, cobalt and other elements, can also be administered.

Additionally, the invention provides methods of treatment of cancer comprising administering a tetracyclic 1H-indeno[1,2-b]pyridine-2(5H)-one analog as an alternative to chemotherapy or radiation therapy where the chemotherapy or the radiation therapy results in a negative side effect in the subject being treated. The subject being treated can, optionally, be treated with another anticancer therapy such as surgery, radiation therapy, or immunotherapy.

The tetracyclic 1H-indeno[1,2-b]pyridine-2(5H)-one analogs can also be administered in vitro or ex vivo, such as for the treatment of certain cancers, including, but not limited to leukemias and lymphomas, such treatment involving autologous stem cell transplants. This can involve a process in which the subject's autologous hematopoietic stem cells are harvested and purged of all cancer cells, the subject's remaining bone-marrow cell population is then eradicated via the administration of a tetracyclic 1H-indeno[1,2-b]pyridine-2(5H)-one analog and/or radiation, and the resultant stem cells are infused back into the subject. Supportive care can be subsequently provided while bone marrow function is restored and the subject recovers.

### 4.2.11 Treatment or Prevention of Erectile Dysfunction

The tetracyclic 1H-indeno[1,2-b]pyridine-2(5H)-one analogs are useful for treating or preventing erectile dysfunction. Erectile dysfunction includes an inability to achieve or maintain a full erection, specifically that which is sufficient to achieve or maintain sexual intercourse. The inability can be a total inability, an inconsistent ability, or a tendency to maintain only a brief erection. Erectile dysfunction includes idiopathic erectile dysfunction, as well as that which can result, for example, from trauma, including mechanical trauma, specifically that resulting from surgery, to the nerves (such as during prostatectomy); diabetes mellitus; a cardiovascular disease, including atherosclerosis; radiation; or certain drugs. The erectile dysfunction can also be age-related.

In one embodiment, the erectile dysfunction results from prostate surgery.

In a further embodiment, the erectile dysfunction results from prostate nerve injury.

### 4.2.12 Treatment or Prevention of Urinary Incontinence

The tetracyclic 1H-indeno[1,2-b]pyridine-2(5H)-one analogs are also useful for treating or preventing urinary incontinence. Urinary incontinence can result, for example, from trauma, including mechanical trauma, specifically during childbirth or that resulting from surgery, to the nerves (such as during prostatectomy or gynecological surgery); diabetes mellitus; a cardiovascular disease, including atherosclerosis; radiation; or certain drugs. The urinary incontinence can also be age-related.

In one embodiment, the subject in need of urinary incontinence treatment or prevention is male.

In one embodiment, the subject in need of urinary incontinence treatment or prevention is female.

### 4.2.13 Treatment or Prevention of a Complication of Prematurity

The tetracyclic 1H-indeno[1,2-b]pyridine-2(5H)-one analogs are also useful for treating or preventing a complication of prematurity. Examples of complications of prematurity include, but are not limited to, retinopathy, hyaline-membrane disease and necrotizing enterocolitis.

### 4.2.14 Treatment or Prevention of Cardiomyopathy

The tetracyclic 1H-indeno[1,2-b]pyridine-2(5H)-one analogs are also useful for treating or preventing cardiomyopathy.

### 4.2.15 Treatment or Prevention of Retinopathy

The tetracyclic 1H-indeno[1,2-b]pyridine-2(5H)-one analogs are also useful for treating or preventing retinal degenerative diseases such as retinopathies, including diabetic retinopathy, hypertensive retinopathy, aged macular degeneration, retinopathy of prematurity, and macular oedema.

### 4.2.16 Treatment or Prevention of Nephropathy

The tetracyclic 1H-indeno[1,2-b]pyridine-2(5H)-one analogs are also useful for treating or preventing nephropathy, including contrast induced nephropathy.

### 4.2.17 Treatment or Prevention of Neuropathy

The tetracyclic 1H-indeno[1,2-b]pyridine-2(5H)-one analogs are also useful for treating or preventing neuropathy.

### 4.2.18 Treatment or Prevention of Ocular Angiogenesis and Ocular Neovascular Diseases

The tetracyclic 1H-indeno[1,2-b]pyridine-2(5H)-one analogs are also useful for treating or preventing ocular angiogenesis and ocular neovascular diseases such as corneal neovascularisation.

### 4.2.19 Treatment or Prevention of an Ocular Condition or Disease

The tetracyclic 1H-indeno[1,2-b]pyridine-2(5H)-one analogs are also useful for treating or preventing an ocular disease or condition where the inhibition of poly (ADP)-ribose synthase activity in the ocular environment is required. The ocular condition or disease includes aged macular degeneration, ocular inflammatory diseases or conditions including corneal dystrophy, trachoma, onchocerciasis, uveitis, sympathetic ophthalmitis and endophthalmitis; uveitis, retinitis, cytomegalovirus retinitis, also known as CMV retinitis, blepharitis, conjunctivitis, scleritistemporal arteritis, scleritis, conjunctivitis, keratitis, iritis, iridocyclitis, and progressive outer retinal necrosis (PORN), also known as Varicella zoster virus retinitis (VZVR) or a chronic and sub-clinical inflammatory disease such as glaucoma, aged macular degeneration or diabetic retinopathy; ocular retinopathies including diabetic retinopathy, hypertensive retinopathy and retinopathy of prematurity; glaucoma or a glaucoma related condition, ocular angiogenesis an ocular neovascular disease.

### 4.3 Therapeutic/Prophylactic Administration and Compositions of the Invention

Due to their activity, the tetracyclic 1H-indeno[1,2-b]pyridine-2(5H)-one analogs are advantageously useful in veterinary and human medicine. As described above, the tetracyclic 1H-indeno[1,2-b]pyridine-2(5H)-one analogs are useful for treating or preventing a Condition in a subject in need thereof.

The tetracyclic 1H-indeno[1,2-b]pyridine-2(5H)-one analogs can be administered in amounts that are effective to treat or prevent a Condition in a subject in need thereof.

When administered to a subject, the tetracyclic 1H-indeno[1,2-b]pyridine-2(5H)-one analogs can be administered as a component of a composition that comprises a physiologically acceptable carrier or vehicle. The present compositions, which comprise a tetracyclic 1H-indeno[1,2-b]pyridine-2(5H)-one analog, can be administered orally. The tetracyclic 1H-indeno[1,2-b]pyridine-2(5H)-one analogs can also be administered by any other convenient route, for example, by infusion or bolus injection, by absorption through epithelial or mucocutaneous linings (e.g., oral, rectal, or intestinal mucosa) and can be administered together with another biologically active agent. Administration can be systemic or local. Various delivery systems are known, e.g., encapsulation in liposomes, microparticles, microcapsules and capsules.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, specifically to the ears, nose, eyes, or skin. In some instances, administration will result in the release of a tetracyclic 1H-indeno[1,2-b]pyridine-2(5H)-one analog into the bloodstream.

In one embodiment, the tetracyclic 1H-indeno[1,2-b]pyridine-2(5H)-one analogs are administered orally.

In other embodiments, it can be desirable to administer the tetracyclic 1H-indeno[1,2-b]pyridine-2(5H)-one analogs locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the tetracyclic 1H-indeno[1,2-b]pyridine-2(5H)-one analogs into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon oar, synthetic pulmonary surfactant. In certain embodiments, the tetracyclic 1H-indeno[1,2-b]pyridine-2(5H)-one analogs can be formulated as a suppository, with traditional binders and excipients such as triglycerides.

In another embodiment Tetracyclic 1H-indeno[1,2-b]pyridine-2(5H)-one analogs can be delivered in a vesicle, specifically a liposome (see Langer, Science 249:1527-1533 (1990) and Treat or prevent et al., Liposomes in Therapy of Infectious Disease and Cancer 317-327 and 353-365 (1989)).

In yet another embodiment, the tetracyclic 1H-indeno[1,2-b]pyridine-2(5H)-one analogs can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment a controlled- or sustained-release system can be placed in proximity of a target of the tetracyclic 1H-indeno[1,2-b]pyridine-2(5H)-one analogs, e.g., the spinal column, brain, skin, lung, or gastrointestinal tract, thus requiring only a fraction of the systemic dose.

The present compositions can optionally comprise a suitable amount of a pharmaceutically acceptable excipient so as to provide the form for proper administration to the subject.

Such pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when the tetracyclic 1H-indeno[1,2-b]pyridine-2(5H)-one analog is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions. aerosols, sprays, suspensions, or any other form suitable for use. In one embodiment, the composition is in the form of a capsule (see e.g. U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In one embodiment, the tetracyclic 1H-indeno[1,2-b]pyridine-2(5H)-one analog is formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving a tetracyclic 1H-indeno[1,2-b]pyridine-2(5H)-one analog are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the tetracyclic 1H-indeno[1,2-b]pyridine-2(5H)-one analogs can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized-powder or water-free concentrate in a hermetically sealed container such as an ampule or sachette indicating the quantity of active agent. Where the tetracyclic 1H-indeno[1,2-b]pyridine-2(5H)-one analogs are to be administered by infusion, they can be dispensed, for example, with an infusion bottle containing sterile pharmaceutical grade water or saline. Where the tetracyclic 1H-indeno[1,2-b]pyridine-2(5H)-one analogs are administered by injection, an ampule of sterile water for injection or saline can be provided so that the ingredients can be mixed prior to administration.

Tetracyclic 1H-indeno[1,2-b]pyridine-2(5H)-one analogs can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of a tetracyclic 1H-indeno[1,2-b]pyridine-2(5H)-one analog to treat or prevent the Condition over a period of time. Advantages of controlled- or sustained-release compositions include extended activity of the drug, reduced dosage frequency, and increased subject compliance. In addition, controlled- or sustained-release compositions can favorably affect the time of onset of action or other characteristics, such as blood levels of the tetracyclic 1H-indeno[1,2-b]pyridine-2(5H)-one analog, and can thus reduce the occurrence of adverse side effects.

Controlled- or sustained-release compositions can initially release an amount of a tetracyclic 1H-indeno[1,2-b]pyridine-2(5H)-one analog that promptly produces the desired therapeutic or prophylactic effect, and gradually and continually release other amounts of the tetracyclic 1H-indeno[1,2-b]pyridine-2(5H)-one analog to maintain this level of therapeutic or prophylactic effect over an extended period of time. To maintain a constant level of the tetracyclic 1H-indeno[1,2-b]pyridine-2(5H)-one analog in the body, the tetracyclic 1H-indeno[1,2-b]pyridine-2(5H)-one analog can be released from the dosage form at a rate that will replace the amount of Tetracyclic 1H-indeno[1,2-b]pyridine-2(5H)-one analog being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

The amount of the tetracyclic 1H-indeno[1,2-b]pyridine-2(5H)-one analog that is effective in the treatment or prevention of a Condition can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of the practitioner and each subject's circumstances in view of, e.g., published clinical studies. Suitable effective dosage amounts, however, range from about 10 micrograms to about 5 grams about every 4 hours, although they are typically about 500 mg or less per every 4 hours. In one embodiment, the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one Tetracyclic 1H-indeno[1,2-b]pyridine-2(5H)-one analog is administered, the effective dosage amounts correspond to the total amount administered.

Compositions can be prepared according to conventional mixing, granulating or coating methods, respectively, and the present compositions can contain, in one embodiment, from about 0.1% to about 99%; and in another embodiment from about 1% to about 70% of the tetracyclic 1H-indeno[1,2-b]pyridine-2(5H)-one analog by weight or volume.

The dosage regimen utilizing the tetracyclic 1H-indeno[1,2-b]pyridine-2(5H)-one analog can be selected in accordance with a variety of factors including type, species, age, weight, sex and medical condition of the subject; the severity of the condition to be treated; the route of administration; the renal or hepatic function of the subject; and the specific Tetracyclic 1H-indeno[1,2-b]pyridine-2(5H)-one analog employed. A person skilled in the art can readily determine the effective amount of the drug useful for treating or preventing the Condition.

An Tetracyclic 1H-indeno[1,2-b]pyridine-2(5H)-one analog can be administered in a single daily dose, or the total daily dosage can be administered in divided doses of two, three or four times daily. Furthermore, a tetracyclic 1H-indeno[1,2-b]pyridine-2(5H)-one analog can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of Tetracyclic 1H-indeno[1,2-b]pyridine-2(5H)-one analog ranges from about 0.1% to about 15%, w/w or w/v.

The tetracyclic 1H-indeno[1,2-b]pyridine-2(5H)-one analogs can be assayed in vitro or in vivo for the desired therapeutic or prophylactic activity prior to use in humans. Animal model systems can be used to demonstrate safety and efficacy.

The present methods for treating or preventing a Condition in a subject in need thereof can further comprise administering another prophylactic or therapeutic agent to the subject being administered a tetracyclic 1H-indeno[1,2-b]pyridine-2(5H)-one analog. In one embodiment, the other prophylactic or therapeutic agent is administered in an effective amount. The other prophylactic or therapeutic agent includes, but is not limited to, an anti-inflammatory agent, an anti-renal failure agent, an anti-diabetic agent, and anti-cardiovasculare disease agent, an antiemetic agent, a hematopoietic colony stimulating factor, an anxiolytic agent, and an analgesic agent.

In one embodiment, the other prophylactic or therapeutic agent is an agent useful for reducing any potential side effect of a tetracyclic 1H-indeno[1,2-b]pyridine-2(5H)-one analog. Such potential side effects include, but are not limited to, nausea, vomiting, headache, low white blood cell count, low red blood cell count, low platelet count, headache, fever, lethargy, a muscle ache, general pain, bone pain, pain at an injection site, diarrhea, neuropathy, pruritis, a mouth sore, alopecia, anxiety or depression.

In one embodiment, the tetracyclic 1H-indeno[1,2-b]pyridine-2(5H)-one analog can be administered prior to, concurrently with, or after an anti-inflammatory agent, or on the same day, or within 1 hour, 2 hours, 12 hours, 24 hours, 48 hours or 72 hours of each other.

In another embodiment, the tetracyclic 1H-indeno[1,2-b]pyridine-2(5H)-one analog can be administered prior to, concurrently with, or after an anti-renal failure agent, or on the same day, or within 1 hour, 2 hours, 12 hours, 24 hours, 48 hours or 72 hours of each other.

In still another embodiment, the tetracyclic 1H-indeno[1,2-b]pyridine-2(5H)-one analog can be administered prior to, concurrently with, or after an anti-diabetic agent, or on the same day, or within 1 hour, 2 hours, 12 hours, 24 hours, 48 hours or 72 hours of each other.

In yet another embodiment, the tetracyclic 1H-indeno[1,2-b]pyridine-2(5H)-one analog can be administered prior to, concurrently with, or after an anti-cardiovascular disease agent, or on the same day, or within 1 hour, 2 hours, 12 hours, 24 hours, 48 hours or 72 hours of each other.

In a further embodiment, the tetracyclic 1H-indeno[1,2-b]pyridine-2(5H)-one analog can be administered prior to, concurrently with, or after an antiemetic agent, or on the same day, or within 1 hour, 2 hours, 12 hours, 24 hours, 48 hours or 72 hours of each other.

In another embodiment, the tetracyclic 1H-indeno[1,2-b]pyridine-2(5H)-one analog can be administered prior to, concurrently with, or after a hematopoietic colony stimulating factor, or on the same day, or within 1 hour, 2 hours, 12 hours, 24 hours, 48 hours, 72 hours, 1 week, 2 weeks, 3 weeks or 4 weeks of each other.

In still embodiment, the tetracyclic 1H-indeno[1,2-b]pyridine-2(5H)-one analog can be administered prior to, concurrently with, or after an opioid or non-opioid analgesic agent, or on the same day, or within 1 hour, 2 hours, 12 hours, 24 hours, 48 hours or 72 hours of each other.

In yet another embodiment, the tetracyclic 1H-indeno[1,2-b]pyridine-2(5H)-one analog can be administered prior to, concurrently with, or after an anxiolytic agent, or on the same day, or within 1 hour, 2 hours, 12 hours, 24 hours, 48 hours or 72 hours of each other.

Effective amounts of the other prophylactic or therapeutic agents are well known to those skilled in the art. However, it is well within the skilled artisan's purview to determine the other prophylactic or therapeutic agent's optimal effective amount range. In one embodiment of the invention, where another prophylactic or therapeutic agent is administered to a subject, the effective amount of the tetracyclic 1H-indeno[1,2-b]pyridine-2(5H)-one analog is less than its effective amount would be where the other prophylactic or therapeutic agent is not administered. In this case, without being bound by theory, it is believed that Tetracyclic 1H-indeno[1,2-b]pyridine-2(5H)-one analogs and the other prophylactic or therapeutic agent act synergistically to treat or prevent a Condition.

In one embodiment, the other therapeutic or prophylactic agent is an anti-inflammatory agent. Anti-inflammatory agents useful in the methods of the present invention include but are not limited to adrenocorticosteroids, such as cortisol, cortisone, fludrocortisone, prednisone, prednisolone, 6α-methylprednisolone, triamcinolone, betamethasone, and dexamethasone; and non-steroidal anti-inflammatory agents (NSAIDs), such as aspirin, acetaminophen, indomethacin, sulindac, tolmetin, diclofenac, ketorolac, ibuprofen, naproxen, flurbiprofen, ketoprofen, fenoprofen, oxaprozin, mefenamic acid, meclofenamic acid, piroxicam, meloxicam, nabumetone, rofecoxib, celecoxib, etodolac, and nimesulide.

In one embodiment, the other therapeutic or prophylactic agent is an anti-renal failure agent. Anti-renal failure agents useful in the methods of the present invention include but are not limited to ACE (angiotensin-converting enzyme) inhibitors, such as captopril, enalaprilat, lisinopril, benazepril, fosinopril, trandolapril, quinapril, and ramipril; diuretics, such as mannitol, glycerin, furosemide, toresemide, tripamide, chlorothiazide, methyclothiazide, indapamide, amiloride, and spironolactone; and fibric acid agents, such as clofibrate, gemfibrozil, fenofibrate, ciprofibrate, and bezafibrate.

In one embodiment, the other therapeutic or prophylactic agent is an anti-diabetic agent. Anti-diabetic agents useful in the methods of the present invention include but are, not limited to glucagons; somatostatin; diazoxide; sulfonylureas, such as tolbutamide, acetohexamide, tolazamide, chloropropamide, glybenclamide, glipizide, gliclazide, and glimepiride; insulin secretagogues, such as repaglinide, and nateglinide; biguanides, such as metformin and phenformin; thiazolidinediones, such as pioglitazone, rosiglitazone, and troglitazone; and α-glucosidase inhibitors, such as acarbose and miglitol.

In one embodiment, the other therapeutic or prophylactic agent is an anti-cardiovascular agent. Anti-cardiovascular disease agents useful in the methods of the present invention include but are not limited to carnitine; thiamine; and muscarinic receptor antagonists, such as atropine, scopolamine, homatropine, tropicamide, pirenzipine, ipratropium, tiotropium, and tolterodine.

In one embodiment, the other therapeutic or prophylactic agent is an antiemetic agent. Antiemetic agents useful in the methods of the present invention include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, and mixtures thereof.

In one embodiment, the other therapeutic or prophylactic agent is a hematopoietic colony stimulating factor. Hematopoietic colony stimulating factors useful in the methods of the present invention include, but are not limited to, filgrastim, sargramostim, molgramostim and epoietin alfa.

In one embodiment, the other therapeutic or prophylactic agent is an opioid analgesic agent. Opioid analgesic agents useful in the methods of the present invention include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene.

In one embodiment, the other therapeutic or prophylactic agent is a non-opioid analgesic agent. Non-opioid analgesic agents useful in the methods of the present invention include, but are not limited to, aspirin, celecoxib, rofecoxib, diclofenac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In one embodiment, the other therapeutic or prophylactic agent is an anxiolytic agent. Anxiolytic agents useful in the methods of the present invention include, but are not limited to, buspirone, and benzodiazepines such as diazepam, lorazepam, oxazapam, chlorazepatc, clonazepam, chlordiazepoxide and alprazolam.

### 4.4 Kits

The invention encompasses kits that can simplify the administration of a tetracyclic 1H-indeno[1,2-b]pyridine-2(5H)-one analog to a subject.

A typical kit of the invention comprises a unit dosage form of a tetracyclic 1H-indeno[1,2-b]pyridine-2(5H)-one analog. In one embodiment, the unit dosage form is a container, which can be sterile, containing an effective amount of a tetracyclic 1H-indeno[1,2-b]pyridine-2(5H)-one analog and a physiologically acceptable carrier or vehicle. The kit can further comprise a label or printed instructions instructing the use of the tetracyclic 1H-indeno[1,2-b]pyridine-2(5H)-one analog to treat or prevent a Condition. The kit can also further comprise a unit dosage form of another prophylactic or therapeutic agent, for example, a container containing an effective amount of the other prophylactic or therapeutic agent. In one embodiment, the kit comprises a container containing an effective amount of a tetracyclic 1H-indeno[1,2-b]pyridine-2(5H)-one analog and an effective amount of another prophylactic or therapeutic agent. Examples of other prophylactic or therapeutic agents include, but are not limited to, those listed above.

The invention is further described in the following examples, which do not limit the scope of the invention described in the claims.

### 5. EXAMPLES AND EXPERIMENTAL

### 5.1 Methods For Making Tetracyclic 1H-indeno[1,2-b]pyridine-2(5H)-one analogs

Methods useful for making the tetracyclic 1H-indeno[1,2-b]pyridine-2(5H)-one analogs are set forth in the following Experiment and Examples described below and as depicted schematically in the following generalized Schemes 1-7.

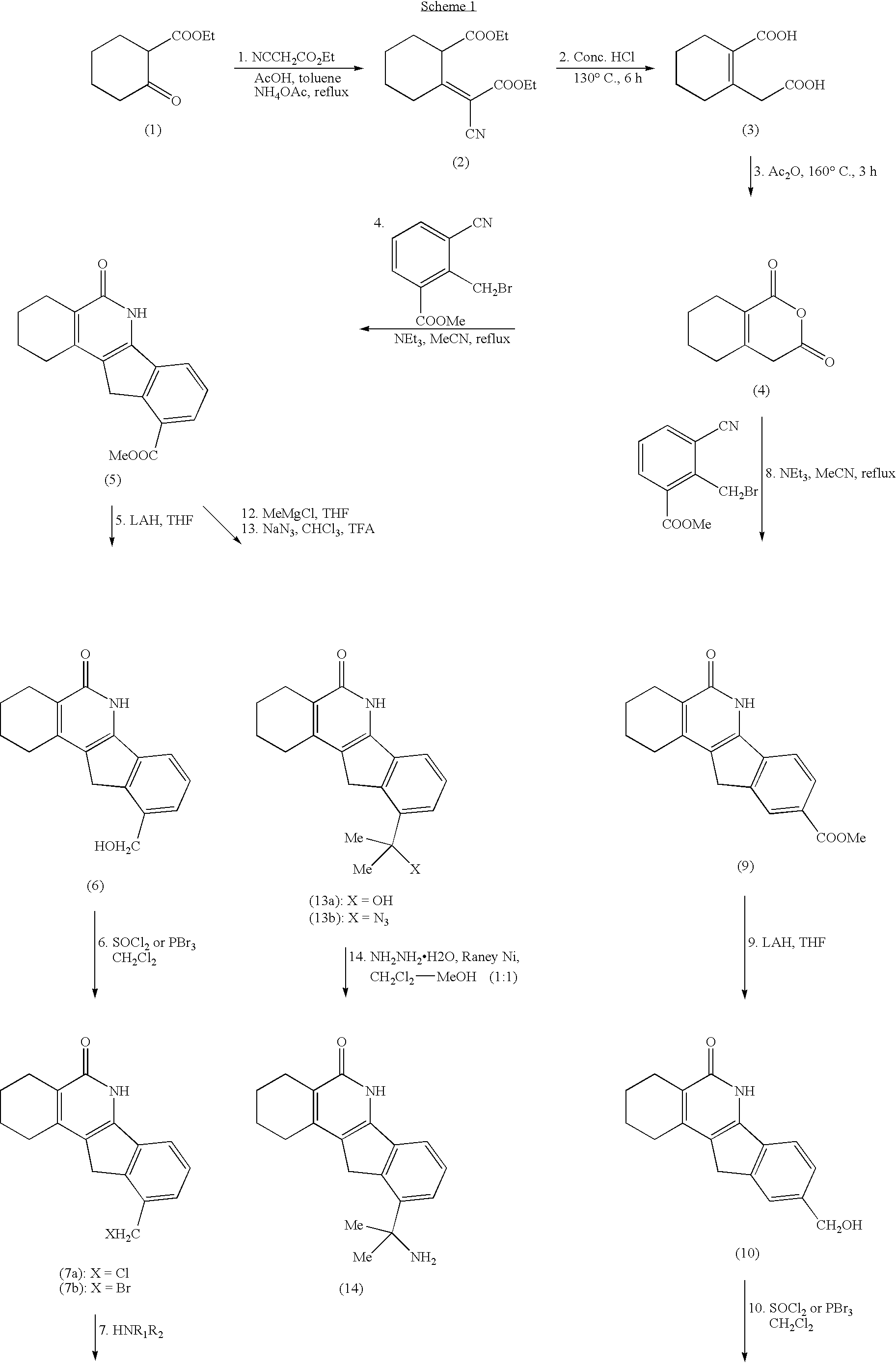

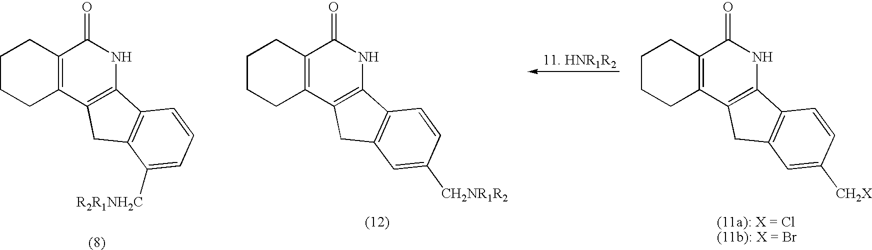

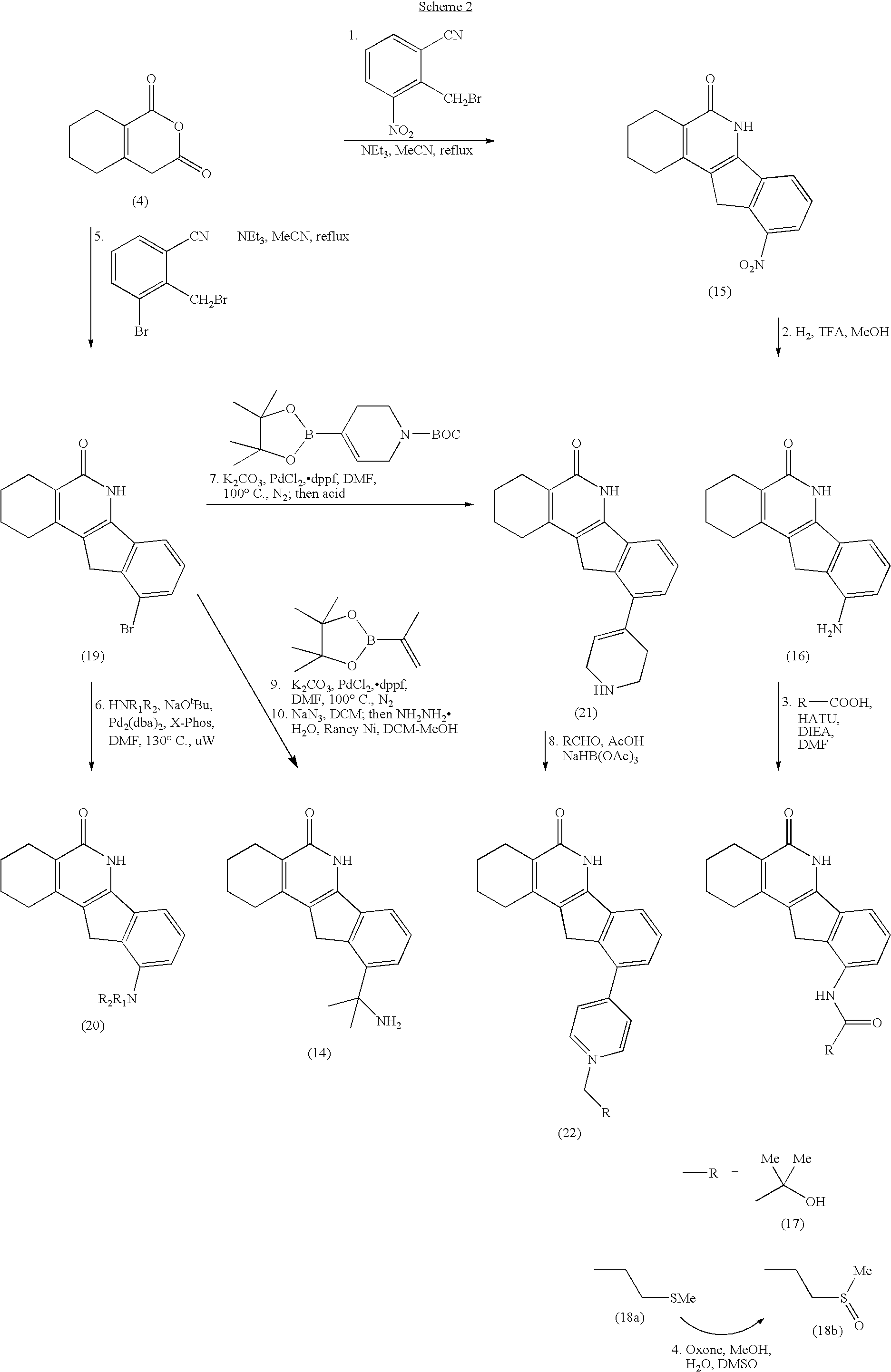

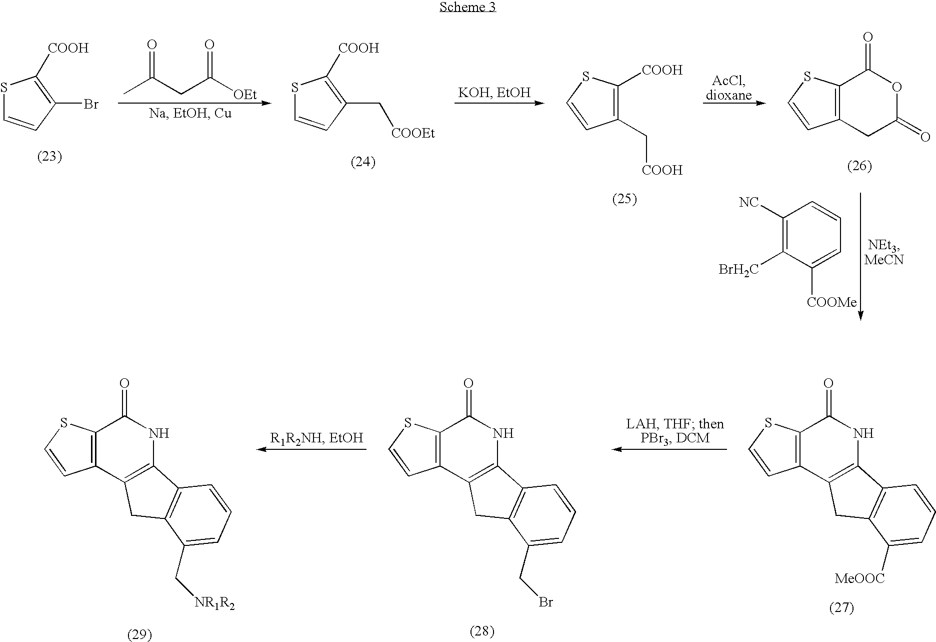

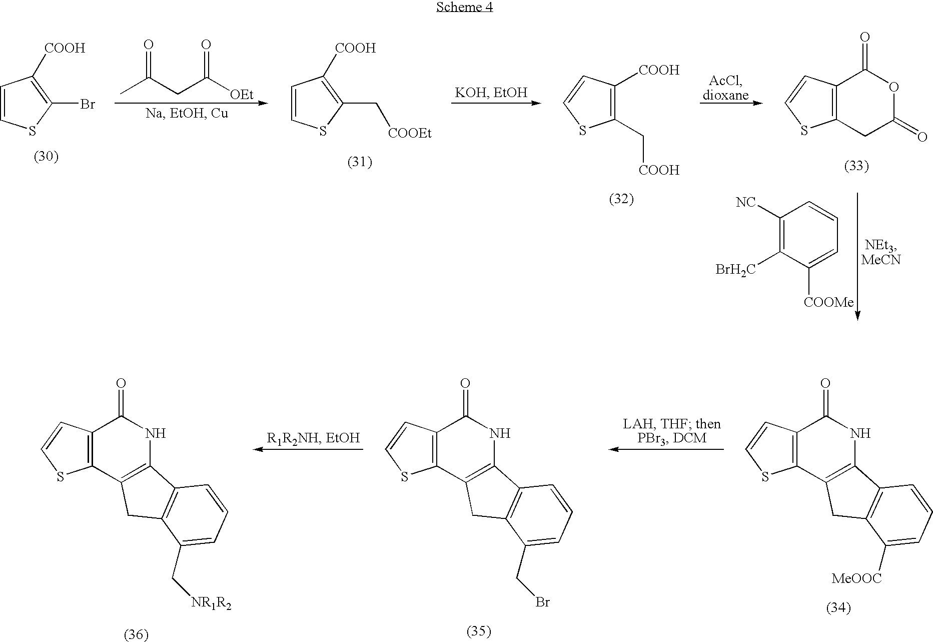

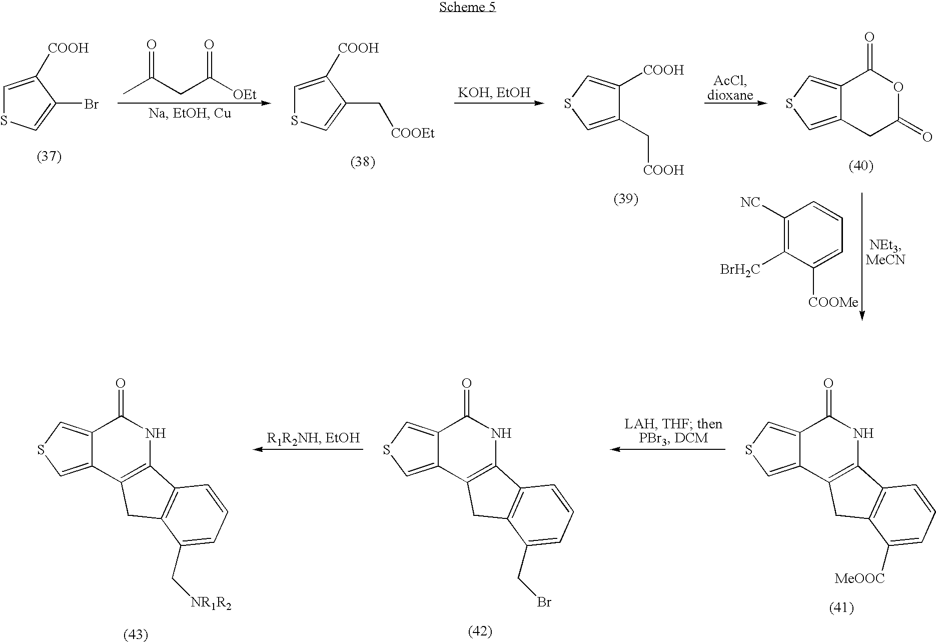

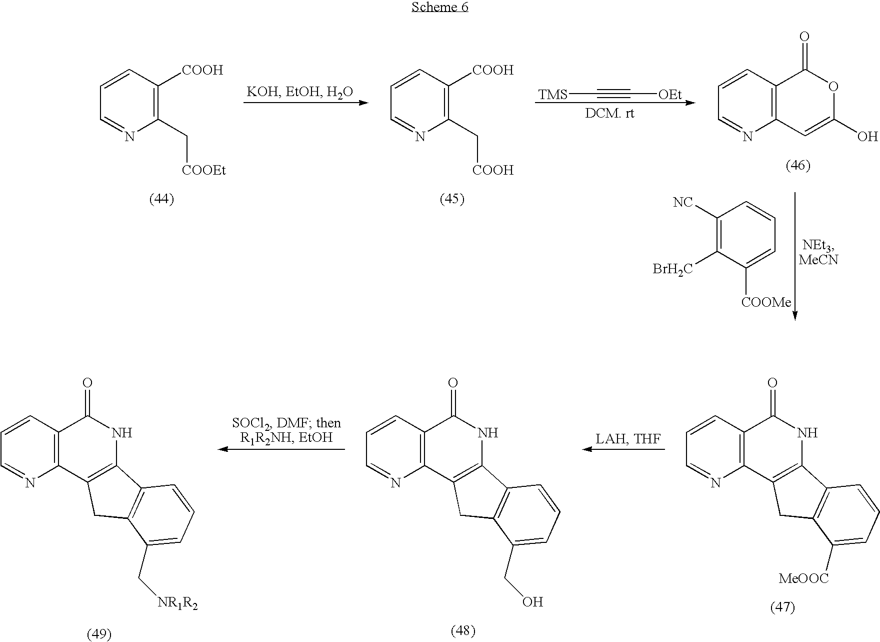

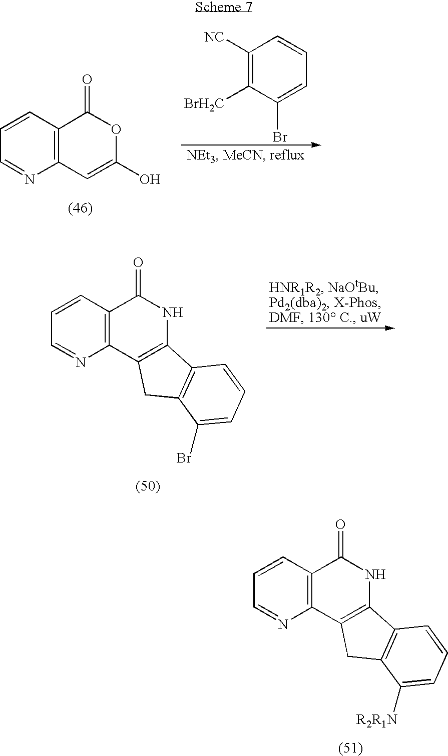

### 5.2 Experimental

With reference to Scheme 1:

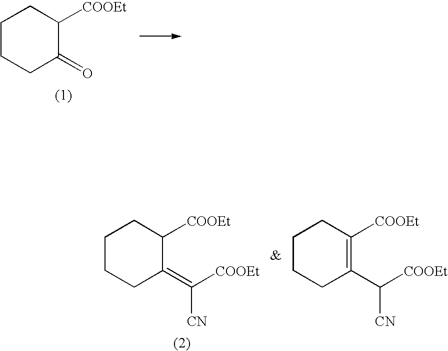

(E/Z)-Ethyl 2-(1-cyano-2-ethoxy-2-oxoethylidene)cyclohexanecarboxylate (2): A mixture of 2-carboethoxy-cyclohexanone (1) (20 gm, 117.6 mmol), cyanoethyl acetate (14.5 gm, 128.7 mmol), ammonium acetate (1.8 gm, 23.5 mmol), glacial acetic acid (5.3 ml, 94 mmol) and toluene (25 ml) was refluxed for 7 hr. It was cooled to room temperature and diluted with ether. The reaction mixture was then transferred into a separatory funnel and washed with water. The organic layer was collected and dried on sodium sulphate. It was concentrated and dried under vacuum to produce brown colored oil (30 gm, 97%).

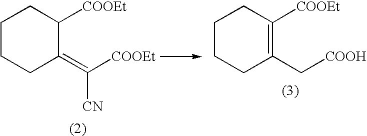

2-(Carboxymethyl)cyclohex-1-enecarboxylic acid (3): Diester (2)(58 g, 0.218 mol) was refluxed & stirred at 130° C. with concentrated HCl (200 ml). After 6 hr, additional HCl (50 ml) was added and the reaction mixture was refluxed for another 14 hr. Then it was cooled to 5° C. and the solid separated out was filtered. It was washed with water (3×100 ml), hexane (2×100 ml) and died in a vacuum oven at 50° C. to give diacid compound (3) (39.6 gm).

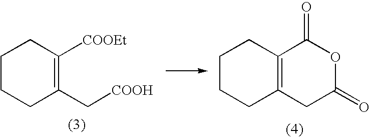

5,6,7,8-Tetrahydro-1H-isochromene-1,3(4H)-dione (4): Diacid (3)(15 gm) was suspended in acetic anhydride (30 ml) and the reaction mixture was stirred at 160° C. for 3 hr. The mixture was cooled to room temperature and then concentrated on rotavaporator and then under vacuum. The residue obtained was used as such for the next reaction (14.2 gm).

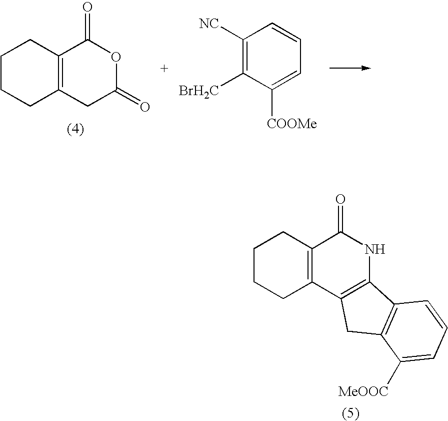

Methyl 5-oxo-2,3,4,5,6,11-hexahydro-1H-indeno[1,2-c]isoquinoline-10-carboxylate (5): To a mixture of anhydride (4) (6 gm) and bromomethyl-benzonitrile (3.5 gm) in acetonitrile (20 ml) was added triethylamine (4 ml) at room temperature. The reaction mixture was refluxed for 6 hr. It was cooled to room temperature. The solid separated out was filtered and washed with water (2×10 ml) and ethyl acetate (2×10 ml) and dried under vacuum at 50° C. to provide tetracyclic ester (5) (3.1 gm).

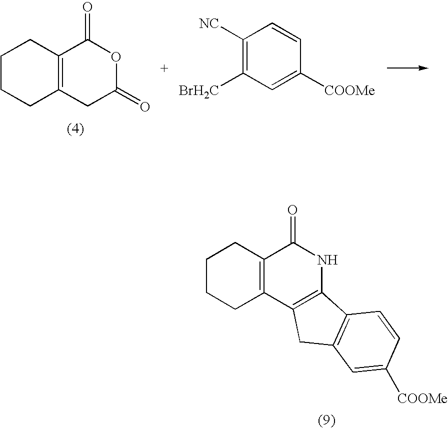

Methyl 5-oxo-2,3,4,5,6,11-hexahydro-1H-indeno[1,2-c]isoquinoline-9-carboxylate (9): Following the above reaction 9-carbomethoxy tetracycle (9) was prepared from hexahydro-homophthalic anhydride (4) and methyl 3-bromethyl-4-cyanobenzoate.

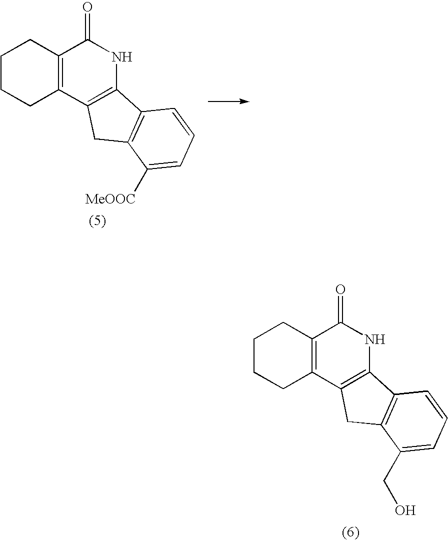

10-(Hydroxymethyl)-3,4,6,11-tetrahydro-1H-indeno[1,2-c]isoquinolin-5(2H)-one (6): To a suspension of ester (5) (1.96 gm) in THF (30 ml) was added slowly a solution of LAH in THF (2 M, 10 eq.). The reaction mixture was stirred at room temperature for 3 hr. LAH was quenched with slow addition of ethyl acetate (3 ml). The reaction mixture was stirred at room temperature for 0.5 hr, and poured into a solution of HCl (5M). It was stirred for 1 hr. Solid was collected by filtration and washed with water (2×5 ml), ethyl acetate (5 ml) and dried under vacuum to provide alcohol (6) (1.70 gm).

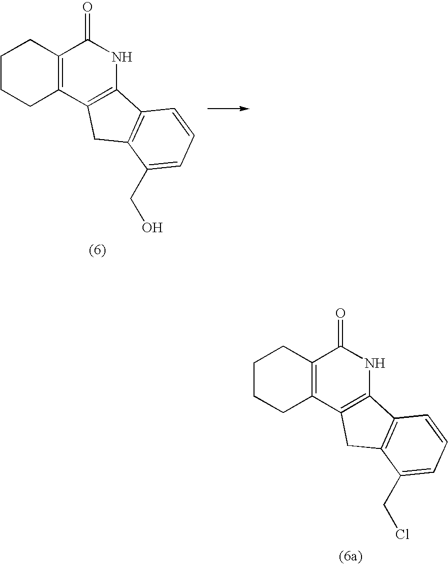

10-(Chloromethyl)-3,4,6,11-tetrahydro-M-indeno[1,2-c]isoquinolin-5(2H)-one (7a): To a mixture of alcohol (6) (1.5 gm) in dichloromethane (20 ml) was added dropwise thionyl chloride (4 ml). The mixture was stirred at room temperature for 1 hr, and then refluxed for 2 hr. The reaction mixture was cooled to 0° C. and methanol was added slowly, and stirred for additional 10 min. The mixture was poured in to the ice cold water (50 ml) and neutralized with sodium bicarbonate solid. The solid separated out was filtered and washed with water (2×10 ml), ethyl acetate (2×5 ml) and dried in a vacuum oven at 50° C. overnight to give chloro compound (7a) (1.6 gm).

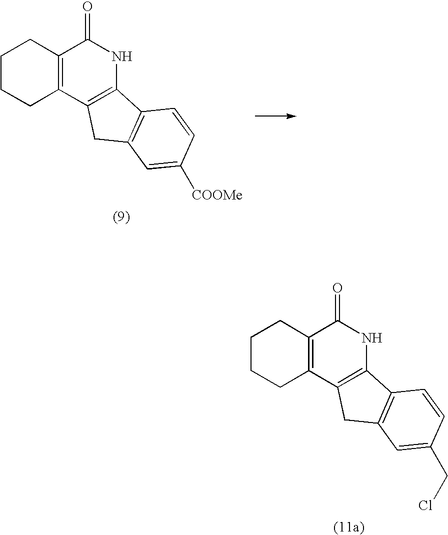

9-(Chloromethyl)-3,4,6,11-tetrahydro-1H-indeno[1,2-c]isoquinolin-5(2H)-one (11a): Following the above two reactions 9-chloromethyl tetracycle (11a) was prepared from 9-ester (9) and LAH in THF.

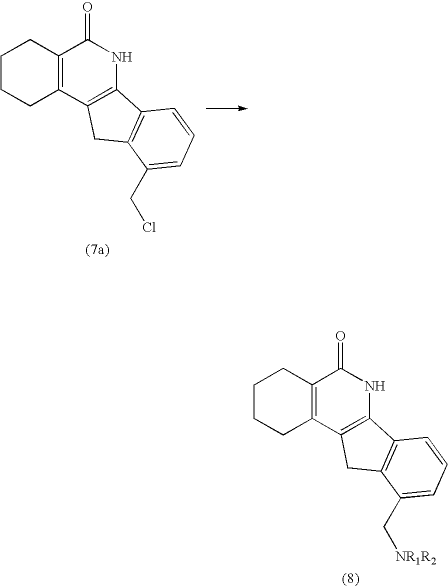

General procedure for substituted 10-(aminomethyl)-3,4,6,11-tetrahydro-1H-indeno[1,2-c]isoquinolin-5(2H)-one (8): A mixture of chloro compound (7a) and amine in DMF or ethanol or methanol or isopropyl alcohol was stirred at 60 to 100° C. until the reaction was complete. Triethyl amine was added when the hydrochloric salts of amines were used. The progress of reaction was monitored by tlc. The reaction mixture was cooled to room temperature and concentrated. The residue was treated with water (10 ml) and filtered. It was washed with water & ethyl acetate. The solid was dried in a vacuum oven at 50° C. for overnight.

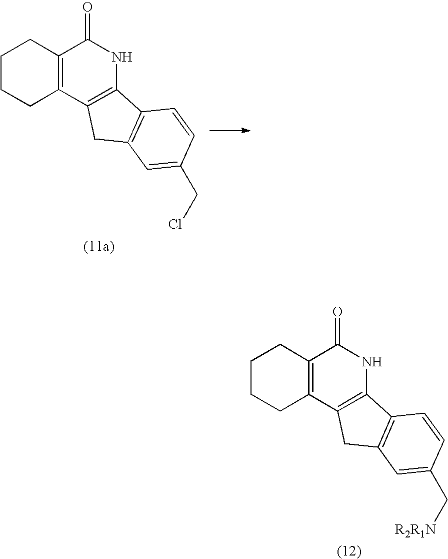

General procedure for substituted 9-(aminomethyl)-3,4,6,11-tetrahydro-1H-indeno[1,2-c]isoquinolin-5(2H)-one (12): A mixture of chloro compound (11a) and amine in DMF or ethanol or methanol or isopropyl alcohol was stirred at 60 to 100° C. until the reaction was complete. Triethyl amine was added when the hydrochloriride salts of amines were used. The progress of reaction was monitored by tlc. The reaction mixture was cooled to room temperature and concentrated. The residue was treated with water (10 ml) and filtered. It was washed with water & ethyl acetate. The solid was dried in a vacuum oven at 50° C. for overnight.

With reference to Scheme 2:

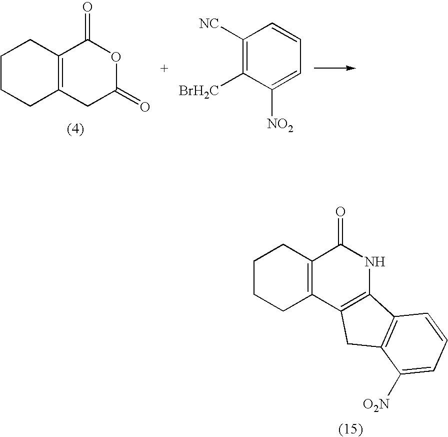

10-Nitro-3,4,6,11-tetrahydro-1H-indeno[1,2-c]isoquinolin-5(2H)-one (15): To a mixture of anhydride (4) (1.83 gm, 11 mmol) and bromomethyl-benzonitrile (2.41 gm, 10 mmol) in acetonitrile (40 ml) was added triethylamine (4.2 ml) at room temperature. The reaction mixture was refluxed for 16 hr. It was cooled to room temperature. The solid separated out was filtered and washed with water (2×10 ml) and ethyl acetate (2×10 ml) and dried under vacuum at 50° C. to provide the tetracyclic nitro compound (15) (2.63 gm, 93%).

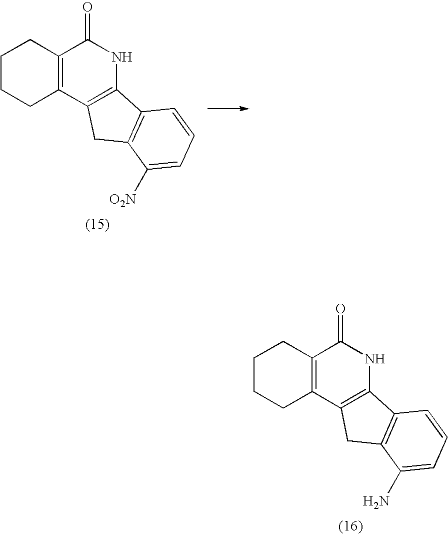

10-Amino-3,4,6,11-tetrahydro-1H-indeno[1,2-c]isoquinolin-5(2H)-one (16): A mixture of nitro compound (15) (565 mg, 2 mmol), palladium hydroxide (60 mg) in methanol and TFA was stirred under hydrogen atmosphere at room temperature for 7 hr. Reaction mixture was filtered and concentrated. The solid was dried under vacuum. The TFA salt (16) was then used as such for the next reaction.

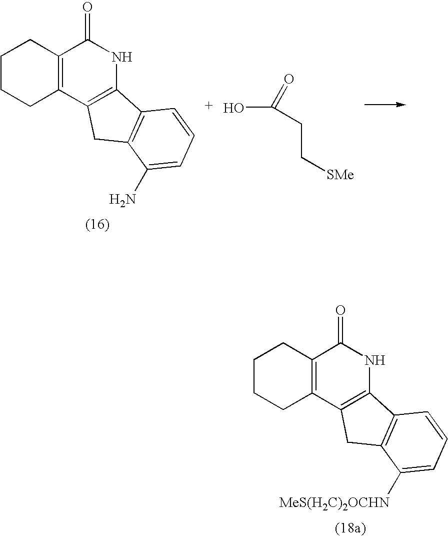

3-(Methylthio)-N-(5-oxo-2,3,4,5,6,11-hexahydro-1H-indeno[1,2-c]isoquinolin-10-yl)propanamide (18a): A mixture of TFA salt (16) (147 mg, 0.4 mmol), acid (53 mg, 0.44 mmol), HATU (167 mg, 0.44 mmol) and DIEA (0.3 ml, 1.6 mmol) in DMF (10 ml) was stirred at room temperature for 16 hr. The reaction mixture was then concentrated and solid was washed with methanol and water to give desired amide product (18a) (70 mg).

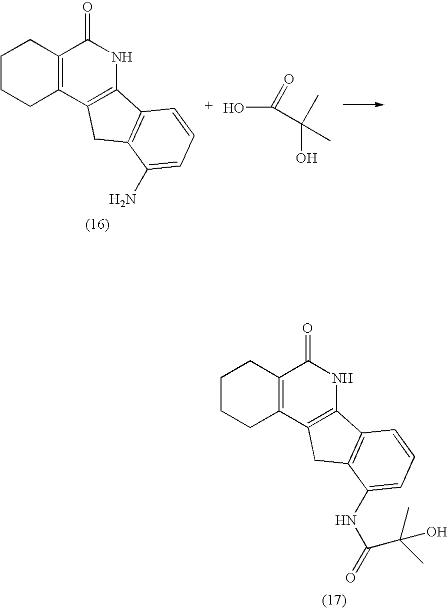

2-Hydroxy-2-methyl-N-(5-oxo-2,3,4,5,6,11-hexahydro-1H-indeno[1,2-c]isoquinolin-10-yl)propanamide (17): A mixture of TFA salt (16) (73 mg, 0.2 mmol), acid (23 mg, 0.22 mmol), HATU (83 mg, 0.22 mmol) and DIEA (0.14 ml, 0.8 mmol) in DMF (5 ml) was stirred at room temperature for 16 hr. The reaction mixture was then concentrated and purified by reverse phase column chromatography to produce desired product (17) (68 mg).

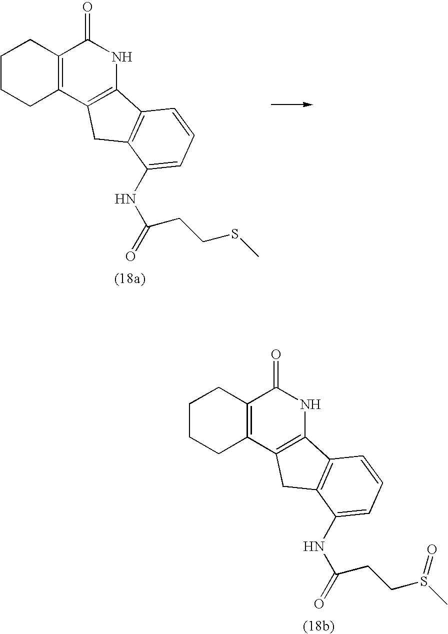

3-(Methylsulfinyl)-N-(5-oxo-2,3,4,5,6,11-hexahydro-1H-indeno[1,2-c]isoquinolin-10-yl)propanamide (18b): To a suspension of thiomethyl ether (18a) (60 mg, 0.17 mmol) in MOH/water/DMSO (1 ml), added a solution of Oxone (156 mg, 0.25 mmol) at room temperature and stirred for overnight. The starting material was separated from product using column chromatography. The fractions with product were concentrated to produce desired sulfoxide as the major product (18b).

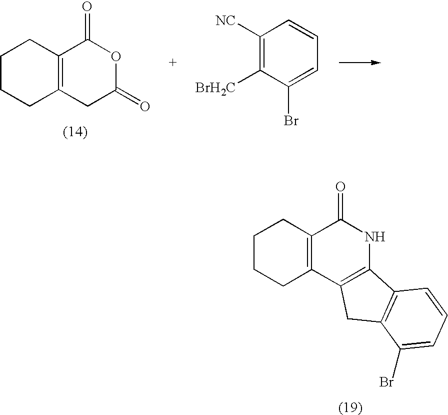

10-Bromo-3,4,6,11-tetrahydro-1H-indeno[1,2-c]isoquinolin-5(2H)-one (19): Following the above procedure, bromo tetracycle (19) was prepared from octahydro anhydride (14) and 3-bromo-2-bromomethyl-benzonitrile.

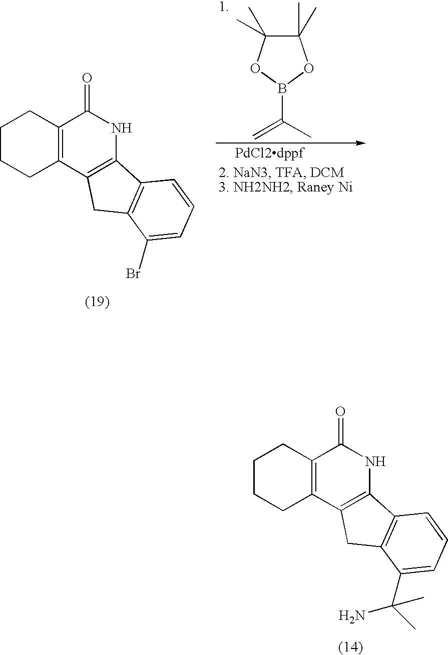

10-(2-Aminopropan-2-yl)-3,4,6,11-tetrahydro-1H-indeno[1,2-c]isoquinolin-5(2H)-one (14): To a suspension of bromo compound (19)(150 mg, 0.47 mmol) in DMF (5 ml) was added borolane (268 ul, 1.42 mmol), PdCl₂.dppf (23 mg, 0.028 mmol) and K₂CO₃ (196 mg, 1.42 mmol) at room temperature. The reaction mixture was stirred at 80° C. for overnight. It was concentrated and used for the next reaction. The crude mixture was suspended in TFA (0.7 ml) and DCM (4 ml) and treated with sodium azide. Reaction mixture was stirred at room temperature for 11 hr and after usual workup it was purified by column chromatography to produce gem dimethyl azido compound (71 mg). The azido compound was suspended in MeOH (4 ml) and DCM (4 ml), and treated with hydrazine hydrate (27 ul, 0.88 mmol) and catalytic Raney Ni. The reaction mixture was stirred at room temperature for 2 hr. It was filtered and filtrate was concentrated and purified by column chromatography to produce the desired product (14).

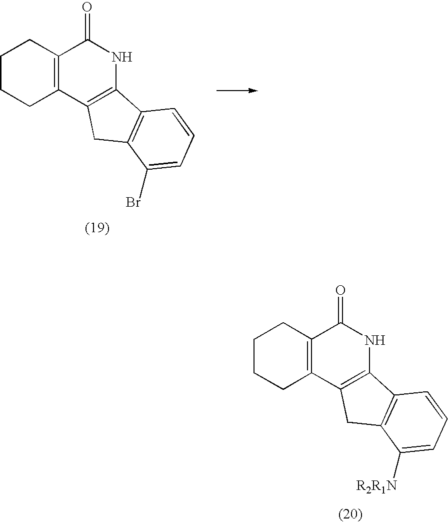

General procedure for 10-amino-3,4,6,11-tetrahydro-1H-indeno[1,2-c]isoquinolin-5(2H)-ones (20): A mixture of bromo compound (19) (126 mg, 0.4 mmol), amine (0.6 mmol), Pd₂(dba)₃ (7.4 mg, 0.008 mmol), S—X-Phos (9.6 mg, 0.02 mmol), sodium tert-butoxide (96 mg, 1 mmol) in DMF (4 ml) was degassed & then purged with nitrogen. The mixture was then irradiated under microwave at 130° C. for 20 min. It was concentrated and purified on the silica gel column using 0 to 5% methanol and DCM to provide final product (20).

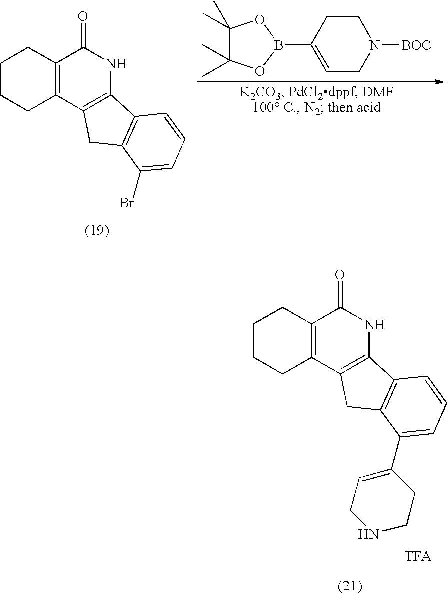

10-(1,2,3,6-Tetrahydropyridin-4-yl)-3,4,6,11-tetrahydro-1H-indeno[1,2-c]isoquinolin-5(2H)-one (21): A mixture of bromo compound (19) (316 mg, 1 mmol), dioxaboralane (325 mg, 1.05 mmol), potassium carbonate (415 mg, 3 mmol), PdCl₂.dppf (49 mg, 0.06 mmol) in DMF (8 ml) was heated under nitrogen at 100° C. for overnight. Reaction mixture was concentrated. After treatment with TFA, it produced desired TFA salt (21).

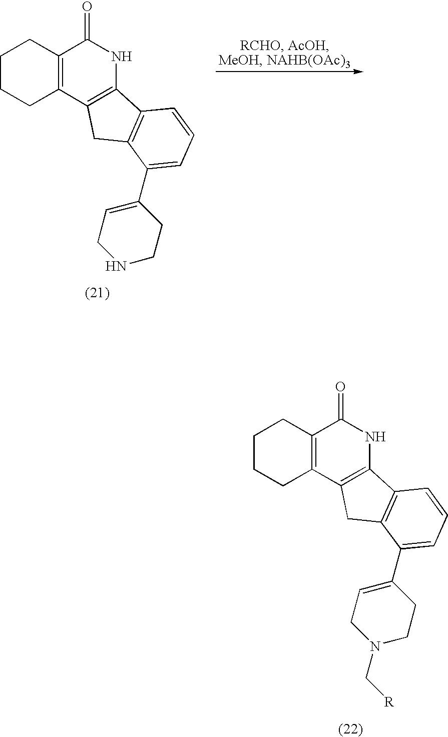

General procedure for 10-(1-alkyl-1,2,3,6-tetrahydropyridin-4-yl)-3,4,6,11-tetrahydro-1H-indeno[1,2-c]isoquinolin-5(2H)-one (22): A mixture of TFA salt (21) (70 mg, 0.16 mmol), aldehyde (11 ul, 0.14 mmol), NaHB(OAc)₃ (52 mg, 0.24 mmol), AcOH (100 ul) in methanol-DCM (1:1, 3 ml) was stirred at room temperature for 16 hr. Additional aldehyde and NaHB(OAc)₃ was added (1 eq. each). It was diluted with saturated sodium bicarbonate and extracted with DCM. The organic layer was dried on sodium sulphate and concentrated. The residue obtained was purified by reverse phase HPLC to provide desired product (22).

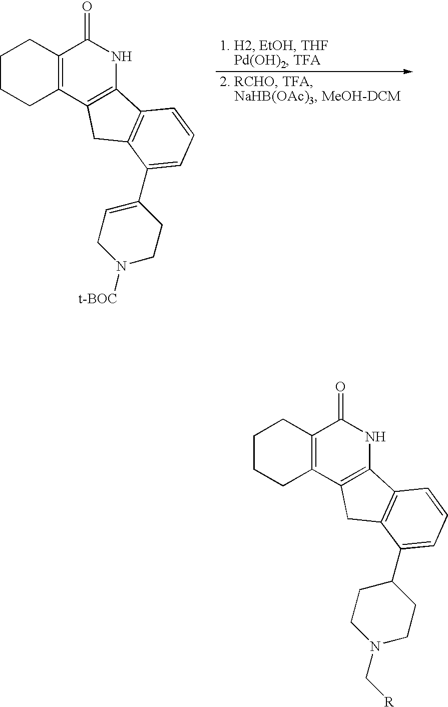

General procedure for 10-(1-alkylpiperidin-4-yl)-3,4,6,11-tetrahydro-1H-indeno[1,2-c]isoquinolin-5(2H)-one: A suspension of 1-tBoc-1,2,3,6-tetrahydropyridine compound, Pd(OH)₂ (140 mg) in EtOH (200 ml), THF (50 ml) and TFA (1 ml) was stirred under hydrogen atmosphere using hydrogen gas balloon. Reaction mixture was filtered after 17 hr and concentrated. As described above, the TFA salt was then alkylated to give desired product.

With reference to Schemes 3, 4 and 5:

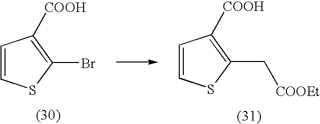

2-(2-Ethoxy-2-oxoethyl)thiophene-3-carboxylic acid (31): Sodium (1.31 gm, 57 mmol) was placed in a oven dried flask & anhydrous ethanol (75 ml) was added slowly to keep the temperature between 40-50° C. After 1 hr, sodium was completely dissolved. Ethyl acetoacetate (4.55 ml, 36 mmol) was added to the cold solution of sodium ethoxide. The reaction mixture was stirred for 5 min and then treated with 2-bromo-thiophene 3-carboxylic acid (30) (5 gm, 24 mmol) and copper (247 mg, 3.9 mmol). The combined reaction mixture was refluxed for 13 hr. It was poured on water and the suspension was filtered. The pH was adjusted from 11.0 to 3 using concentrated HCl. Solid was filtered, washed with water and dried to give desired product (31) (5.1 gm, 99%).

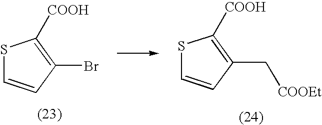

3-(2-Ethoxy-2-oxoethyl)thiophene-2-carboxylic acid (24): Similarly 3-(2-ethoxy-2-oxoethyl)thiophene-2-carboxylic acid (24) was prepared from 3-bromo-thiophene 2-carboxylic acid (23), sodium, ethyl acetoacetate, copper and sodium ethoxide.

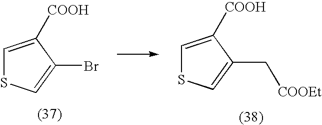

4-(2-Ethoxy-2-oxoethyl)thiophene-3-carboxylic acid (38): Similarly 4-(2-ethoxy-2-oxoethyl)thiophene-3-carboxylic acid (38) was prepared from 4-bromo-thiophene 3-carboxylic acid (37) (5 gm, 24 mmol), sodium (1.31 gm, 57 mmol), ethyl acetoacetate (4.55 ml, 36 mmol), copper (247 mg, 3.9 mmol) and sodium ethoxide.

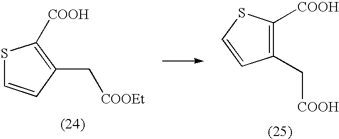

3-(Carboxymethyl)thiophene-2-carboxylic acid (25): To a suspension of ester (24) (2.57 gm, 12 mmol) in water (10 ml) was added KOH (2.7 gm) at 0° C. After the reaction mixture became homogeneous, the cold bath was removed and the reaction was stirred at room temperature for 16 hr. Reaction mixture was then acidified with HCl (12 M aqueous solution) at 0° C. The solid precipitated out was filtered, washed with water and dried under vacuum to give diacid (25) (2.23 gm, 49%).

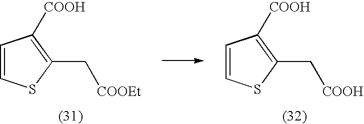

2-(Carboxymethyl)thiophene-3-carboxylic acid (32): To a suspension of ester (31) (5.1 gm, 23.8 mmol) in water (20 ml) was added KOH (4.9 gm) at 0° C. After the reaction mixture became homogeneous, the cold bath was removed and the reaction was stirred at rt for 16 hr. Reaction mixture was then acidified with HCl (12 M aqueous solution) at 0° C. The solid precipitated out was filtered, washed with water and dried under vacuum to give diacid (32) (3.88 gm, 87%).

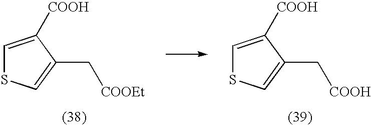

4-(Carboxymethyl)thiophene-3-carboxylic acid (39): To a suspension of ester (38) (5.17 gm, 24 mmol) in water (20 ml) was added KOH (4.9 gm) at 0° C. After the reaction mixture became homogeneous, the cold bath was removed and the reaction was stirred at rt for 16 hr. Reaction mixture was then acidified with HCl (12 M aqueous solution) at 0° C. The solid precipitated out was filtered, washed with water and dried under vacuum to give diacid (39) (3.23 gm, 72%).

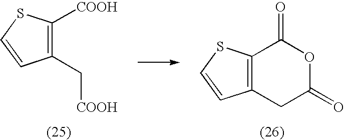

4H-Thieno[2,3-c]pyran-5,7-dione (26): A mixture of diacid (25) (1 gm, 5.4 mmol) and acetyl chloride (15 ml) in dioxane (100 ml) was refluxed for 2 hr. The reaction mixture was concentrated. The solid obtained was collected by filtration and was directly used for the next reaction.

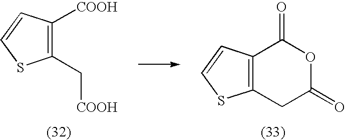

4H-Thieno[3,2-c]pyran-4,6(7H)-dione (33): A mixture of diacid (32)(3 gm, 16.1 mmol) and acetyl chloride (30 ml) in dioxane (200 ml) was refluxed for 3 hr at 130° C. The reaction mixture was concentrated and the solid obtained was collected by filtration (2.7 gm, 100%).

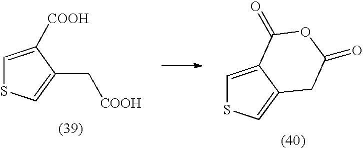

4H-Thieno[3,4-c]pyran-4,6(7H)-dione (40): A mixture of diacid (39) (3 gm, 16.1 mmol) and acetyl chloride (30 ml) in dioxane (200 ml) was refluxed for 3 hr at 130° C. The reaction mixture was concentrated and the solid obtained was collected by filtration (2.79 gm, 100%).

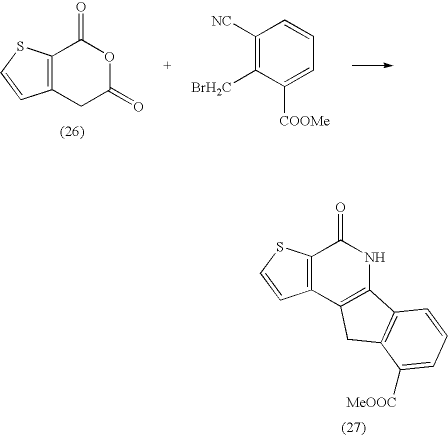

Methyl 4-oxo-5,10-dihydro-4H-indeno[1,2-b]thieno[3,2-d]pyridine-9-carboxylate (27): To a mixture of anhydride (26) (0.9 gm, 5.4 mmol) and bromomethyl-benzonitrile (1.37 gm, 5.1 mmol) in acetonitrile (10 ml) was added triethylamine (2.25 ml) at room temperature. The reaction mixture was refluxed for 16 hr. It was cooled to room temperature. The solid separated out was filtered and washed with water (2×10 ml) and ethyl acetate (2×10 ml) and dried under vacuum at 50° C. to provide tetracyclic ester (27) (1.3 gm, 82%).

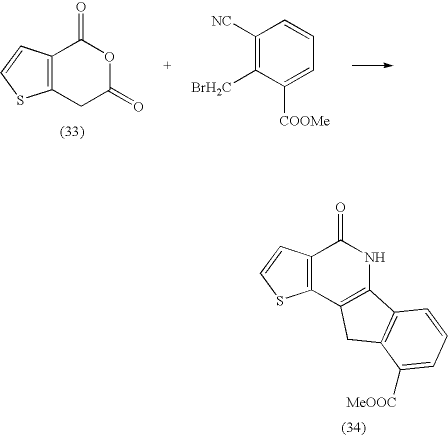

Methyl 4-oxo-5,10-dihydro-4H-indeno[1,2-b]thieno[2,3-d]pyridine-9-carboxylate (34): To a mixture of anhydride (33) (1 gm, 5.94 mmol) and bromomethyl-benzonitrile (1.51 gm, 5.94 mmol) in acetonitrile (10 ml) was added triethylamine (2.5 ml) at −50° C. The reaction mixture was stirred at room temperature for 1 hr and refluxed at 85° C. for 16 hr. It was cooled to room temperature. The solid separated out was filtered and washed with water (2×10 ml) and ethyl acetate (2×10 ml) and dried under vacuum at 50° C. to provide tetracyclic ester (34) (1.35 gm, 76%).

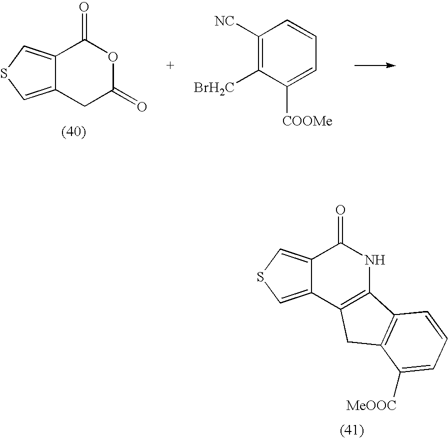

Methyl 4-oxo-5,10-dihydro-4H-indeno[1,2-b]thieno[3,4-d]pyridine-9-carboxylate (41): To a mixture of anhydride (40) (1 gm, 5.94 mmol) and bromomethyl-benzonitrile (1.51 gm, 5.94 mmol) in acetonitrile (10 ml) was added triethylamine (2.5 ml) at −50° C. The reaction mixture was stirred at room temperature for 1 hr and refluxed at 85° C. for 16 hr. It was cooled to room temperature. The solid separated out was filtered and washed with water (2×10 ml) and ethyl acetate (2×10 ml) and dried under vacuum at 50° C. to provide tetracyclic ester (41) (806 mg, 45%)

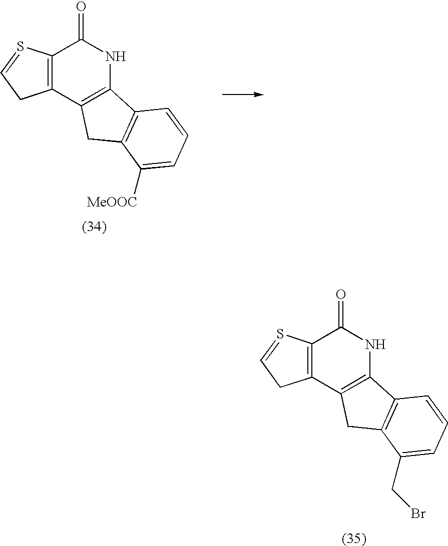

9-(Bromomethyl)-5,10-dihydro-4H-indeno[1,2-b]thieno[3,2-d]pyridin-4-one (28): To a suspension of ester (27) (1 gm, 3.36 mmol) in THF (20 ml) was added slowly a solution of LAH in THF (2 M, 5 ml, 3 eq.). The reaction mixture was stirred at room temperature for 5 hr. The LAH was quenched with slow addition of ethyl acetate (6.6 ml). The reaction mixture was then stirred at room temperature for 0.5 hr, and poured into a solution of HCl (5M). It was stirred for 1 hr. Solid was collected by filtration and washed with dil HCl, water (2×5 ml), and DCM (5 ml), and dried under vacuum to provide alcohol (870 mg). To a suspension of alcohol (800 mg, 2.97 mmol) in THF (16 ml) was added PBr₃ in DCM (1 M, 8.9 ml, 8.91 mmol), and stirred at room temperature for 2 days. It was quenched with water (10 ml) and solid was collected by filtration. It was dried to produce bromo compound (28) (775 mg, 78%).

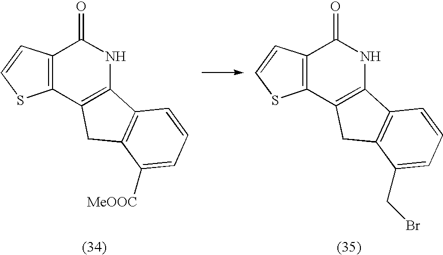

9-(Bromomethyl)-5,10-dihydro-4H-indeno[1,2-b]thieno[2,3-d]pyridin-4-one (35): To a suspension of ester (34) (1 gm, 3.36 mmol) in THF (20 ml) was added slowly a solution of LAH in THF (2 M, 5 ml, 3 eq.). The reaction mixture was stirred at room temperature for 7 hr. The LAH was quenched with slow addition of ethyl acetate (6.6). The reaction mixture was then stirred at room temperature for 0.5 hr, and poured into a solution of HCl (5M). It was stirred for 0.5 hr. Solid was collected by filtration and washed with dil HCl, water (2×5 ml), and DCM (5 ml), and dried under vacuum to provide alcohol (813 mg). To a suspension of alcohol (800 mg, 2.97 mmol) in THF (16 ml) was added PBr₃ in DCM (1 M, 8.9 ml, 8.91 mmol), and stirred at room temperature for 2 days. It was quenched with water (10 ml) and solid was collected by filtration. It was dried to produce bromo compound (35) (815 mg, 83%).

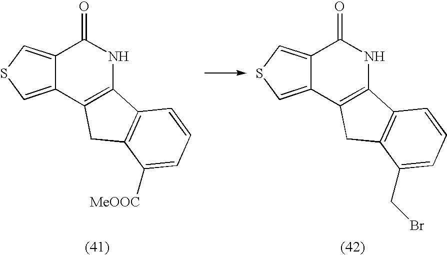

9-(Bromomethyl)-5,10-dihydro-4H-indeno[1,2-b]thieno[3,4-d]pyridin-4-one (42): To a suspension of ester (41) (0.776 gm, 3.36 mmol) in THF (16 ml) was added slowly a solution of LAH in THF (2 M, 4 ml, 3 eq.). The reaction mixture was stirred at 0° C. for 6 hr. The LAH was quenched with slow addition of ethyl acetate (5.3 ml). The reaction mixture was then stirred at room temperature for 0.5 hr, and poured into a solution of HCl (5M). It was stirred for 0.5 hr. Solid was collected by filtration and washed with dil HCl, water (2×5 ml), and DCM (5 ml), and dried under vacuum to provide alcohol. To a suspension of crude alcohol in THF (16 ml) was added PBr₃ in DCM (1 M, 7.83 ml, 7.83 mmol), and bromo compound (42) was prepared as described above.

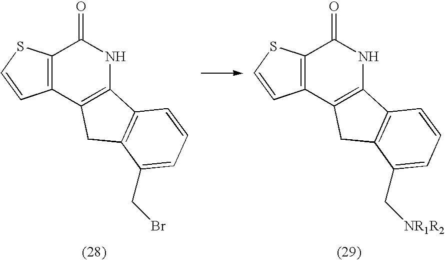

General procedure for 9-(aminomethyl)-5,10-dihydro-4H-indeno[1,2-b]thieno[3,2-d]pyridin-4-one (29): A mixture of bromo compound (28) (100 mg, 0.3 mmol), amine (excess) in ethanol (2 ml) was refluxed for 16 hr. The solvent was evaporated and the mixture was suspended in DMF (4 ml), DMSO (0.5 ml) and TFA (2 ml) and filtered. The filtrate was purified by silica gel column and followed by reverse phase chromatography.

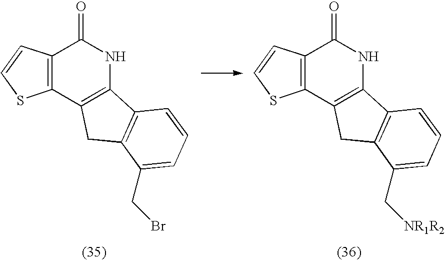

General procedure for 9-(aminomethyl)-5,10-dihydro-4H-indeno[1,2-b]thieno[2,3-d]pyridin-4-one (36): A mixture of bromo compound (35) (100 mg, 0.3 mmol), amine (excess) in ethanol (2 ml) was refluxed for 16 hr. The solvent was evaporated and the mixture was suspended in DMF (4 ml), DMSO (0.5 ml) and TFA (2 ml) and filtered. The filtrate was purified by silica gel column and followed by reverse phase chromatography.

With reference to Schemes 6 and 7:

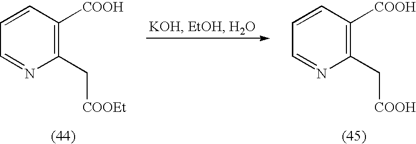

2-(Carboxymethyl)nicotinic acid (45): To a solution of ester (44) (2.09 gm, 10 mmol) in ethanol (50 ml) was added a cold solution of KOH (16.83 gm) in water (20 ml) and ethanol (50 ml) at 0° C. The reaction mixture was stirred while warming up to room temperature. After 1 hr, it was concentrated to ⅓ of its original volume and quenched with 10% aqueous HCl. The residue was extracted with ethyl acetate, washed with water and dried on sodium sulphate. It was concentrated under vacuum to give desired product (45) (1.81 gm, 49%). Above reaction was also carried out in isopropyl alcohol and LiOH at room temperature.

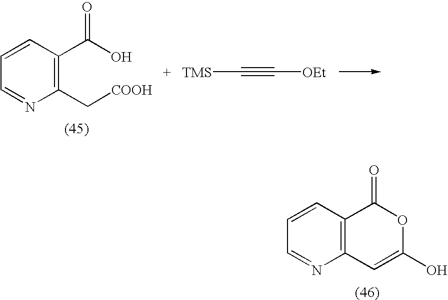

7-Hydroxy-5H-pyrano[4,3-b]pyridin-5-one (46): A mixture of anhydride (45) (862 mg, 4.76 mmol) and (ethoxyethynyl)trimethylsilane (2.03 gm, 14.28 mmol) in DCM (15 ml) was stirred at room temperature for 3 days. The reaction mixture was concentrated and suspended in toluene and hexane (1:1) and the solid was isolated by filtration. It was washed with hexane, dried under vacuum to provide anhydride (46) (630 mg, 81%).

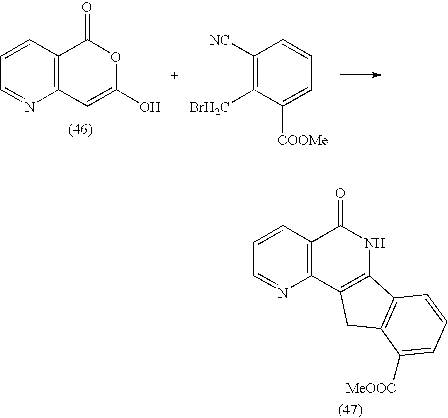

Methyl 5-oxo-6,11-dihydro-5,1-indeno[1,2-h][1,6]naphthyridine-10-carboxylate (47): A solution of anhydride (46) (630 mg, 3.86 mmol) and bromo compound (981 mg, 3.86 mmol) in acetonitrile (20 ml) was treated with triethyl amine (1.61 ml, 11.59 mmol) at −50° C. The reaction mixture was refluxed for 18 hr. Reaction mixture was cooled to room temperature and solid separated out was filtered, washed with MeCN, water and MeCN. It was dried under vacuum to provide ester (47) (507 mg, 46%).

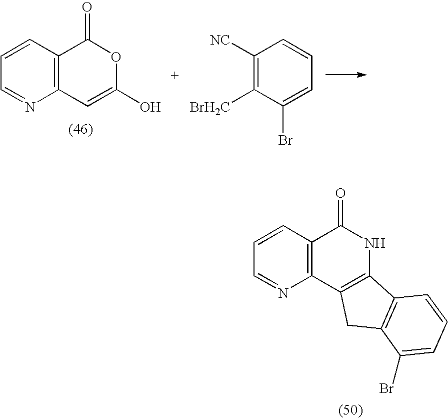

10-Bromo-6,11-dihydro-5H-indeno[1,2-h][1,6]naphthyridin-5-one (50): Following the above reaction, bromo tetracycle(50) was made from anhydride (46) and corresponding bromo compound.

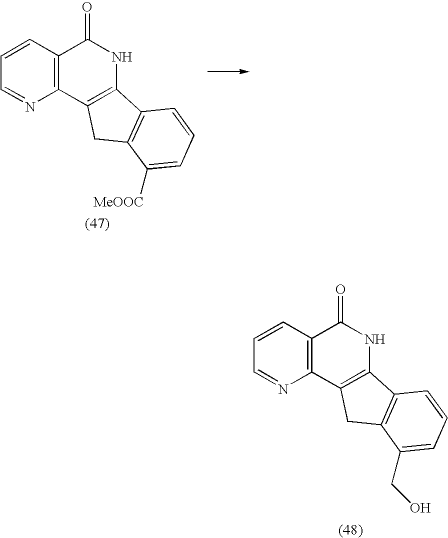

10-(Hydroxymethyl)-6,11-dihydro-5H-indeno[1,2-h][1,6]naphthyridin-5-one (48): To a suspension of ester (47) (507 mg, 1.73 mmol) in THF (20 ml) was added dropwise a solution of LAH in THF (2M, 2.37 ml, 4.67 mmol) at room temperature. The resulting mixture was stirred at room temperature for 6 hr and then quenched with dilute HCl (1N, 12 ml). The orange colored solid was collected by filtration, washed with water and ethyl acetate. It was dried under vacuum to yield desired product (48).

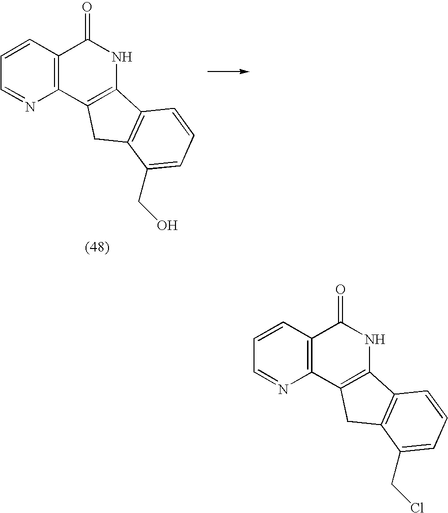

10-(Chloromethyl)-6,11-dihydro-5H-indeno[1,2-h][1,6]naphthyridin-5-one: A mixture of alcohol (46 mg, 0.17 mmol), thionyl chloride (123 ul, 1.7 mmol) in DCM (10 ml) was stirred at room temperature. Reaction mixture was stirred for 30 min and concentrated. The residue was used as such for the next reaction.

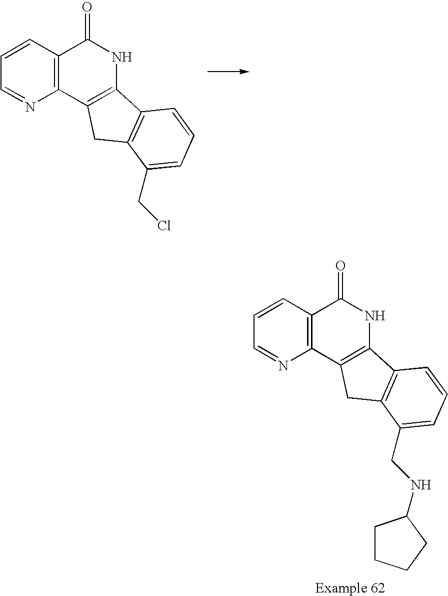

10-(Cyclopentylamino)-6,11-dihydro-5H-indeno[1,2-h][1,6]naphthyridin-5-one (Example 62): The above chloro compound was suspended in isopropyl alcohol, triethyl amine (1 ml) and cyclopentylamine (1 ml, excess) and refluxed at 100° C. for 1 hr. The reaction mixture was cooled to room temperature and concentrated. The residue obtained was purified on reverse phase column to provide desired product (Example 62).

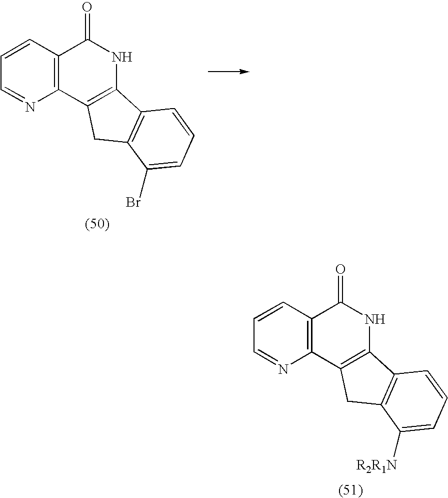

General procedure for 10-(amino)-6,11-dihydro-5H-indeno[1,2-h][1,6]naphthyridin-5-one (51): A mixture of 10-bromo compound (50) (94 mg, 0.3 mmol), amine (64 mg, 0.45 mmol), Pd₂(dba)₃ (5.5 mg, 0.006 mmol), S—X-Phos (7.2 mg, 0.015 mmol), sodium tert-butoxide (72 mg, 0.75 mmol) in DMF (4 ml) was degassed and then purged with nitrogen. The mixture was irradiated under microwave at 130° C. for 20 min. It was concentrated and purified on the silica gel column using 0 to 7% methanol and DCM. The residue obtained was further purified on the reverse phase column to provide final product (51).

### 5.3 EXAMPLES

The following examples were prepared according to the methods, schemes and experimental described above.

### Example 1

### 10-((4-hydroxypiperidin-1-yl)methyl)-3,4,6,11-tetrahydro-1H-indeno[1,2-c]isoquinolin-5(2H)-one

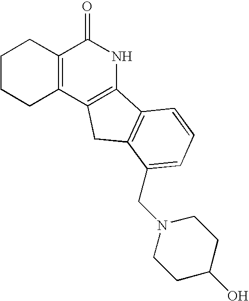

¹H NMR (DMSO-d6): d 12.09 (bs, 1H), 7.85 (d, J=7.8 Hz, 1H), 7.32 (t, J=7.5 Hz, 1H); 7.25 (d, J=7.2 Hz, 1H); 4.53 (d, J=3.6 Hz, 1H); 3.57 (s, 2H), 3.52 (s, 2H); 3.44 (m, 1H); 2.63 (m, 2H), 2.40 (m, 2H), 2.05 (m, 2H), 1.71 (m, 4H), 1.38 (m, 2H).

### Example 2

### 10-(piperidin-1-ylmethyl)-3,4,6,11-tetrahydro-1H-indeno[1,2-c]isoquinolin-5(2H)-one

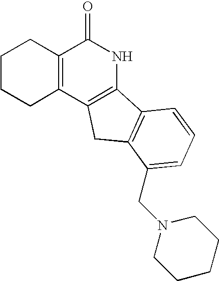

¹H NMR (DMSO-d6): d 12.29 (bs, 1H); 7.84 (d, J=7.2 Hz, 1H), 7.30 (m, 2H); 3.58 (s, 2H), 3.53 (s, 2H); 2.64 (m, 2H); 2.40 (m, 2H); 2.35 (m, 4H); 1.71 (m, 4H); 1.48 (m, 4H); 1.39 (m, 4H).

### Example 3

### 10-((4-hydroxypiperidin-1-yl)methyl)-3,4,6,11-tetrahydro-1H-indeno[1,2-c]isoquinolin-5(2H)-one hydrochloride

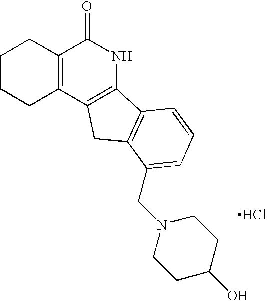

¹H NMR (DMSO-d6): d 12.18 (bs, 1H); 10.33 (bs, 1H); 8.03 (d, J=7.8 Hz, 1H); 7.64 (t, J=7.2 Hz, 1H); 7.47 (t, J=7.5 Hz, 1H), 4.38 (dd, J=4.5 Hz, 15.3 Hz, 2H)); 3.82 (m, 2H); 3.60 (m, 1H); 3.19 (m, 4H); 2.64 (s, 2H); 2.41 (s, 2H); 1.95 (m, 2H); 1.71 (m, 6H).

### Example 4

### 10-(piperidin-1-ylmethyl)-3,4,6,11-tetrahydro-1H-indeno[1,2-c]isoquinolin-5(2H)-one hydrochloride

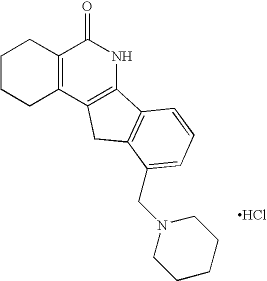

¹H NMR (DMSO-d6): d 12.22 (bs, 1H); 10.39 (bs, 1H); 8.02 (d, J=7.5 Hz, 1H), 7.63 (d, J=7.5 Hz, 1H); 7.25 (t, J=7.5 Hz, 1H); 4.34 (d, J=4.8 Hz, 2H); 3.82 (s, 2H); 3.30 (m, 2H); 2.98 (m, 2H); 2.65 (m, 2H); 2.41 (m, 2H), 1.71 (m, 9H); 1.38 (m, 1H).

### Example 5

### 10-(azepan-1-ylmethyl)-3,4,6,11-tetrahydro-1H-indeno[1,2-c]isoquinolin-5(2H)-one

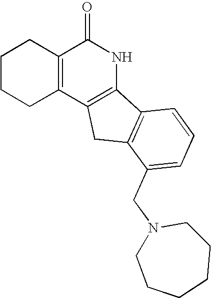

¹H NMR (DMSO-d6): d 12.29 (bs, 1H), 7.82 (m, 1H), 7.30 (m, 2H), 3.69 (s, 2H), 3.59 (s, 2H); 2.63 (m, 2H); 2.57 (m, 4H), 2.40 (m, 2H); 1.71 (m, 4H); 1.55 (m, 8H).

### Example 6

### 10-(pyrrolidin-1-ylmethyl)-3,4,6,11-tetrahydro-1H-indeno[1,2-c]isoquinolin-5(2H)-one

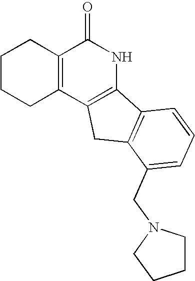

¹H NMR (DMSO-d6): d 12.11 (bs, 1H); 7.83 (d, J=6.9 Hz, 1H); 7.28 (m, 2H); 3.68 (s, 2H); 3.58 (s, 2H); 2.64 (m, 2H); 2.43 (m, 6H); 1.71 (m, 8H).

### Example 7

### 10-(azepan-1-ylmethyl)-3,4,6,11-tetrahydro-1H-indeno[1,2-c]isoquinolin-5(2H)-one hydrochloride

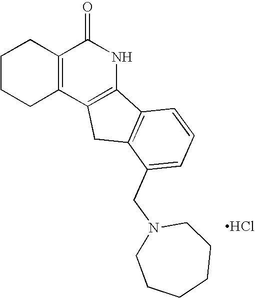

¹H NMR (DMSO-d6): d 11.04 (bs, 1H); 7.98 (d, J=7.8 Hz, 1H); 7.77 (d, J=7.8 Hz, 1H); 7.45 (t, J=7.8 Hz, 1H); 4.37 (d, J=4.8 Hz, 2H); 3.84 (s, 2H); 3.27 (m, 2H); 3.11 (m, 2H); 2.63 (m, 2H); 2.40 (m, 2H); 1.85 (m, 4H); 1.73 (m, 4H); 1.61 (m, 4H).

### Example 8

### 10-(pyrrolidin-1-ylmethyl)-3,4,6,11-tetrahydro-1H-indeno[1,2-c]isoquinolin-5(2H)-one hydrochloride

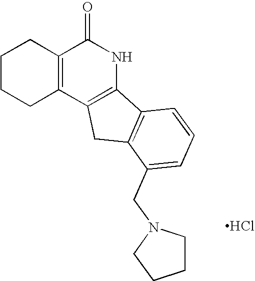

¹H NMR (DMSO-d6): d 11.26 (bs, 1H); 7.99 (d, J=7.5 Hz, 1H); 7.66 (d, J=7.5 Hz, 1H); 7.45 (t, J=7.5 Hz, 1H); 4.43 (d, J=5.4 Hz, 2H); 3.88 (s, 2H); 3.37 (m, 2H); 3.11 (m, 2H); 2.63 (m, 2H); 2.40 (m, 2H); 2.01 (m, 2H); 1.91 (m, 2H); 1.72 (m, 4H).

### Example 9

### 10-((4-ethoxypiperidin-1-yl)methyl)-3,4,6,11-tetrahydro-1H-indeno[1,2-c]isoquinolin-5(2H)-one

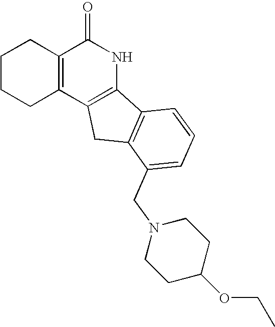

¹H NMR (DMSO-d6): d 12.25 (bs, 1H), 7.83 (d, (d, J=7.2 Hz, 1H); 7.33 (t, (d, J=6.9 Hz, 1H); 7.25 (d, J=6.9 Hz, 1H); 3.58 (s, 2H), 3.54 (s, 2H), 3.39 (q, J=6.9 Hz, 2H); 3.24 (m, 1H), 2.63 (m, 4H), 2.40 (m, 2H), 2.09 (m, 2H), 1.74 (m, 2H), 1.70 (m, 4H); 1.41 (m, 2H); 1.06 (t, J=6.9 Hz, 3H).

### Example 10

### 10-(azocan-1-ylmethyl)-3,4,6,11-tetrahydro-1H-indeno[1,2-c]isoquinolin-5(2H)-one

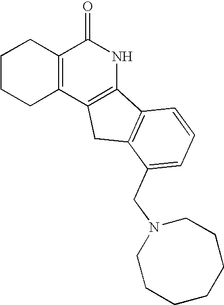

¹H NMR (DMSO-d6): d 12.22 (bs, 1H); 7.93 (m, 1H); 7.32 (m, 2H); 3.66 (s, 2H); 3.62 (s, 2H); 2.63 (m, 2H); 2.52 (m, 4H); 2.40 (m, 2H); 1.71 (m, 4H); 1.43 (m, 10H).

### Example 11

### 10-((4-hydroxypiperidin-1-yl)methyl)-3,4,6,11-tetrahydro-1H-indeno[1,2-c]isoquinolin-5(2H)-one mesylate

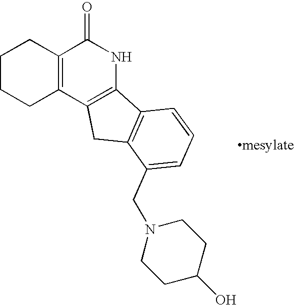

¹H NMR (DMSO-d6): d 12.23 (bs, 1H), 9.34 (bs, 1H), 8.00 (d, J=6.3 Hz, 1H), 7.50 (m, 2H); 4.39 (m, 2H), 3.92 (m, 1H), 3.73 (m, 2H), 3.63 (m, 1H); 3.23 (m, 4H), 2.64 (m, 2H); 2.37 (s, 2H); 2.33 (s, 3H); 1.91 (m, 2H) 1.71 (m, 4H), 159 (m, 2H).

### Example 12

### 10-((4,4-difluoropiperidin-1-yl)methyl)-3,4,6,11-tetrahydro-1H-indeno[1,2-c]isoquinolin-5(2H)-one

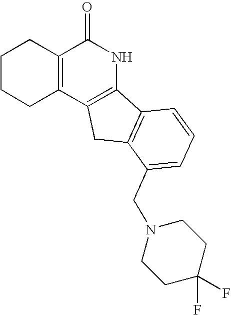

¹H NMR (DMSO-d6): d 12.21 (bs, 1H), 7.87 (d, J=7.5 Hz, 1H); 7.31 (m, 2H); 3.64 (s, 2H), 3.61 (s, 2H), 2.65 (m, 2H); 2.48 (m, 4H); 2.40 (m, 2H), 1.94 (m, 4H), 1.71 (m, 4H).

### Example 13

### 10-(thiazolidin-3-ylmethyl)-3,4,6,11-tetrahydro-1H-indeno[1,2-c]isoquinolin-5(2H)-one

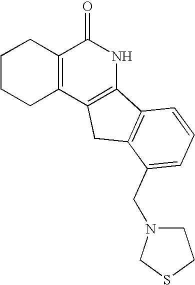

¹H NMR (DMSO-d6): d 12.28 (bs, 1H), 7.87 (d, J=7.5 Hz, 1H); 7.34 (m, 2H), 4.00 (s, 2H); 3.62 (m, 4H); 3.02 (m, 2H); 2.64 (m, 2H); 2.40 (m, 2H); 1.71 (m, 4H).

### Example 14

### 10-((4-ethoxypiperidin-1-yl)methyl)-3,4,6,11-tetrahydro-1H-indeno[1,2-c]isoquinolin-5(2H)-one mesylate

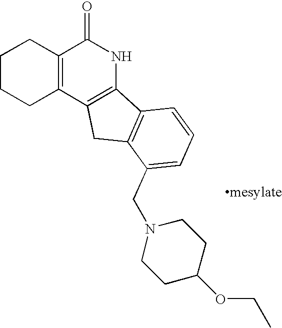

¹H NMR (DMSO-d6): d 12.26 (bs, 1H); 10.64 (bs, 1H); 7.99 (d, J=7.2 Hz, 1H); 7.66 (m, 1H); 7.46 (m, 1H); 4.38 (s, 1H); 3.85 (d, J=7.2 Hz, 2H) 3.63 (m, 1H); 3.44 (m, 2H); 3.17 (m, 4H); 2.64 (m, 2H); 2.40 (m, 2H); 2.30 (s, 3H); 2.03 (m, 2H); 1.92 (m, 2H); 1.72 (m, 4H); 1.07 (m, 3H).

### Example 15

### (S)-10-((3-fluoropyrrolidin-1-yl)methyl)-3,4,6,11-tetrahydro-1H-indeno[1,2-c]isoquinolin-5(2H)-one

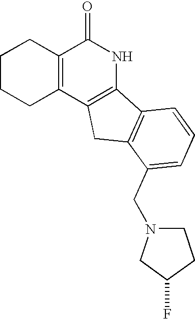

¹H NMR (DMSO-d6): d 12.23 (bs, 1H); 7.84 (d, J=7.5 Hz, 1H); 7.32 (m, 2H); 5.23 (d, J=53.8 Hz, 1H); 3.72 (s, 2H); 3.58 (s, 2H); 2.78 (m, 2H); 2.64 (m, 3H); 2.38 (m, 3H); 2.20 (m, 1H); 1.91 (m, 1H); 1.71 (m, 4H).

### Example 16

### (R)-10-((3-fluoropyrrolidin-1-yl)methyl)-3,4,6,11-tetrahydro-1H-indeno[1,2-c]isoquinolin-5(2H)-one mesylate

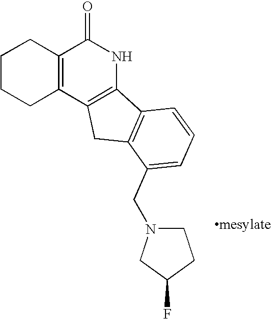

¹H NMR (DMSO-d6): d 12.21 (bs, 1H); 11.80 (bs, 1H); 8.00 (d, J=7.5 Hz, 1H); 7.67 (m, 1H); 7.45 (t, J=7.2 Hz, 1H); 5.41 (d, J=53.4 Hz, 1H); 4.50 (s, 2H); 3.85 (s, 2H); 3.38 (m, 4H); 2.62 (m, 2H); 2.39 (m, 2H); 2.33 (s, 3H); 2.30 (m, 2H); 1.71 (m, 4H).

### Example 17

### (S)-10-((3-fluoropyrrolidin-1-yl)methyl)-3,4,6,11-tetrahydro-1H-indeno[1,2-c]isoquinolin-5(2H)-one mesylate

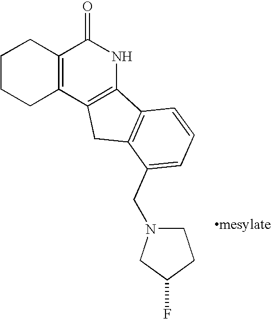

¹H NMR (DMSO-d6): d 10.60 (bs, 1H); 10.48 (bs, 1H); 8.00 (d, J=7.2 Hz, 1H); 7.56 (m, 1H); 7.48 (t, J=7.5 Hz, 1H); 5.43 (d, J=53.4 Hz, 1H); 4.55 (s, 2H); 3.77 (s, 2H); 3.60 (m, 2H); 3.41 (m, 2H); 2.64 (m, 2H); 2.31 (m, 5H); 2.20 (m, 2H); 1.72 (m, 4H).

### Example 18

### (S)-10-((3-fluoropyrrolidin-1-yl)methyl)-3,4,6,11-tetrahydro-1H-indeno[1,2-c]isoquinolin-5(2H)-one hydrochloride

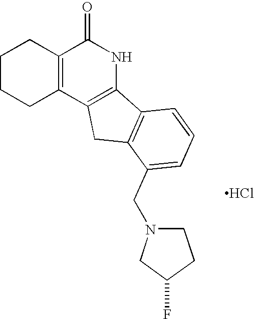

¹H NMR (DMSO-d6): d 11.63 (bs, 1H); 11.30 (bs, 1H); 8.00 (d, J=7.8 Hz, 1H); 7.66 (t, J=7.2 Hz, 1H); 7.48 (m, 1H); 5.43 (d, J=53.7 Hz, 1H); 4.51 (s, 2H); 3.82 (s, 2H); 3.45 (m, 4H); 2.64 (m, 2H); 2.01 (m, 2H); 2.25 (m, 2H); 1.72 (m, 4H).

### Example 19

### 10-(pyrrolidin-1-ylmethyl)-3,4,6,11-tetrahydro-1H-indeno[1,2-c]isoquinolin-5(2H)-one mesylate

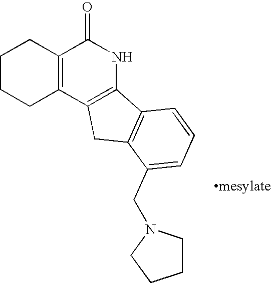

¹H NMR (DMSO-d6): d 9.81 (bs, 1H); 8.00 (dd, J=1.8 Hz, 6.6 Hz, 1H); 7.50 (m, 2H); 4.48 (d, J=5.4 Hz, 2H); 3.75 (s, 2H); 3.44 (m, 2H); 3.18 (m, 2H); 2.64 (m, 2H); 2.39 (m, 2H); 2.31 (s, 3H); 2.05 (m, 2H); 2.18 (m, 2H); 1.72 (m, 4H).

### Example 20

### 10-((4-methylpiperidin-1-yl)methyl)-3,4,6,11-tetrahydro-1H-indeno[1,2-c]isoquinolin-5(2H)-one mesylate

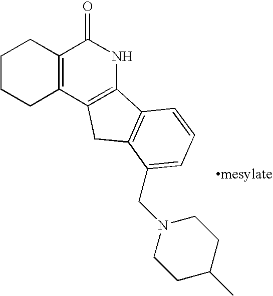

¹H NMR (DMSO-d6): d 12.24 (bs, 1H); 9.28 (bs, 1H); 8.09 (d, J=3.6 Hz, 1H); 7.51 (m, 2H); 4.38 (m, 2H); 3.74 (s, 2H); 3.34 (m, 2H); 3.07 (m, 2H); 2.65 (m, 2H); 2.40 (m, 2H); 2.32 (s, 3H); 1.78 (m, 1H); 1.72 (m, 4H); 1.58 (m, 1H); 1.35 (m, 2H); 1.04 (m, 1H); 0.89 (d, J=6.0 Hz, 3H).

### Example 21

### 10-(1,4′-bipiperidin-1′-ylmethyl)-3,4,6,11-tetrahydro-1H-indeno[1,2-c]isoquinolin-5(2H)-one

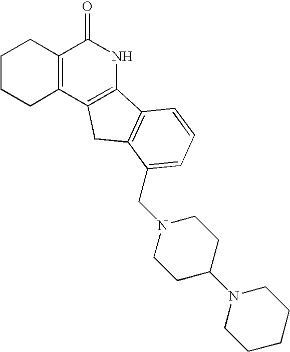

¹H NMR (DMSO-d6): d 12.23 (bs, 1H); 7.84 (d, J=7.5 Hz, 1H); 7.32 (t, J=7.2 Hz, 1H); 7.25 (d, J=7.2 Hz, 1H); 3.58 (s, 2H); 3.53 (s, 2H); 2.82 (m, 2H); 2.64 (m, 2H); 2.48 (m, 4H); 2.40 (m, 4H); 2.15 (m, 1H); 1.94 (m, 2H); 1.72 (m, 4H); 1.62 (m, 2H); 1.44 (m, 4H); 1.35 (m, 2H).

### Example 22

### 10-(1,4′-bipiperidin-1′-ylmethyl)-3,4,6,11-tetrahydro-1H-indeno[1,2-c]isoquinolin-5(2H)-one dihydrochloride

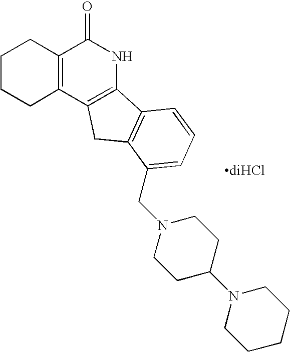

¹H NMR (D₂O): d 7.49 (d, J=7.5 Hz, 1H); 7.35 (t, J=7.5 Hz, 1H); 7.28 (d, J=7.5 Hz, 1H); 4.22 (s, 2H); 3.63 (d, J=12.0 Hz, 2H); 3.40 (m, 3H); 3.26 (s, 2H); 3.12 (t, J=12.0 Hz, 2H); 2.89 (t, J=12.0 Hz, 2H); 2.42 (m, 2H); 2.26 (d, J=12.9 Hz, 2H); 2.07 (m, 2H); 1.90 (m, 4H); 1.55 (m, 7H); 1.33 (m, 1H).

### Example 23

### 10-((4-(pyrrolidin-1-yl)piperidin-1-yl)methyl)-3,4,6,11-tetrahydro-1H-indeno[1,2-c]isoquinolin-5(2H)-one

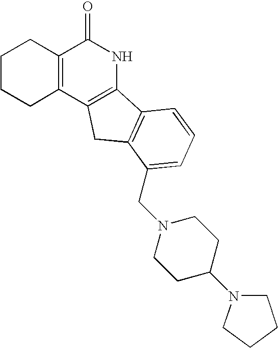

¹H NMR (DMSO-d6): d 12.23 (bs, 1H); 7.84 (d, J=7.5 Hz, 1H); 7.32 (t, J=7.5 Hz, 1H); 7.25 (d, J=7.5 Hz, 1H); 3.58 (s, 2H); 3.54 (s, 2H); 2.74 (m, 2H); 2.64 (m, 2H); 2.43 (m, 6H); 2.00 (m, 3H); 1.72 (m, 6H); 1.62 (m, 2H); 1.63 (m, 4H); 1.39 (m, 2H).

### Example 24

### 10-((4-(pyrrolidin-1-yl)piperidin-1-yl)methyl)-3,4,6,11-tetrahydro-1H-indeno[1,2-c]isoquinolin-5(2H)-one dimesylate

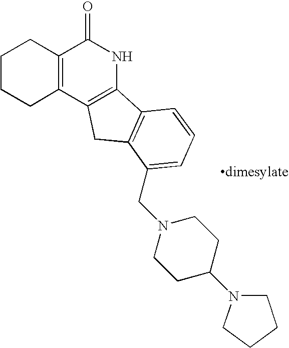

¹H NMR (D₂O): d 7.45 (d, J=7.2 Hz, 1H); 7.33 (t, J=7.2 Hz, 1H); 7.26 (d, J=7.2 Hz, 1H); 4.19 (s, 2H); 3.55 (m, 4H); 3.39 (m, 1H); 3.22 (s, 2H); 3.02 (m, 4H); 2.64 (s, 6H); 2.39 (m, 2H); 2.30 (m, 2H); 2.02 (m, 4H); 1.85 (m, 4H); 1.52 (m, 4H).

### Example 25

### 10-(1,4′-bipiperidin-1′-ylmethyl)-3,4,6,11-tetrahydro-1H-indeno[1,2-c]isoquinolin-5(2H)-one dimesylate

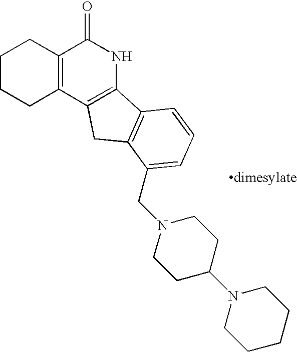

¹H NMR (D₂O): d 7.34 (d, J=7.2 Hz, 1H); 7.27 (t, J=7.2 Hz, 1H); 7.22 (d, J=7.2 Hz, 1H); 4.12 (s, 2H); 3.58 (d, J=12.0 Hz, 2H); 3.38 (m, 3H); 3.11 (m, 4H); 3.87 (m, 2H); 2.63 (s, 6H); 2.27 (m, 4H); 1.87 (m, 6H); 1.54 (m, 8H).

### Example 26

### 10-((cyclopentylamino)methyl)-3,4,6,11-tetrahydro-1H-indeno[1,2-c]isoquinolin-5(2H)-one

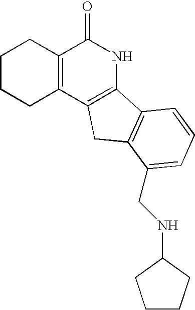

¹H NMR (DMSO-d₆): d 9.05 (bs, 2H), 7.98 (d, J=6.0 Hz, 1H), 7.51 (m, 2H), 4.25 (m, 2H), 3.85 (m, 5H), 3.65 (m, 2H), 3.28 (m, 2H), 2.68 (m, 2H), 2.42 (m, 2H), 2.03 (m, 2H), 1.72 (m, 2H), 1.58 (m, 2H).

MS: m/z 335.3 (M-H⁺).

### Example 27

### 10-((2-methoxyethylamino)methyl)-3,4,6,11-tetrahydro-1H-indeno[1,2-c]isoquinolin-5(2H)-one

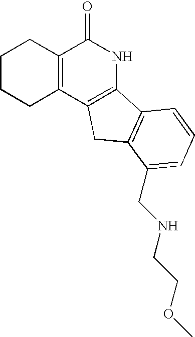

¹H NMR (DMSO-d₆): d 8.02 (m, 1H), 7.55 (m, 2H), 4.35 (m, 2H), 3.78 (m, 2H), 3.63 (m, 2H), 3.38 (s, 3H), 3.25 (m, 4H), 2.63 (m, 2H), 2.43 (m, 2H), 1.75 (m, 2H).

MS: m/z 325.1 (M-H⁺).

### Example 28

### 10-((dimethylamino)methyl)-3,4,6,11-tetrahydro-4H-indeno[1,2-c]isoquinolin-5(2H)-one)

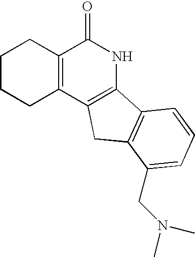

¹H NMR (DMSO-d6): d 8.05 (d, J=8.0 Hz, 1H), 7.63 (m, 2H), 4.45 (s, 2H), 3.80 (s, 2H), 3.32 (m, 2H), 2.82 (m, 6H), 2.65 (m, 2H), 2.42 (m, 2H), 1.78 (m, 2H).

MS: m/z 295.2 (M-H⁺).

### Example 29

### 10-(hydroxymethyl)-3,4,6,11-tetrahydro-1H-indeno[1,2-c]isoquinolin-5(2H)-one

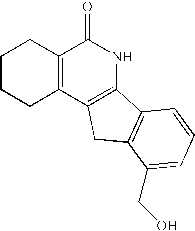

¹H NMR (DMSO-d₆): d 12.35 (bs, 1H), 7.90 (m, 1H), 7.38 (m, 2H), 4.63 (s, 2H), 3.55 (s, 2H), 3.18 (s, 2H), 2.63 (m, 2H), 2.43 (m, 2H), 1.75 (m, 2H).

MS: m/z 268.2 (M-H⁺).

### Example 30

### methyl 5-oxo-2,3,4,5,6,11-hexahydro-1H-indeno[1,2-c]isoquinoline-9-carboxylate

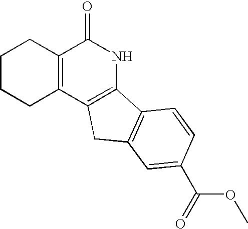

¹H NMR (DMSO-d₆): d 12.42 (bs, 1H), 8.17 (s, 1H), 8.05 (m, 2H), 3.87 (s, 2H), 3.68 (s, 2H), 3.31 (m, 2H), 2.64 (m, 2H), 2.43 (m, 2H), 1.75 (m, 2H).

MS: m/z 296.2 (M-H⁺).

### Example 31

### 10-(piperazin-1-ylmethyl)-3,4,6,11-tetrahydro-1H-indeno[1,2-c]isoquinolin-5(2H)-one

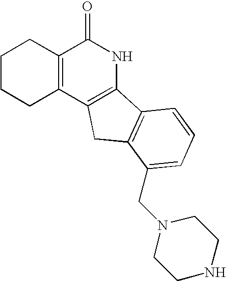

MS: m/z 322.2 (M-H⁺).

### Example 32

### 10-(2,6-dimethylmorpholino)-3,4,6,11-tetrahydro-1H-indeno[1,2-c]isoquinolin-5(2H)-one

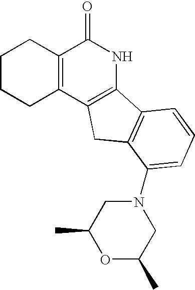

MS: m/z 351.3 (M-H⁺).

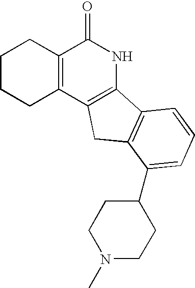

MS: m/z 331.2 (M-H³⁰.

### Example 33

### 9-(hydroxymethyl)-3,4,6,11-tetrahydro-1H-indeno[1,2-c]isoquinolin-5(2H)-one

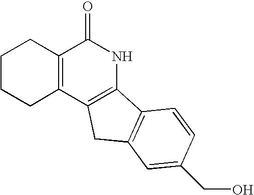

MS: m/z 368.2 (M-H⁺).

### Example 34

### 9-((4-propylpiperazin-1-yl)methyl)-3,4,6,11-tetrahydro-M-indeno[1,2-c]isoquinolin-5(2H)-one

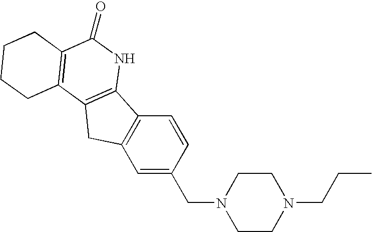

¹H NMR (DMSO-d₆): d 11.18 (bs, 1H), 9.28 (bs, 2H), 8.00 (d, J=6.0 Hz, 1H), 7.78 (d, J=6.0 Hz, 1H), 7.62 (m, 1H), 4.41 (m, 2H), 3.73 (m, 2H), 3.65 (m, 2H), 3.58 (m, 2H), 3.52 (m, 2H), 3.38 (m, 4H), 3.21 (m, 2H), 2.68 (m, 2H), 2.43 (m, 2H), 1.73 (m, 4H), 0.9 (m, 3H)

MS: m/z 378.3 (M-H⁺).

### Example 35

### 9-((4-(2-methoxyethyl)piperazin-1-yl)methyl)-3,4,6,11-tetrahydro-1H-indeno[1,2-c]isoquinolin-5(2H)-one

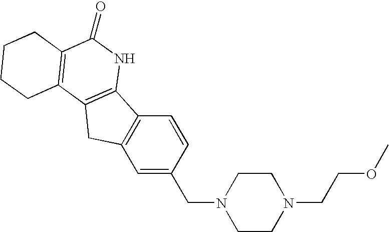

MS: m/z 394.3 (M-H⁺).

### Example 36

### 9-((2-methoxyethylamino)methyl)-3,4,6,11-tetrahydro-1H-indeno[1,2-c]isoquinolin-5(2H)-one

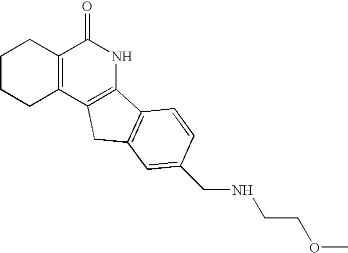

¹H NMR (DMSO-d6): d 9.08 (bs, 2H), 7.88 (d, J=6.0 Hz, 1H), 7.72 (s, 1H), 7.52 (m, 1H), 4.22 (t, J=6.0 Hz, 2H), 3.65 (s, 2H), 3.60 (m, 2H), 3.30 (s, 3H), 3.08 (m, 4H), 2.68 (m, 2H), 2.42 (m, 2H), 1.73 (m, 2H).

MS: m/z 325.3 (M-H⁺)

### Example 37

### 9-(morpholinomethyl)-3,4,6,1′-tetrahydro-4H-indeno[1,2-c]isoquinolin-5(2H)-one

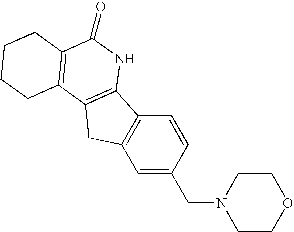

¹H NMR (DMSO-d6): d 11.15 (bs, 1H), 8.05 (m, 1H), 7.77 (s, 1H), 7.58 (m, 1H), 4.38 (m, 2H), 3.92 (m, 2H), 3.85 (m, 2H), 3.65 (s, 2H), 3.25 (m, 2H) 3.10 (m, 4H), 2.68 (m, 2H), 2.43 (m, 2H), 1.73 (m, 2H).

MS: m/z 337.2 (M-H⁺)

### Example 38

### 9-((4-hydroxypiperidin-1-yl)methyl)-3,4,6,11-tetrahydro-1H-indeno[1,2-c]isoquinolin-5(2H)-one

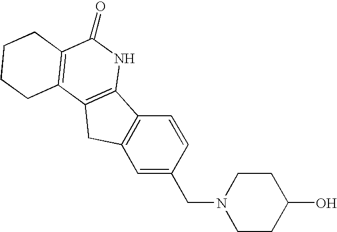

¹H NMR (DMSO-d6): d 10.18 (bs, 1H), 7.98 (d, J=9.0 Hz, 1H), 7.72 (d, J=9.0 Hz, 1H), 7.52 (m, 1H), 4.36 (m, 2H), 3.92 (m, 2H), 3.65 (m, 2H), 3.32 (m, 2H), 3.18 (m, 2H), 2.92 (m, 1H), 2.68 (m, 2H), 2.42 (m, 2H), 1.92 (m, 2H), 1.73 (m, 4H).

MS: m/z 351.3 (M-H⁺)

### Example 39

### 9-(pyrrolidin-1-ylmethyl)-3,4,6,11-tetrahydro-1H-indeno[1,2-c]isoquinolin-5(2H)-one

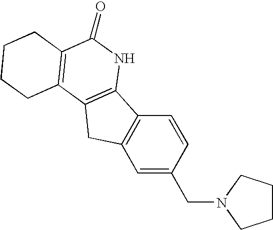

¹H NMR (DMSO-d6): d 12.39 (bs, 1H), 9.83 (bs, 1H), 7.95 (d, J=9.0 Hz, 1H), 7.72 (d, J=9.0 Hz, 1H), 7.52 (m, 1H), 4.42 (m, 2H), 3.65 (s, 2H), 3.13 (m, 2H), 2.65 (m, 2H), 2.42 (m, 2H), 2.05 (m, 2H), 1.83 (m, 2H), 1.73 (m, 2H), 1.18 (m, 4H)

MS: m/z 321.3 (M-H⁺)

### Example 40

### 9-((cyclopentylamino)methyl)-3,4,6,11-tetrahydro-1H-indeno[1,2-c]isoquinolin-5(2H)-one

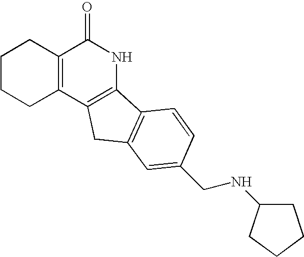

¹H NMR (DMSO-d₆): d 8.85 (bs, 2H), 7.88 (d, J=6.0 Hz, 1H), 7.72 (s, 1H), 7.54 (m, 1H), 4.22 (m, 2H), 3.95 (m, 1H), 3.65 (m, 2H), 3.52 (m, 4H), 3.15 (m, 2H), 2.65 (m, 2H), 2.42 (m, 2H), 2.00 (m, 2H), 1.73 (m, 2H), 1.53 (m, 2H)

MS: m/z 335.30 (M-H⁺).

### Example 41

### 10-(4-propylpiperazin-1-yl)-3,4,6,11-tetrahydro-1H-indeno[1,2-c]isoquinolin-5(2H)-one

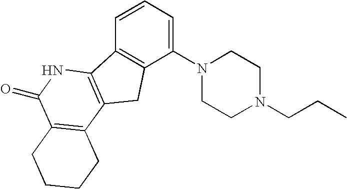

¹H NMR (DMSO-d6): d 9.55 (bs, 1H), 7.73 (d, J=8.0 Hz, 1H), 7.38 (m, 1H), 7.03 (d, J=8.0 Hz, 1H), 4.10 (m, 2H), 3.62 (m, 2H), 3.58 (m, 4H), 3.23 (m, 4H), 3.15 (m, 2H), 2.92 (m, 2H), 2.65 (m, 2H), 2.42 (m, 2H), 1.72 (m, 2H), 1.48 (t, J=7.0 Hz, 3H).

MS: m/z 364.30 (M-H⁺).

### Example 42

### 9-(4-butylpiperazin-1-yl)-3,4,6,11-tetrahydro-1H-indeno[1,2-c]isoquinolin-5(2H)-one

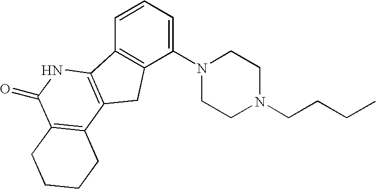

¹H NMR (DMSO-d6): d 12.19 (bs, 1H), 10.45 (bs, 1H), 7.72 (d, J=8.0 Hz, 1H), 7.38 (m, 1H), 7.15 (d, J=8.0 Hz, 1H), 3.62 (m, 6H), 3.25 (m, 6H), 3.15 (m, 2H), 2.65 (m, 2H), 2.42 (m, 2H), 1.75 (m, 4H), 1.38 (m, 2H), 0.95 (t, J=7.0 Hz, 3H).

MS: m/z 378.30 (M-H⁺).

### Example 43

### 10-(4-(2-methoxyethyl)piperazin-1-yl)-3,4,6,11-tetrahydro-1H-indeno[1,2-c]isoquinolin-5(2H)-one

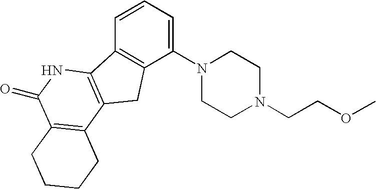

¹H NMR (DMSO-d6): d 9.65 (bs, 1H), 7.73 (d, J=8.0 Hz, 1H), 7.38 (m, 1H), 7.16 (d, J=8.0 Hz, 1H), 3.72 (m, 4H), 3.63 (s, 3H), 3.53 (s, 2H), 3.42 (m, 4H), 3.25 (m, 4H), 3.15 (m, 2H), 2.65 (m, 2H), 2.43 (m, 2H), 1.75 (m, 2H).

MS: m/z 380.30 (M-H⁺).

### Example 44

### 9-((4-butylpiperazin-1-yl)methyl)-3,4,6,11-tetrahydro-4H-indeno[1,2-c]isoquinolin-5(2H)-one

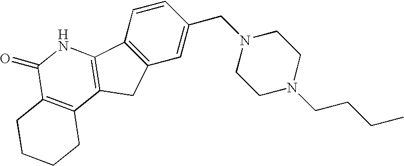

¹H NMR (DMSO-d6): d 12.39 (bs, 1H), 9.63 (bs, 1H), 8.00 (m, 1H), 7.78 (m, 1H), 7.60 (m, 1H), 4.22 (m, 2H), 3.66 (s, 2H), 3.35 (m, 2H), 3.09 (m, 10H), 2.65 (m, 2H), 2.43 (m, 2H), 1.65 (m, 2H), 1.33 (m, 2H), 1.20 (m, 2H), 0.90 (m, 3H).

MS: m/z 392.40 (M-H⁺).

### Example 45

### 9-((2-morpholinoethylamino)methyl)-3,4,6,11-tetrahydro-1H-indeno[1,2-c]isoquinolin-5(2H)-one

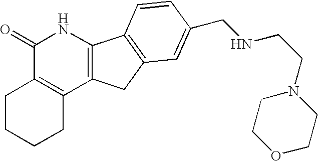

¹H NMR (DMSO-d6): d 8.00 (d, J=8.0 Hz, 1H), 7.65 (s, 1H), 7.43 (d, J=8.0 Hz, 1H), 4.22 (m, 2H), 3.55 (m, 10H), 3.16 (m, 6H), 2.62 (m, 2H), 2.43 (m, 2H), 1.75 (m, 2H).

MS: m/z 380.30 (M-H⁺).

### Example 46

### 10-(2-aminopropan-2-yl)-3,4,6,11-tetrahydro-1H-indeno[1,2-c]isoquinolin-5(2H)-one

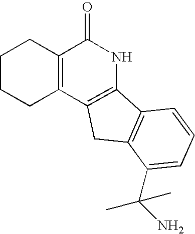

¹HNMR (DMSO-d6): d 12.38 (bs, 1H), 8.63 (bs, 3H), 8.05 (d, J=8.0 Hz, 1H), 7.53 (m, 1H), 7.40 (d, J=8.0 Hz, 1H), 3.92 (s, 2H), 3.19 (m, 2H), 2.62 (m, 2H), 2.43 (m, 2H), 1.78 (m, 6H) 1.68 (m, 2H).

MS: m/z 295.20 (M-H⁺).

### Example 47

### 9-((4-(trifluoromethyl)piperidin-1-yl)methyl)-5,10-dihydro-4H-indeno[1,2-b]thieno[3,2-d]pyridin-4-one

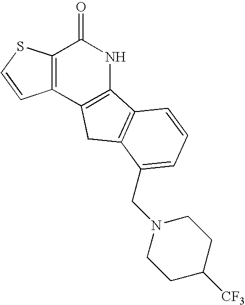

¹H NMR (DMSO-d6): d 12.62 (bs, 1H), 9.63 (bs, 1H), 8.23 (s, 1H), 8.16 (s, 1H), 7.58 (m, 2H), 7.42 (m, 1H), 4.51 (m, 2H), 4.05 (s, 2H), 3.52 (m, 2H), 3.17 (m, 2H), 2.73 (m, 1H), 2.03 (m, 2H), 1.75 (m, 2H).

MS: m/z 405.20 (M-H⁺).

### Example 48

### 9-((cyclopentylamino)methyl)-5,10-dihydro-4H-indeno[1,2-b]thieno[3,2-d]pyridin-4-one

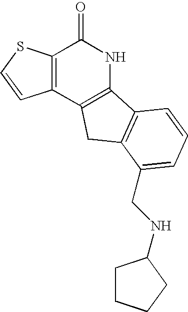

¹H NMR (DMSO-d6): d 12.62 (bs, 1H), 8.83 (bs, 2H), 8.15 (s, 1H), 8.05 (s, 1H), 7.58 (m, 3H), 4.23 (m, 2H), 4.05 (s, 2H), 3.64 (m, 1H), 2.03 (m, 2H), 1.75 (m, 4H), 1.58 (m, 2H).

MS: m/z 337.20 (M-H⁺).

### Example 49

### 10-(4-(2-methoxyethyl)piperazin-1-yl)-6,11-dihydro-5H-indeno[1,2-h][1,6]naphthyridin-5-one

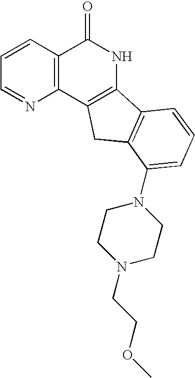

MS: m/z 377.2 (M-H⁺).

### Example 50

### 10-((1-(hydroxymethyl)cyclopentylamino)methyl)-3,4,6,11-tetrahydro-1H-indeno[1,2-c]isoquinolin-5(2H)-one

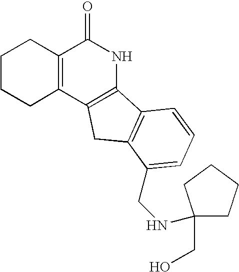

¹H NMR (DMSO-d6): d 8.93 (bs, 2H), 8.05 (m, 1H), 7.58 (m, 2H), 4.23 (m, 2H), 3.89 (m, 4H), 3.79 (m, 2H), 3.60 (m, 2H), 3.19 (m, 2H), 2.63 (m, 2H), 2.43 (m, 2H), 1.78 (m, 2H) 1.64 (m, 4H).

MS: m/z 365.30 (M-H⁺).

### Example 51

### 2-hydroxy-2-methyl-N-(5-oxo-2,3,4,5,6,11-hexahydro-1H-indeno[1,2-c]isoquinolin-10-yl)propanamide

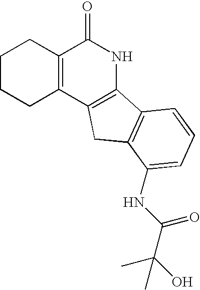

¹H NMR (DMSO-d6): d 9.36 (bs, 1H), 7.78 (m, 2H), 7.41 (m, 1H), 5.93 (bs, 1H), 5.73 (bs, 1H), 3.56 (m, 2H), 3.19 (m, 2H), 2.62 (m, 2H), 2.42 (m, 2H), 1.78 (m, 2H), 1.40 (m, 6H).

MS: m/z 339.20 (M-H⁺).

### Example 52

### 3-(methylthio)-N-(5-oxo-2,3,4,5,6,11-hexahydro-1H-indeno[1,2-c]isoquinolin-10-yl)propanamide

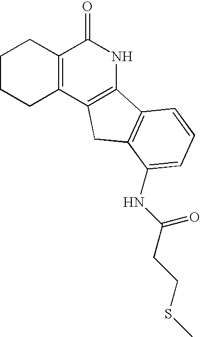

¹H NMR (DMSO-d6): d 9.76 (bs, 1H), 7.78 (m, 2H), 7.71 (m, 1H), 3.59 (s, 2H), 3.18 (m, 2H), 2.79 (m, 2H), 2.75 (m, 2H), 2.73 (m, 2H), 2.43 (m, 2H), 2.25 (s, 3H), 1.73 (m, 2H).

### Example 53

### 3-(methylsulfinyl)-N-(5-oxo-2,3,4,5,6,11-hexahydro-1H-indeno[1,2-c]isoquinolin-10-yl)propanamide

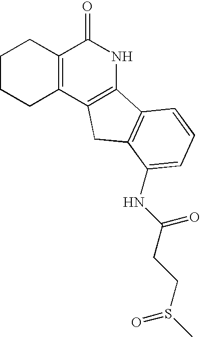

¹H NMR (DMSO-d6): d 9.84 (bs, 1H), 7.78 (m, 2H), 7.38 (m, 1H), 3.59 (s, 2H), 3.19 (m, 2H), 2.95 (m, 5H), 2.63 (m, 4H), 2.43 (m, 2H), 1.75 (m, 2H).

MS: m/z 371.2 (M-H⁺).

### Example 54

### 9-((4-(trifluoromethyl)piperidin-1-yl)methyl)-5,10-dihydro-4H-indeno[1,2-b]thieno[2,3-d]pyridin-4-one

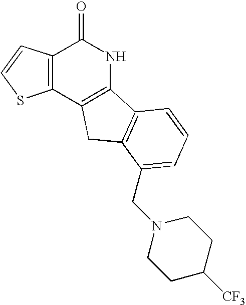

¹H NMR (DMSO-d₆): d 12.59 (bs, 1H), 9.53 (bs, 1H). 8.13 (d, J=2.0 Hz, 1H), 7.63 (d, J=2.0 Hz, 1H), 7.49 (m, 3H), 4.51 (s, 2H), 4.05 (s, 2H), 3.58 (d, J=8.0 Hz, 2H), 3.19 (m, 2H), 2.65 (m, 1H), 2.03 (m, 2H) 1.78 (m, 2H).

MS: m/z 405.2 (M-H⁺).

### Example 55

### 9-((cyclopentylamino)methyl)-5,10-dihydro-4H-indeno[1,2-b]thieno[2,3-d]pyridin-4-one

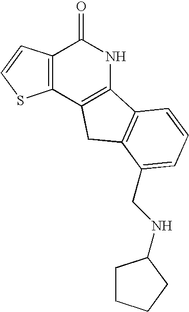

¹H NMR (DMSO-d6): d 8.82 (bs, 2H), 8.08 (d, J=6.0 Hz, 1H), 7.62 (d, J=6.0 Hz, 1H), 7.58 (m, 1H), 7.50 (m, 2H), 4.33 (m, 2H), 4.08 (s, 2H), 3.65 (m, 1H), 2.08 (m, 2H), 1.72 (m, 4H), 1.58 (m, 2H).

MS: m/z 337.2 (M-H⁺).

### Example 56

### 10-(4-methylpiperazin-1-yl)-6,11-dihydro-5H-indeno[1,2-d][1,6]naphthyridin-5-one

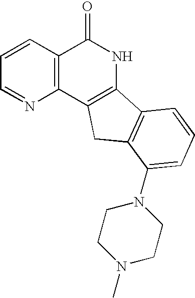

¹H NMR (DMSO-d6): d 8.92 (m, 1H), 8.52 (d, J=8.0 Hz, 1H), 7.73 (d, J=8.0 Hz, 1H), 7.42 (m, 1H), 7.38 (m, 1H), 7.05 (d, J=6.0 Hz, 1H), 3.92 (s, 2H), 3.15 (m, 4H), 2.78 (m, 4H), 2.32 (m, 3H).

MS: m/z 333.2 (M-H⁺).

### Example 57

### 10-(hydroxymethyl)-6,11-dihydro-5H-indeno[1,2-h][1,6]naphthyridin-5-one

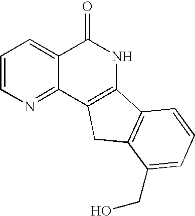

MS: m/z 265.1 (M-H⁺).

### Example 58

### 10-(4-butylpiperazin-1-yl)-6,11-dihydro-5H-indeno[1,2-h][1,6]naphthyridin-5-one

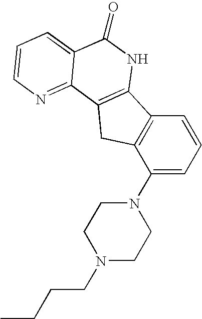

¹H NMR (DMSO-d6): d 12.59 (bs, 1H), 9.43 (bs, 1H), 8.94 (m, 1H), 8.52 (d, J=8.0 Hz, 1H), 7.81 (d, J=8.0 Hz, 1H), 7.42 (m, 2H), 7.05 (d, J=8.0 Hz, 1H), 3.92 (s, 2H), 3.65 (m, 4H), 3.45 (m, 4H), 3.15 (m, 2H), 1.70 (m, 2H), 1.62 (m, 2H), 0.98 (t, J=6.0 Hz, 3H).

MS: m/z 375.2 (M-H⁺).

### Example 59

### 10-(4-ethylpiperazin-1-yl)-6,11-dihydro-5H-indeno[1,2-h][1,6]naphthyridin-5-one

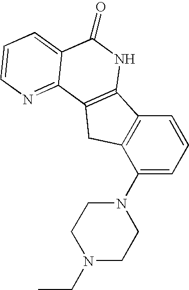

¹H NMR (DMSO-d6): d 12.62 (bs, 1H), 9.50 (bs, 1H), 8.92 (m, 1H), 8.52 (m, 1H), 7.88 (d, J=8.0 Hz, 1H), 7.56 (m, 1H), 7.48 (m, 1H), 7.05 (d, J=8.0 Hz, 1H), 3.92 (s, 2H), 3.65 (m, 4H), 3.35 (m, 4H), 3.25 (m, 2H), 1.37 (m, 3H).

MS: m/z 347.2 (M-H⁺).

### Example 60

### 10-(4-isopropylpiperazin-1-yl)-6,11-dihydro-5,1-indeno[1,2-h][1,6]naphthyridin-5-one

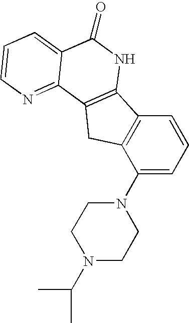

¹H NMR (DMSO-d6): d 12.62 (bs, 1H), 10.53 (bs, 1H), 8.92 (m, 1H), 8.52 (m, 1H), 7.88 (d, J=8.0 Hz, 1H), 7.56 (m, 1H), 7.48 (m, 1H), 7.05 (d, J=8.0 Hz, 1H), 3.92 (s, 2H), 3.65 (m, 4H), 3.55 (m, 4H), 3.25 (m, 1H), 1.35 (m, 6H).

MS: m/z 361.2 (M-H⁺).

### Example 61

### 10-(4-(cyclopropylmethyl)piperazin-1-yl)-6,11-dihydro-5H-indeno[1,2-h][1,6]naphthyridin-5-one

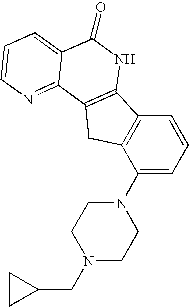

¹H NMR (DMSO-d6): d 12.57 (bs, 1H), 9.58 (bs, 1H), 8.95 (m, 1H), 8.54 (m, 1H), 7.86 (d, J=8.0 Hz, 1H), 7.52 (m, 2H), 7.05 (d, J=8.0 Hz, 1H), 3.96 (m, 2H), 3.60 (m, 6H), 3.25 (m, 2H), 3.15 (m, 2H), 1.15 (m, 1H), 0.65 (m, 2H), 0.25 (m, 2H).

MS: m/z 373.2 (M-H⁺).

### Example 62

### 10-((cyclopentylamino)methyl)-6,11-dihydro-5H-indeno[1,2-h][1,6]naphthyridin-5-one

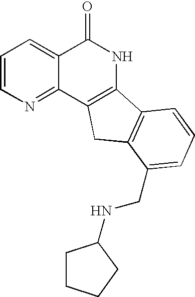

¹H NMR (DMSO-d6): d 9.08 (bs, 2H), 8.97 (m, 1H), 8.56 (d, J=7.0 Hz, 1H), 8.13 (d, J=7.0 Hz, 1H), 7.61 (d, J=7.0 Hz, 1H), 7.58 (m, 1H), 7.50 (m, 1H), 4.65 (m, 2H), 4.15 (m, 3H), 3.65 (m, 4H), 1.75 (m, 2H), 1.55 (m, 2H).

MS: m/z 332.2 (M-H⁺).

### Example 63

### methyl 5-oxo-2,3,4,5,6,11-hexahydro-1H-indeno[1,2-c]isoquinoline-10-carboxylate

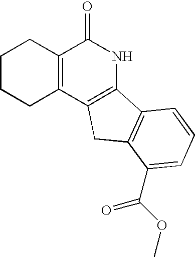

¹H NMR (DMSO-d6): 12.39 (bs, 1H), 8.20 (d, J=6.0 Hz, 1H), 7.91 (d, J=6.0 Hz, 1H), 7.59 (m, 1H), 3.91 (s, 3H), 3.85 (s, 2H), 3.10 (m, 2H), 2.65 (m, 2H), 2.42 (m, 2H), 1.72 (m, 2H)

MS: m/z 296.1 (M-H⁺).

### Example 64

### 9-((diethylamino)methyl)-3,4,6,11-tetrahydro-1H-indeno[1,2-c]isoquinolin-5(2H)-one

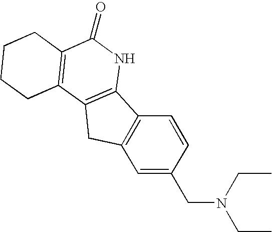

¹H NMR (DMSO-d6): d 12.23 (bs, 1H); 7.82 (d, J=7.5 Hz, 1H); 7.301 (m, 2H); 3.62 (s, 2H); 3.59 (s, 2H); 2.64 (m, 2H); 2.45 (m, 2H); 2.42 (m, 4H); 1.72 (m, 4H); 0.98 (m, 6H).

### Example 65

### 9-((4,4-difluoropiperidin-1-yl)methyl)-3,4,6,11-tetrahydro-1H-indeno[1,2-c]isoquinolin-5(2H)-one

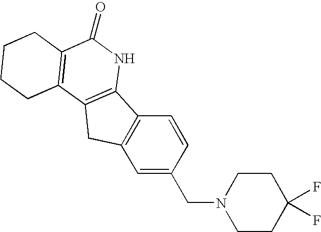

¹H NMR (DMSO-d6): d 12.23 (bs, 1H); 9.68 (bs, 1H); 8.03 (t, J=3.9 Hz, 1H); 7.53 (m, 2H); 4.54 (s, 2H), 3.76 (s, 2H); 3.48 (m, 2H); 3.35 (m, 2H); 2.66 (m, 2H); 2.42 (m, 2H); 2.35 (s, 3H); 2.28 (m, 4H); 1.73 (m, 4H).

### Example 66

### 9-((4-fluoropiperidin-1-yl)methyl)-3,4,6,11-tetrahydro-1H-indeno[1,2-c]isoquinolin-5(2H)-one

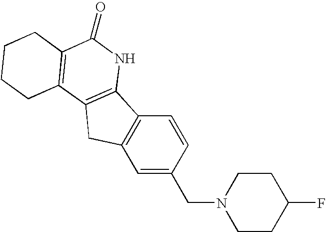

¹H NMR (DMSO-d6): d 12.17 (bs, 1H), 7.84 (d, J=7.5 Hz, 1H); 7.32 (t, J=7.5 Hz, 1H); 7.27 (t, J=7.5 Hz, 1H); 4.59 (m, 1H); 3.58 (s, 2H); 3.56 (s, 2H); 2.64 (m, 2H); 2.50 (m, 2H); 2.40 (m, 2H); 2.30 (m, 2H); 1.83 (m, 2H); 1.73 (m, 2H); 1.72 (m, 4H).

### Example 67

### 10-((diethylamino)methyl)-3,4,6,11-tetrahydro-1H-indeno[1,2-c]isoquinolin-5(2H)-one

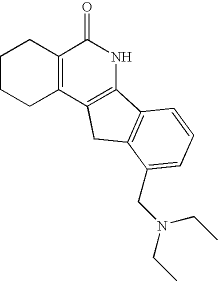

¹H NMR (DMSO-d6): d 10.74 (bs, 1H), 8.02 (d, J=7.8 Hz, 1H); 7.68 (d, J=7.8 Hz, 1H); 7.44-7.49 (dd, J=7.5 Hz and 3.3 Hz, 1H); 4.34-4.37 (m, 6H); 3.86 (s, 1H); 3.01-3.14 (m, 4H); 2.64 (s, 1H); 2.49 (m, 1H); 2.41 (s, 1H); 1.72 (s, 3H); 1.62-1.30 (m, 5H).

### Example 68

### 10-((4-fluoropiperidin-1-yl)methyl)-3,4,6,11-tetrahydro-1H-indeno[1,2-c]isoquinolin-5(2H)-one

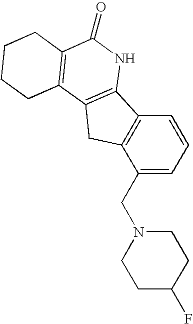

¹H NMR (DMSO-d6): d 12.22 (bs, 1H), 7.84 (d, J=7.5 Hz, 1H); 7.26-7.35 (m, 2H); 4.59 (m, 1H); 3.58 (s, 2H); 3.56 (s, 2H); 2.64 (m, 2H); 2.50 (m, 2H); 2.40 (m, 2H); 2.30 (m, 2H); 1.83 (m, 2H); 1.73 (m, 2H); 1.72 (m, 4H).

### Example 69

### 10-((5,6-dihydropyridin-1(2H)-yl)methyl)-3,4,6,11-tetrahydro-1H-indeno[1,2-c]isoquinolin-5(2H)-one

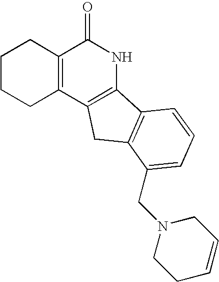

¹H NMR (DMSO-d6): d 12.23 (bs, 1H), 7.84 (d, J=7.5 Hz, 1H); 7.26-7.35 (m, 2H); 5.59-5.67 (m, 2H), 3.62 (s, 2H); 3.58 (s, 2H); 3.22-3.29 (m, 2H), 2.87 (s, 2H); 2.62 (s, 2H); 2.39 (s, 2H), 2.04 (s, 2H), 1.69 (s, 4H).

### Example 70

### 10-(thiomorpholinomethyl-1,1-dioxide)-3,4,6,11-tetrahydro-4H-indeno[1,2-c]isoquinolin-5(2H)-one

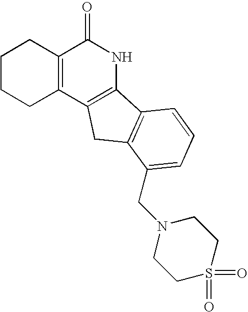

¹H NMR (DMSO-d6): d 12.26 (bs, 1H), 7.86 (d, J=7.5 Hz, 1H); 7.27-7.37 (m, 2H); 3.75 (s, 2H), 3.63 (s, 2H); 3.10 (s, 3H); 2.87 (s, 3H); 2.66 (s, 2H); 2.40 (s, 2H), 1.71 (s, 4H).

### Example I

### Determination of the Effect of Tetracyclic 1H-indeno[1,2-b]pyridine-2(5H)-one analogs on in vitro PARP Activity

The ability of an illustrative Tetracyclic 1H-indeno[1,2-b]pyridine-2(5H)-one analog to inhibit PARP and prevent peroxynitrite induced cytotoxicity can be demonstrated using methods described in Virag et al., Br. J. Pharmacol. 1999, 126(3):769-77; and Immunology 1998, 94(3):345-55.

The potency of inhibition of purified PARP enzyme can be subsequently determined for selected Tetracyclic 1H-indeno[1,2-b]pyridine-2(5H)-one analogs, and the potency is compared with that of 3-aminobenzamide, a prototypical benchmark PARP inhibitor. The assay is performed in 96 well ELISA plates according to instructions provided with a commercially available PARP inhibition assay kit (for example, from Trevigen, Gaithersburg, Md.).

### Example II

### Determination of the Effect of Tetracyclic 1H-indeno[1,2-b]pyridine-2(5H)-one analogs in an in vitro Model of Cell Death

Using an in vitro, oxidant-stimulated thymocyte assay (described, in detail, in Virag et al., Immunology 94(3):345-55, 1998), an illustrative Tetracyclic 1H-indeno[1,2-b]pyridine-2(5H)-one analog can be tested for its ability to prevent the oxidant-induced suppression of the viability of the cells and as such, this assay represents an in vitro model of reperfusion related cell death in ischemic organs.

### Example III

### Determination of the Effect of Tetracyclic 1H-indeno[1,2-b]pyridine-2(5H)-one analogs on in vivo Models of Inflammatory Diseases

The effect of an illustrative Tetracyclic 1H-indeno[1,2-b]pyridine-2(5H)-one analog can be determined using a systemic inflammatory model induced by bacterial lipopolysaccharide (LPS), which is reported to be responsible for causing reperfusion injurys and inflammatory diseases such as septic shock and systemic inflammatory response syndrome in animals (see Parrillo, N. Engl. J. Med., 328:1471-1478 (1993) and Lamping, J. Clin. Invest. 101:2065-2071 (1998).

### Example IV

### Determination of the Effect of Tetracyclic 1H-indeno[1,2-b]pyridine-2(5H)-one analogs on in vivo Models of Reperfusion injury

The efficacy of an illustrative Tetracyclic 1H-indeno[1,2-b]pyridine-2(5H)-one analog in a mouse model of ischemic and reperfused gut can be determined according to the method described in Liaudet et al., Shock 2000, 14(2):134-41.

In another set of experiments, the effect of an illustrative Tetracyclic 1H-indeno[1,2-b]pyridine-2(5H)-one analog in a rat model of middle cerebral artery occlusion/reperfusion can be assayed as described in Abdelkarim et al., Int. J. Mol. Med. 2001, 7(3):255-60.

### Example V

### Determination of the Effect of Tetracyclic 1H-indeno[1,2-b]pyridine-2(5H)-one analogs in an in vivo Model of Diabetes Mellitus

PARP inhibitors and PARP deficiency are known to reduce the development of diabetes mellitus and the incidence of diabetic complications. The anti-diabetic effect of an illustrative Tetracyclic 1H-indeno[1,2-b]pyridine-2(5H)-one analog can be determined using a single high-dose streptozotocin model of diabetes mellitus, which can be used as conducted as described in Mabley et al., Br. J. Pharmacol. 2001, 133(6):909-9; and Soriano et al., Nat. Med. 2001, 7(1):108-13. Briefly, 160 mg/kg streptozotocin is injected to mice treated with vehicle (control) or with an illustrative Tetracyclic 1H-indeno[1,2-b]pyridine-2(5H)-one analog intraperitoneally (3 mg/kg) and 3 days later blood sugar levels are determined using a blood glucose meter.

### Example VI

### Determination of the Effect of Tetracyclic 1H-indeno[1,2-b]pyridine-2(5H)-one analogs in an in vivo Model of Erectile Dysfunction

Experiments are conducted in male Sprague-Dawley rats according to previously published methods for forceps-induced nerve crush injury and erectile function measurements (Rehman, J., et al, Urology 51:640-644, 1998; Sezen, S. F., et al., Int. J. Impot. Res. 14:506-12, 2002). Subjects are anesthetized with Phenobarbital. The prostate of the subjects is exposed and the cavernosal nerve is clipped on either side with a forceps to induce mechanical injury (crush). This rat model mimics the nerve injury that develops during human male prostatectomy, leading to nerve injury and subsequent erectile dysfunction. Subjects are studied 2 weeks after the injury. Two groups of subjects are used, one group treated with vehicle and one group treated with an illustrative Tetracyclic 1H-indeno[1,2-b]pyridine-2(5H)-one analog. The illustrative Tetracyclic 1H-indeno[1,2-b]pyridine-2(5H)-one analog is injected at 30 mg/kg i.v. immediately before the crush injury, and on the following day at the same dose. Thereafter, for 12 days, subjects are treated with 60 mg/kg of the illustrative Tetracyclic 1H-indeno[1,2-b]pyridine-2(5H)-one analog intraperitoneally. At 2 weeks, subjects are re-anesthetized and measured for mean arterial blood pressure (MAP) and intracavernosal pressure (ICP). Cavernosal nerve stimulation is conducted at 5 and 7.5 V using a square pulse stimulator for 30 msec. ICP is determined as the area under curve (mmHg X sec). In addition, IPC/MAP ratios are determined.

### Example VII

### Effect of Tetracyclic 1H-indeno[1,2-b]pyridine-2(5H)-one analogs on the prophylaxis of contrast induced nephropathy

### CIN Animal Model

Male Wistar rats (150-200 g; Harlan Nossanr) were housed in a controlled environment and provided with standard rodent chow and water.

### Induction of Diabetes

After 12 h of fasting, the animals received a single 60 mg/kg intravenous (i.v.) injection of streptozotocin (Sigma, St. Louis, Mo.) in 10 mM sodium citrate buffer, pH 4.5 (see figure showing CIN model). Control non-diabetic animals were fasted and received citrate buffer alone. After 24 h, animals with blood glucose levels greater than 250 mg/dl were considered diabetic. All experiments were performed 10 days following the induction of diabetes. The diabetic state was evaluated daily by determination of the blood glucose levels. Upon the induction of the diabetic state the animals were presenting with a major risk factor pre-disposing the animals to CIN after the administration of contrast media.

### Contrast-Induced Nephropathy (CIN)

10 days following the induction of diabetes the rats were anesthetized with 90 mg/kg ketamine i.m. and 10 mg/kg xylazine i.m. and were treated with the contrast agent iomeprol (10 mL/kg injected via the lateral tail vein) or with 0.9% normal saline. The contrast agent used was the low osmolar non-ionic monomer iomeprol (lomeron, 400 mgI mL−1; Bracco SpA, Milan, Italy) with an osmolality of 726±34 mosmol/kg H₂O.

### Experimental Protocol

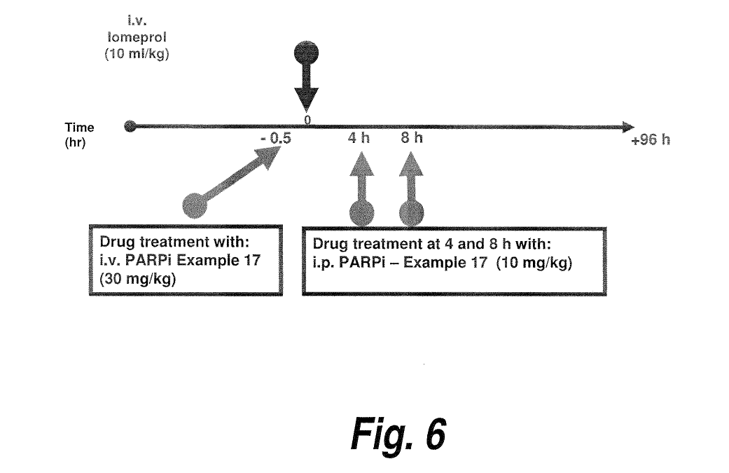

Upon completion of surgical procedures, the animals were randomly allocated to different groups. Contrast agent and drugs were administered as shown in FIG. 6.

### Measurement of Biochemical Parameters

At the indicated time point blood samples were collected via the lateral tail vein into S1/3 tubes containing serum gel. The samples were centrifuged (6000 r.p.m. for 3 min) to separate plasma. All plasma samples were analyzed for biochemical parameters within 24 hours after collection. Plasma urea, plasma and urine concentrations of creatinine were measured as indicators of impaired glomerular function. Plasma concentrations of NGAL (neutrophil gelatinase associated lipocalin) levels and urine αGST (alpha glutathione S-transferase)and NGAL levels were evaluated.

### Histologic Evaluation

At postmortem, a 5 μm section of kidney was removed and placed in formalin and processed through to wax. Five millimeter sections were cut and stained with hematoxylin and eosin. Histologic assessment of outer medulla damage was examined by an experienced morphologist, who was not aware of the sample identity. The criteria for injury/necrosis were the following: 0=normal histology; 1=minor edema, minor cell swelling; 2=haemorrhage, moderate edema, moderate cells vacuolization and swelling; 3=moderate haemorrhage, moderate edema, moderate cells vacuolization, swelling and chromatin alteration; 4=severe edema, severe cells vacuolization, swelling and chromatin alteration, presence of necrosis spot; 5=severe edema, severe cells vacuolization, swelling and chromatin alteration, severe necrosis.

### Materials

Unless otherwise stated, all compounds were obtained from Sigma-Aldrich Company Ltd. (Milan, Italy). All stock solutions were prepared in non-pyrogenic saline (0.9% NaCl; Baxter, Italy) or 10% DMSO.

### Results

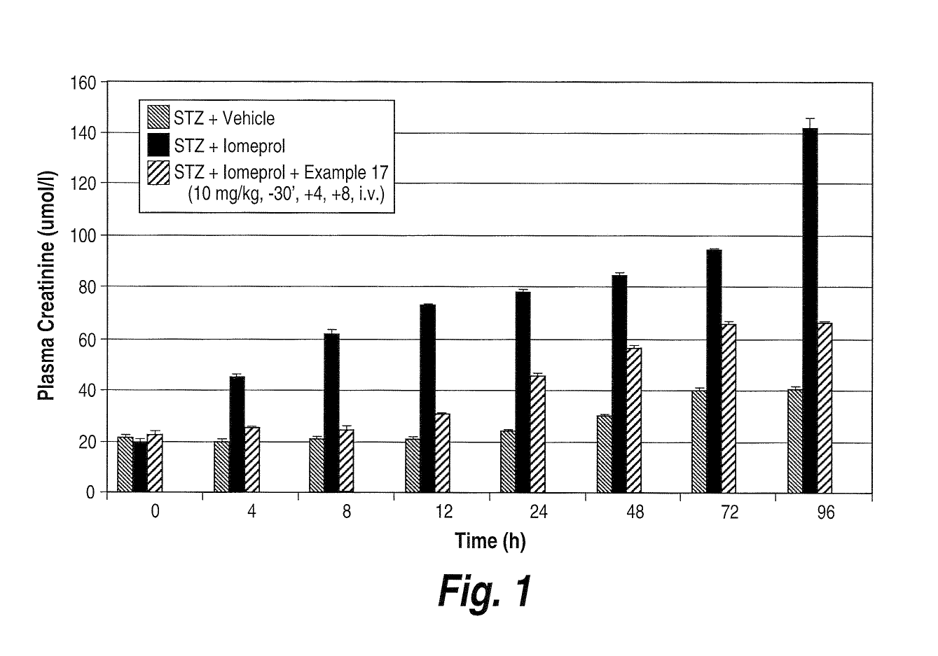

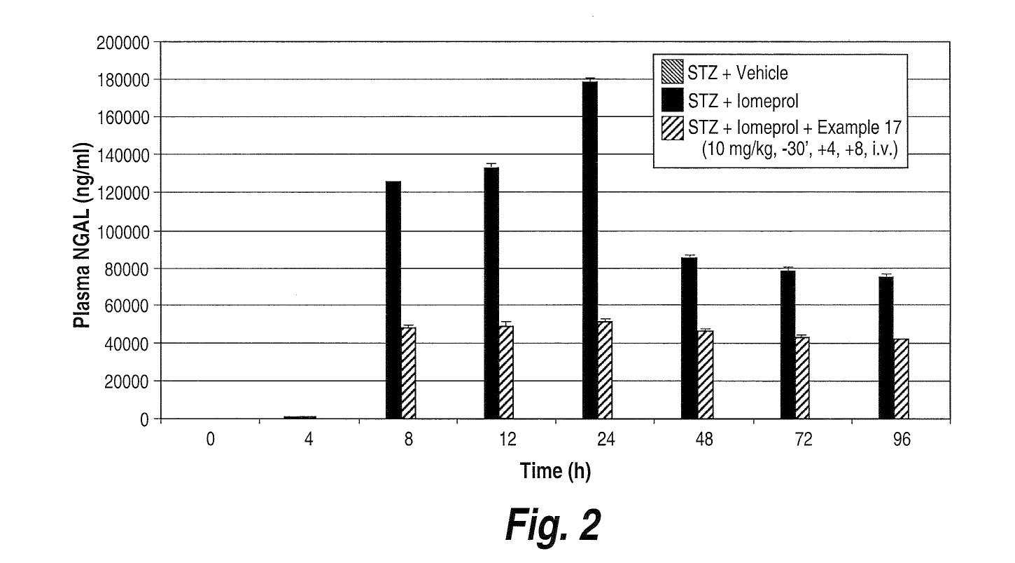

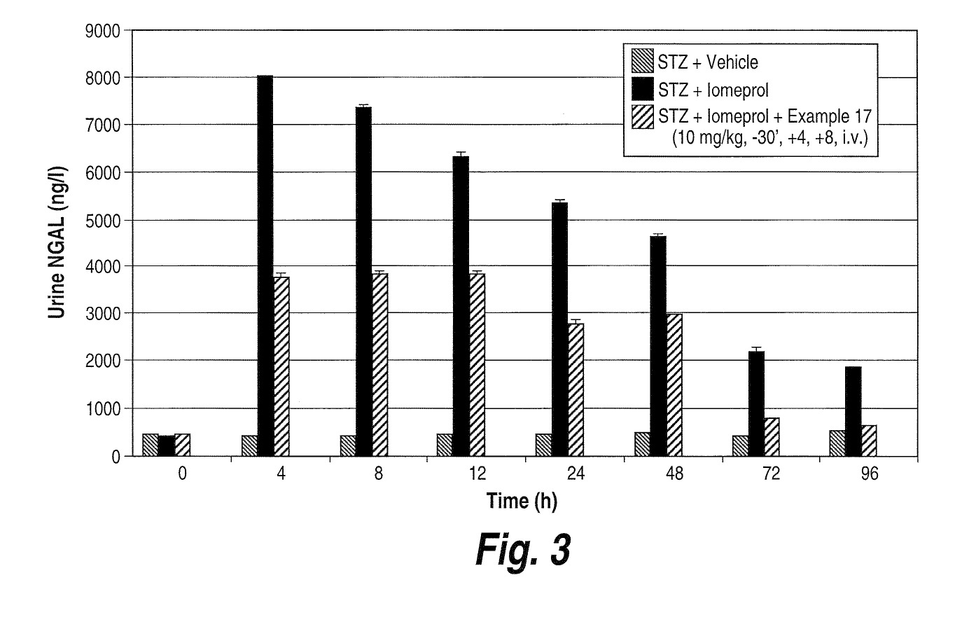

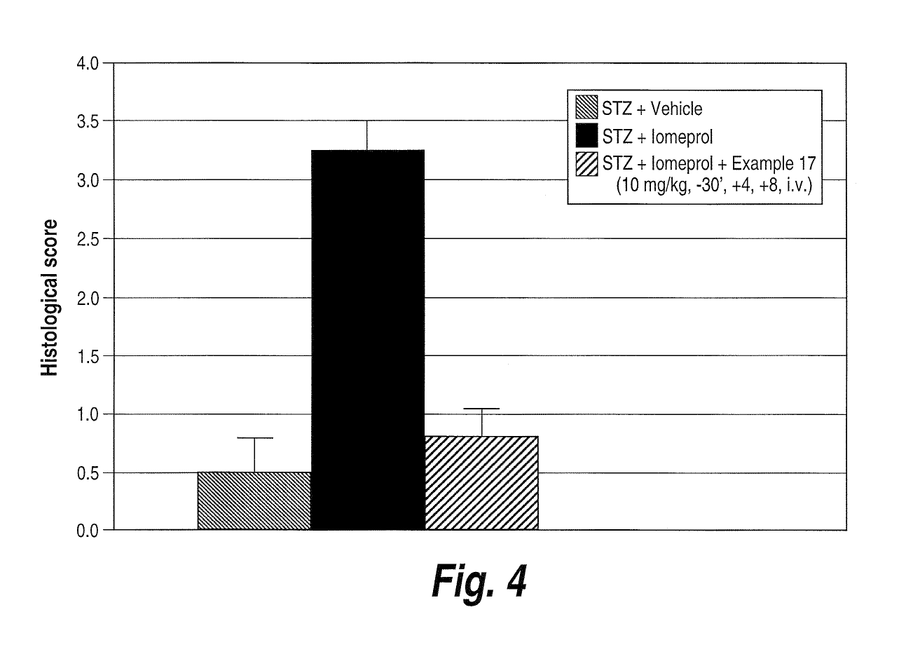

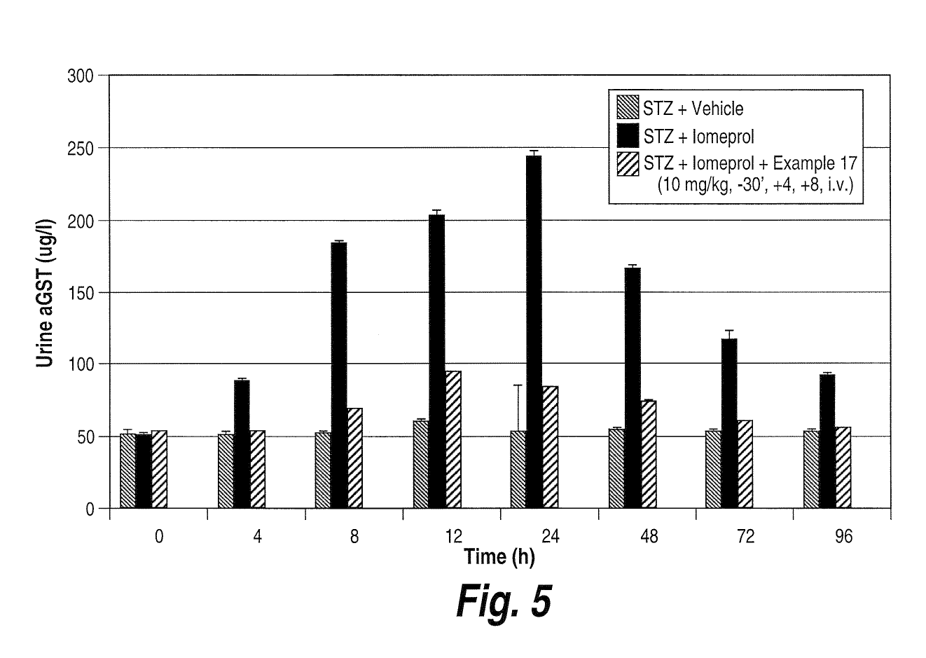

The results are shown graphically in FIGS. 1 to 5. It can be seen from the Figures that administration of PARP inhibitor of Example 17 reduces the level of plasma creatinine, and NGAL, urine NGAL and GST relative to the levels seen with contrast agent alone. The histological scores are also seen to be reduced.

### Example VIII

### Effect of Tetracyclic 1H-indeno[1,2-b]pyridine-2(511)-one Analogs on PARP Activity in Cultured Macrophages, Using a Whole-Cell Based Assay

Demonstration of Tetracyclic 1H-indeno[1,2-b]pyridine-2(5H)-one analogs PARP and prevent peroxynitrite induced cytotoxicity was shown using methods described in Virag et al., Br. J. Pharmacol. 1999, 126(3):769-77; and Immunology 1998, 94(3):345-55. The murine RAW macrophages (ATCC, American Type Culture Collection, Manassas, Va.) were grown in RPMI 1650 (Invitrogen Life Technologies; Carlsbad, Calif.) medium supplemented with 10% heat-inactivated FBS, 2 mM L-glutamine, and 100 U/ml of penicillin and streptomycin. Cells, between passages 5 and 15, were seeded at a density of 250,000 cells/well in 12 well plates and allowed to grow 48 hours before use. Growth medium was changed on the day of use. Cells were treated with a tetracyclic 1H-indeno[1,2-b]pyridine-2(5H)-one analog diluted in a growth medium supplemented with 10% fetal bovine serum (FBS) for 1 hour prior to the addition of hydrogen peroxide (0.5 mM) for a further 25 minutes. For the measurement of PARP activity, media was removed and replaced with 0.5 ml of the assay buffer (56 mM HEPES-pH 7.5, 28 mM KCl, 28 mM NaCl, 2 mM MgCl₂, 0.01% digitonin, and 0.5 μCi/ml of ³H-NAD⁺) for 20 minutes. After aspirating the assay buffer, cells were lysed and transferred to eppendorf tubes containing 250 μl of 50% ice-cold trichloroacetic acid (TCA), which were then placed at 4° C. for 4 hours. Samples were centrifuged at 10,000 g for 10 minutes and supernatant removed. The pellets were washed twice with 500 μl of ice-cold 5% w/v TCA. The pellets were then solubilized in 250 μl of NaOH (0.1 M) containing 2% SDS overnight at 37° C. and the PARP activity was then determined by measuring the radioactivity incorporated using a Wallac scintillation counter. The solubilized protein (250 μl) was mixed with 5 ml of scintillation fluid (ScintiSafe Plus, Fisher Scientific) before being counted for 3 minutes. EC₅₀ values were determined from a dose-response curve.

### Example IX

### Effect of Tetracyclic 1H-indeno[1,2-b]pyridine-2(5H)-one analogs on PARP Activity in purified PARP-1 enzyme assay

The potency of inhibition on purified PARP-1 enzyme was subsequently determined for selected compounds, and the potency was compared with INO-1001. The assay was performed in 96 well ELISA plates according to instructions provided with a commercially available PARP-1 inhibition assay kit (Trevigen, Gaithersburg, Md.). Briefly, wells were coated with 1 mg/ml histone (50 μl/well) at 4° C. overnight. Plates were then washed four times with PBS and then blocked by adding 50 μl Strep-Diluent (supplied with the kit). After incubation (1 h, 25° C.), the plates were washed four times with PBS. Appropriate dilutions of compound was combined with 2×PARP cocktail (1.95 mM NAD⁺, 50 μM biotinylated NAD⁺ in 50 mM TRIS pH 8.0, 25 mM MgCl₂) and high specific activity PARP enzyme (both were supplied with the kit) in a volume of 50 μl. The reaction was allowed to proceed for 30 min at room temperature. After 4 washes in PBS, incorporated biotin was detected by peroxidase-conjugated streptavidin (1:500 dilution) and TACS Sapphire substrate. EC₅₀ and IC₅₀ values were determined from a dose-response curve.

### Biological Data

| Example | EC₅₀ (nM) | IC₅₀ (nM) |
|---|---|---|
| 1 | 11.34 | 3.83 |
| 2 | 1.52 | 5.15 |
| 3 | NT | 11.3 |
| 4 | 1.5 | NT |
| 5 | 7.8 | NT |
| 6 | 8.46 | NT |
| 7 | 7.8 | NT |
| 8 | 8.5 | NT |
| 9 | 12.66 | 93.9 |
| 10 | 37.0 | NT |
| 11 | NT | NT |
| 12 | 748.7 | NT |
| 13 | 169.3 | NT |
| 14 | 12.7 | NT |
| 15 | 11.7 | NT |
| 16 | NT | NT |
| 17 | NT | NT |
| 18 | NT | NT |
| 19 | NT | NT |
| 20 | 4.1 | 5.2 |
| 21 | NT | NT |
| 22 | 7.8 | NT |
| 23 | 20.9 | 12.7 |
| 24 | NT | NT |
| 25 | NT | NT |
| 26 | 5 | 3.4 |
| 27 | 16 | NT |
| 28 | 35 | NT |
| 29 | 25 | NT |
| 30 | 250 | NT |
| 31 | 80 | NT |
| 32 | 100 | 23.1 |
| 33 | 95 | NT |
| 34 | 200 | NT |
| 35 | 300 | NT |
| 36 | 50 | 8.9 |
| 37 | 300 | NT |
| 38 | 300 | NT |
| 39 | 100 | NT |
| 40 | 60 | NT |
| 41 | 45 | 6.3 |
| 42 | 5 | 8.9 |
| 43 | 9 | 8.1 |
| 44 | 200 | NT |
| 45 | 25 | 5.5 |
| 46 | 8 | NT |
| 47 | 9 | 250 |
| 48 | 10 | 27 |
| 49 | 22.67 | 55.7 |
| 50 | 5 | 6.3 |
| 51 | 59 | >250 |
| 52 | 55 | 73.6 |
| 53 | 6 | 89.8 |
| 54 | >500 | NT |
| 55 | 34 | 41.2 |
| 56 | 9 | 19.9 |
| 57 | 334 | NT |
| 58 | 6.1 | 25.5 |
| 59 | 2.4 | 12.4 |
| 60 | 37 | 19.2 |
| 61 | 46 | 27.9 |
| 62 | 13 | 28.1 |
| 63 | 100 | NT |
| 64 | 4.48 | NT |
| 65 | 748 | NT |
| 66 | 37.47 | NT |
| 67 | NT | NT |
| 68 | NT | NT |
| 69 | 18.39 | 54.74 |
| 70 | NT | NT |

*NT = not tested

The present invention is not to be limited in scope by the specific embodiments disclosed in the examples, which are intended as illustrations of a few aspects of the invention and any embodiments that are functionally equivalent are within the scope of this invention. Indeed, various modifications of the invention in addition to those shown and described herein will become apparant to those skilled in the art and are intended to fall within the scope of the appended claims.

A number of references have been cited, the entire disclosures of which have been incorporated herein in their entirety.

1. A compound of Formula (I) 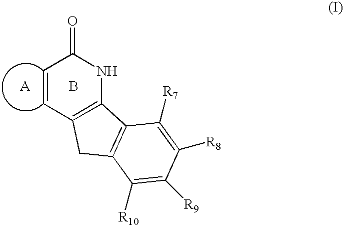
or a pharmaceutically acceptable salt thereof,
wherein
A is a 4-7 membered monocyclic heterocycle ring or a —C₃-C₈ monocyclic ring,
each R₇, R₈, R₉ and R₁₀ is independently —H, -halo, —OH, —CN, —C₁-C₆ alkyl, —C₃-C₈ monocyclic cycloalkyl, —N(R^{a})₂, —C(O)O—C₁-C₆ alkyl, —(C₁-C₆ alkylene)-N(R^{a})₂, —C₁-C₆ alkyl, —N(Z₃)(Z₄), a nitrogen-containing 3- to 8-membered monocyclic heterocycle, —(C₁-C₆ alkylene)-N(Z₃)(Z₄), —(C₁-C₅ alkylene)-(a nitrogen-containing 3- to 8-membered monocyclic heterocycle), S(O)₂—C₁-C₆ alkyl or S(O)₂NH—C₁-C₆ alkyl; each of which other than —H, -halo, and —CN, is independently unsubstituted or substituted with one or more of -halo, —OH, —N(R^{a})₂, —CF₃, —C₁-C₆ alkyl, O—C₁-C₆ alkyl, —C₄-C₆ aryl, —C₄-C₆ aryl substituted with one or more halo, —C₁-C₆ alkylene-C₄-C₆ aryl, —C₁-C₆ alkylene-O—C₁-C₆ alkyl, —C₁-C₆ alkylene-(O—C₁-C₆ alkyl)₂, —C₁-C₆ alkylene-OH, —C₁-C₆ alkylene-N(R^{a})₂, —C₁-C₆ alkylene-C(O)—O—C₁-C₆ alkyl, —C(O)—O—C₁-C₆ alkyl, —C(O)—C₁-C₆ alkylene-OH, —C(O)—N(R^{a})₂, —C(O)—C₁-C₆ alkylene-N(R^{a})₂, a 3- to 7-membered monocyclic heterocycle, or —N(Z₃)(Z₄),
wherein each occurrence of R^{a} is independently —H, halo, benzyl, —C₁-C₁₀ alkyl, —C₁-C₁₀ alkyl-O—C₁-C₁₀ alkyl, C₁-C₁₀ alkyl-OH, —C₁-C₆ alkyl-3-8 membered monocyclic heterocycle, —C₃-C₈ monocyclic alkyl, —C₃-C₈ monocyclic alkyl substituted with one or more of —OH, —C₁-C₆ alkyl, —C₁-C₆ alkylene-NH₂, —O—C₁-C₆ alkylene or —C₁-C₆ alkylene-OH; a 3- to 8-membered monocyclic heterocycle, a 3- to 8-membered monocyclic heterocycle substituted with one or more of —OH, —C₁-C₆ alkyl or —C₁-C₆ alkylene-OH, —(C₁-C₆ alkylene)-O—C₁-C₆ alkyl, C(O)—(C₁-C₆ alkylene)-N—(C₁-C₆ alkyl)₂, —(C₁-C₆ alkylene)-(a 3- to 8-membered monocyclic heterocycle), —C(O)—C₁-C₆ alkylene-S(O)—C₁-C₆ alkyl, —C(O)—C₁-C₆ alkylene-S—C₁-C₆ alkyl, —C(O)—C₁-C₆ alkyl optionally substituted with NH₂ or OH;
wherein N, Z₃ and Z₄ are taken together to form a 3- to 8-membered monocyclic heterocycle which is unsubstituted or substituted with one or more of halo, —OH, —CF₃, —C₁-C₆ alkyl, —NH₂, —NH(C₁-C₆ alkyl), —N(C₁-C₆ alkyl)₂, —C₁-C₆ alkylene-OH, —C₁-C₆ alkylene-halo, —C₁-C₆ alkylene-NH₂, —C₃-C₈ monocyclic alkyl, a 3- to 8-membered monocyclic heterocycle, —C₁-C₆ alkylene-C₃-Cₛ-monocyclic alkyl, —C(O)C₃-C₈ monocyclic alkyl, —C(O)-3-8 membered monocyclic heterocycle, —C(O)—NH₂, —C(O)—(C₁-C₆ alkylene)-NH—C₁-C₆ alkyl; C(O)—(C₁-C₆ alkylene)-N—(C₁-C₆ alkyl)₂; C(O)—C₁-C₆ alkyl optionally substituted with NH₂ or OH, —C₁-C₆ alkylene-O—C₁-C₆ alkyl or —O—C₁-C₆ alkyl.

2. A compound of Formula (I) as defined in claim 1 wherein A is selected from 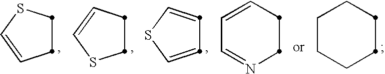
where represents the points of attachment to the B ring.

3. A compound of Formula (I) as defined in claim 2 wherein A is

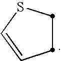

4. A compound of Formula (I) or a pharmaceutically acceptable salt thereof as defined in claim 2 wherein A is

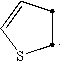

5. A compound of Formula (I) or a pharmaceutically acceptable salt thereof as defined in claim 2 wherein A is

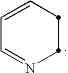

6. A compound of Formula (I) or a pharmaceutically acceptable salt thereof as defined in claim 2 wherein A is

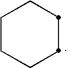

7. A compound of Formula (I) or a pharmaceutically acceptable salt thereof as defined in claim 1 wherein R₇ and R₈ are —H and R₉ and R₁₀ are independently selected from —H, -halo, —OH, —CN, —C₁-C₆ alkyl, —C₃-C₈ monocyclic cycloalkyl, —N(R^{a})₂, —C(O)O—C₁-C₆ alkyl, —(C₁-C₆ alkylene)-N(R^{a})₂, —C₁-C₆ alkyl, —N(Z₃)(Z₄), a nitrogen-containing 3- to 8-membered monocyclic heterocycle, —(C₁-C₆ alkylene)-N(Z₃)(Z₄), —(C₁-C₅ alkylene)-(a nitrogen-containing 3- to 8-membered monocyclic heterocycle), S(O)₂—C₁-C₆ alkyl or S(O)₂NH—C₁-C₆ alkyl;
each of which other than —H, -halo, and —CN, is independently unsubstituted or substituted with one or more of -halo, —OH, —N(R^{a})₂, —CF₃, —C₁-C₆ alkyl, O—C₁-C₆ alkyl, —C₄-C₆ aryl, —C₄-C₆ aryl substituted with one or more halo, —C₁-C₆ alkylene-C₄-C₆ aryl, —C₁-C₆ alkylene-O—C₁-C₆ alkyl, —C₁-C₆ alkylene-(O—C₁-C₆ alkyl)₂, —C₁-C₆ alkylene-OH, —C₁-C₆ alkylene-N(R^{a})₂, —C₁-C₆ alkylene-C(O)—O—C₁-C₆ alkyl, —C(O)—O—C₁-C₆ alkyl, —C(O)—C₁-C₆ alkylene-OH, —C(O)—N(R^{a})₂, —C(O)—C₁-C₆ alkylene-N(R^{a})₂, a 3- to 7-membered monocyclic heterocycle, or —N(Z₃)(Z₄),
wherein each occurrence of R^{a} is independently —H, halo, benzyl, —C₁-C₁₀ alkyl, —C₁-C₁₀ alkyl-O—C₁-C₁₀ alkyl, C₁-C₁₀ alkyl-OH, —C₁-C₆ alkyl-3-8 membered monocyclic heterocycle, —C₃-C₈ monocyclic alkyl, —C₃-C₈ monocyclic alkyl substituted with one or more of —OH, —C₁-C₆ alkyl, —C₁-C₆ alkylene-NH₂, —O—C₁-C₆ alkylene or —C₁-C₆ alkylene-OH; a 3- to 8-membered monocyclic heterocycle, a 3- to 8-membered monocyclic heterocycle substituted with one or more of —OH, —C₁-C₆ alkyl or —C₁-C₆ alkylene-OH, —(C₁-C₆ alkylene)-O—C₁-C₆ alkyl, C(O)—(C₁-C₆ alkylene)-N—(C₁-C₆ alkyl)₂, —(C₁-C₆ alkylene)-(a 3- to 8-membered monocyclic heterocycle), —C(O)—C₁-C₆ alkylene-S(O)—C₁-C₆ alkyl, —C(O)—C₁-C₆ alkylene-S—C₁-C₆ alkyl, —C(O)—C₁-C₆ alkyl optionally substituted with NH₂ or OH;
wherein N, Z₃ and Z₄ are taken together to form a 3- to 8-membered monocyclic heterocycle which is unsubstituted or substituted with one or more of halo, —OH, —CF₃, —C₁-C₆ alkyl, —NH₂, —NH(C₁-C₆ alkyl), —N(C₁-C₆ alkyl)₂, —C₁-C₆ alkylene-OH, —C₁-C₆ alkylene-halo, —C₁-C₆ alkylene-NH₂, —C₃-C₈ monocyclic alkyl, a 3- to 8-membered monocyclic heterocycle, —C₁-C₆ alkylene-C₃-C₈-monocyclic alkyl,
—C(O)C₃-C₈ monocyclic alkyl, —C(O)-3-8 membered monocyclic heterocycle, —C(O)—NH₂, —C(O)—(C₁-C₆ alkylene)-NH—C₁-C₆ alkyl; C(O)—(C₁-C₆ alkylene)-N—(C₁-C₆ alkyl)₂; C(O)—C₁-C₆ alkyl optionally substituted with NH₂ or OH, —C₁-C₆ alkylene-O—C₁-C₆ alkyl or —O—C₁-C₆ alkyl.

8. A compound of Formula (I) or a pharmaceutically acceptable salt thereof as defined in claim 7 wherein A is

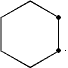

9. A compound of Formula (I) or a pharmaceutically acceptable salt thereof as defined in claim 8 wherein R₉ or R₁₀ is selected from

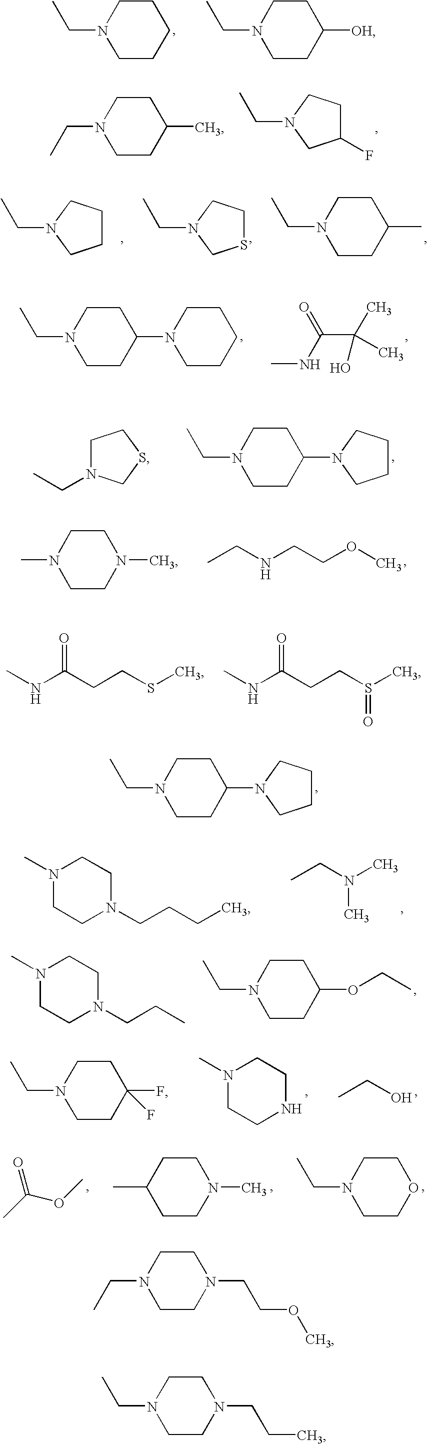

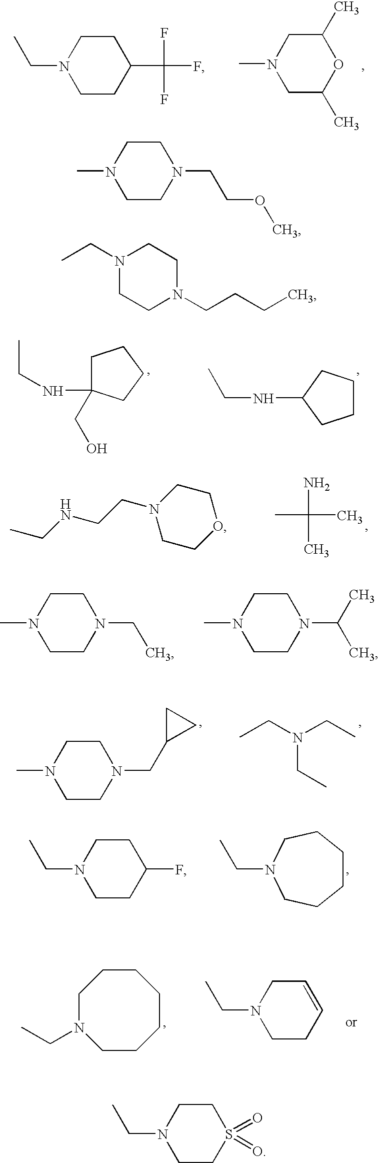

10. A compound of Formula (I) or a pharmaceutically acceptable salt hereof as defined in claim 2 wherein A is 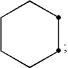
each R₇, R₈, and R₉ are H; and
R₁₀ is —(C₁-C₃ alkylene)-N(Z₃)(Z₄);
wherein N, Z₃ and Z₄ are taken together to form piperidine, azepane, or pyrrolidine, wherein the piperidine, azepane, or pyrrolidine rings are independently substituted with halo or —C₁-C₃ alkyl.

11. A compound of Formula (I) or a pharmaceutically acceptable salt thereof as defined in claim 1 selected from:
10-((4-hydroxypiperidin-1-yl)methyl)-3,4,6,11-tetrahydro-1H-indeno[1,2-c]isoquinolin-5(2H)-one,
10-(piperidin-1-ylmethyl)-3,4,6,11-tetrahydro-1H-indeno[1,2-c]isoquinolin-5(2H)-one,
10-(azepan-1-ylmethyl)-3,4,6,11-tetrahydro-1H-indeno[1,2-c]isoquinolin-5(2H)-one,
10-(pyrrolidin-1-ylmethyl)-3,4,6,11-tetrahydro-1H-indeno[1,2-c]isoquinolin-5(2H)-one,
10-((4-ethoxypiperidin-1-yl)methyl)-3,4,6,11-tetrahydro-1H-indeno[1,2-c]isoquinolin-5(2H)-one,
10-(azocan-1-ylmethyl)-3,4,6,11-tetrahydro-1H-indeno[1,2-c]isoquinolin-5(2H)-one,
10-((4,4-difluoropiperidin-1-yl)methyl)-3,4,6,11-tetrahydro-1H-indeno[1,2-c]isoquinolin-5(2H)-one,
10-(thiazolidin-3-ylmethyl)-3,4,6,11-tetrahydro-1H-indeno[1,2-c]isoquinolin-5(2H)-one,
(S)-10-((3-fluoropyrrolidin-1-yl)methyl)-3,4,6,11-tetrahydro-1H-indeno[1,2-c]isoquinolin-5(2H)-one,
(R)-10-((3-fluoropyrrolidin-1-yl)methyl)-3,4,6,11-tetrahydro-1H-indeno[1,2-c]isoquinolin-5(2H)-one,
10-((4-methylpiperidin-1-yl)methyl)-3,4,6,11-tetrahydro-1H-indeno[1,2-c]isoquinolin-5(2H)-one,
10-(1,4′-bipiperidin-1′-ylmethyl)-3,4,6,11-tetrahydro-1H-indeno[1,2-c]isoquinolin-5(2H)-one,
10-((4-(pyrrolidin-1-yl)piperidin-1-yl)methyl)-3,4,6,11-tetrahydro-1H-indeno[1,2-c]isoquinolin-5(2H)-one,
10-((cyclopentylamino)methyl)-3,4,6,11-tetrahydro-1H-indeno[1,2-c]isoquinolin-5(2H)-one,
10-((2-methoxyethylamino)methyl)-3,4,6,11-tetrahydro-1H-indeno[1,2-c]isoquinolin-5(2H)-one,
10-((dimethylamino)methyl)-3,4,6,11-tetrahydro-1H-indeno[1,2-c]isoquinolin-5(2H)-one),
10-(hydroxy methyl)-3,4,6,11-tetrahydro-1H-indeno[1,2-c]isoquinolin-5 (2H)-one,
methyl 5-oxo-2,3,4,5,6,11-hexahydro-1H-indeno[1,2-c]isoquinoline-9-carboxylate,
10-(piperazin-1-ylmethyl)-3,4,6,11-tetrahydro-1H-indeno[1,2-c]isoquinolin-5(2H)-one,
10-(2,6-dimethylmorpholino)-3,4,6,1,1-tetrahydro-1H-indeno[1,2-c]isoquinolin-5(2H)-one,
9-(hydroxymethyl)-3,4,6,11-tetrahydro-1H-indeno[1,2-c]isoquinolin-5(2H)-one,
9-((4-propylpiperazin-1-yl)methyl)-3,4,6,11-tetrahydro-1H-indeno[1,2-c]isoquinolin-5(2H)-one,
9-((4-(2-methoxyethyl)piperazin-1-yl)methyl)-3,4,6,11-tetrahydro-1H-indeno[1,2-c]isoquinolin-5(2H)-one,
9-((2-methoxyethylamino)methyl)-3,4,6,11-tetrahydro-1H-indeno[1,2-c]isoquinolin-5(2H)-one,
9-(morpholinomethyl)-3,4,6,11-tetrahydro-1H-indeno[1,2-c]isoquinolin-5(2H)-one,
9-((4-hydroxypiperidin-1-yl)methyl)-3,4,6,11-tetrahydro-1H-indeno[1,2-c]isoquinolin-5(2H)-one,
9-(pyrrolidin-1-ylmethyl)-3,4,6,11-tetrahydro-1H-indeno[1,2-c]isoquinol in -5(2H)-one,
9-((cyclopentylamino)methyl)-3,4,6,11-tetrahydro-1H-indeno[1,2-c]isoquinolin-5(2H)-one,
10-(4-propylpiperazin-1-yl)-3,4,6,11-tetrahydro-1H-indeno[1,2-c]isoquinolin-5 (2H)-one,
9-(4-butylpiperazin-1-yl)-3,4,6,11-tetrahydro-1H-indeno[1,2-c]isoquinolin-5(2H)-one,
10-(4-(2-methoxyethyl)piperazin-1-yl)-3,4,6,11-tetrahydro-1H-indeno[1,2-c]isoquinolin-5(2H)-one,
9-((4-butylpiperazin-1-yl)methyl)-3,4,6,11-tetrahydro-1H-indeno[1,2-c]isoquinolin-5(2H)-one,
9-((2-morpholinoethylamino)methyl)-3,4,6,11-tetrahydro-1H-indeno[1,2-c]isoquinolin-5(2H)-one,
10-((1-(hydroxymethyl)cyclopentylamino)methyl)-3,4,6,11-tetrahydro-1H-indeno[1,2-c]isoquinolin-5(2H)-one,
2-hydroxy-2-methyl-N-(5-oxo-2,3,4,5,6,11-hexahydro-1H-indeno[1,2-c]isoquinolin-10-yl)propanamide,
3-(methylthio)-N-(5-oxo-2,3,4,5,6,11-hexahydro-1H-indeno[1,2-c]isoquinolin-10-yl)propanamide,
3-(methylsulfinyl)-N-(5-oxo-2,3,4,5,6,11-hexahydro-1H-indeno[1,2-c]isoquinolin-10-yl)propanamide,
methyl 5-oxo-2,3,4,5,6,11-hexahydro-1H-indeno[1,2-c]isoquinoline-10-carboxylate,
10-(2-aminopropan-2-yl)-3,4,6,11-tetrahydro-1H-indeno[1,2-c]isoquinolin-5(2H)-one,
9-((diethylamino)methyl)-3,4,6,11-tetrahydro-1H-indeno[1,2-c]isoquinolin-5(2H)-one,
9-((4,4-difluoropiperidin-1-yl)methyl)-3,4,6,11-tetrahydro-1H-indeno[1,2-c]isoquinolin-5(2H)-one,
9-((4-fluoropiperidin-1-yl)methyl)-3,4,6,11-tetrahydro-1H-indeno[1,2-c]isoquinolin-5(2H)-one,
10-((diethylamino)methyl)-3,4,6,11-tetrahydro-1H-indeno[1,2-c]isoquinolin-5(2H)-one,
10-((4-fluoropiperidin-1-yl)methyl)-3,4,6,11-tetrahydro-1H-indeno[1,2-c]isoquinolin-5(2H)-one,
10-((5,6-dihydropyridin-1(2H)-yl)methyl)-3,4,6,11-tetrahydro-1H-indeno[1,2-c]isoquinolin-5(2H)-one, and
10-((thiomorpholinomethyl-1,1-dioxide)-3,4,6,11-tetrahydro-1H-indeno[1,2-c]isoquinolin-5(2H)-one.

12. The compound of claim 1 being 10-(azepan-1-ylmethyl)-3,4,6,11-tetrahydro-1H-indeno[1,2-c]isoquinolin-5(2H)-one or a pharmaceutically acceptable salt thereof.

13. The compound of claim 1 being 10-(piperidin-1-ylmethyl)-3,4,6,11-tetrahydro-1H-indeno[1,2-c]isoquinolin-5(2H)-one or a pharmaceutically acceptable salt thereof.

14. The compound of claim 1 being 10-((4-methylpiperidin-1-yl)methyl)-3,4,6,11-tetrahydro-1H-indeno[1,2-c]isoquinolin-5(2H)-one or a pharmaceutically acceptable salt thereof.

15. The compound of claim 1 being (S)-10-((3-fluoropyrrolidin-1-yl)methyl)-3,4,6,11-tetrahydro-1H-indeno[1,2-c]isoquinolin-5(2H)-one or a pharmaceutically acceptable salt thereof.

16. A composition comprising a physiologically acceptable carrier or vehicle and an effective amount of a compound or a pharmaceutically acceptable salt of the compound of claim 1.

17. A method for treating cancer, comprising administering an effective amount of a compound or a pharmaceutically acceptable salt of the compound of claim 1 to a subject in need thereof.

18. The method of claim 17, further comprising administering an effective amount of another anticancer agent.

19. A method for treating renal failure, comprising administering an effective amount of a compound or a pharmaceutically acceptable salt of the compound of claim 1 to a subject in need thereof.

20. A method for treating a reperfusion injury, comprising administering an effective amount of a compound or a pharmaceutically acceptable salt of the compound of claim 1 to a subject in need thereof.

21. A method for treating an inflammatory disease, comprising administering an effective amount of a compound or a pharmaceutically acceptable salt of the compound of claim 1 to a subject in need thereof.

22. A method for treating diabetes mellitus, comprising administering an effective amount of a compound or a pharmaceutically acceptable salt of the compound of claim 1 to a subject in need thereof.

23. A method for treating a retinal or an optic nerve disease or damage related to or from glaucoma, which comprises administering to a mammal a composition containing an effective amount of one or more PARD inhibitors in a pharmaceutically acceptable vehicle.

24. (canceled)

25. The method of claim 23 wherein the one or more PARP inhibitors are selected from a compound or a pharmaceutically acceptable salt of the compound of claim 1 to a subject in need thereof.

26. A method for treating an ischemic condition, comprising administering an effective amount of a compound or a pharmaceutically acceptable salt of the compound of claim 1 to a subject in need thereof.

27. A method for treating reoxygenation injury resulting from organ transplantation, comprising administering an effective amount of a compound or a pharmaceutically acceptable salt of the compound of claim 1 to a subject in need thereof.

28. A method for treating a neurodegenerative disease, comprising administering an effective amount of a compound or a pharmaceutically acceptable salt of the compound of claim 1 to a subject in need thereof.

29. A method for treating a vascular disease, comprising administering an effective amount of a compound or a pharmaceutically acceptable salt of the compound of claim 1 to a subject in need thereof.

30. A method for treating a diabetic complication, comprising administering an effective amount of a compound or a pharmaceutically acceptable salt of the compound of claim 1 to a subject in need thereof.

31. A method for treating a cardiovascular disease, comprising administering an effective amount of a compound or a pharmaceutically acceptable salt of the compound of claim 1 to a subject in need thereof.

32. A method for treating erectile dysfunction, comprising administering an effective amount of a compound or a pharmaceutically acceptable salt of the compound of claim 1 to a subject in need thereof.

33. A method for treating urinary incontinence, comprising administering an effective amount of a compound or a pharmaceutically acceptable salt of the compound of claim 1 to a subject in need thereof.

34. A method for treating a complication of prematurity, comprising administering an effective amount of a compound or a pharmaceutically acceptable salt of the compound of claim 1 to a subject in need thereof.

35. A method for treating cardiomyopathy, comprising administering an effective amount of a compound or a pharmaceutically acceptable salt of the compound of claim 1 to a subject in need thereof.

36. A method for treating retinopathy, comprising administering an effective amount of a compound or a pharmaceutically acceptable salt of the compound of claim 1 to a subject in need thereof.

37. A method for treating nephropathy, comprising administering an effective amount of a compound or a pharmaceutically acceptable salt of the compound of claim 1 to a subject in need thereof.

38. A method for prevention of contrast induced nephropathy, comprising administering an effective amount of a compound or a pharmaceutically acceptable salt of the compound of claim 1 to a subject in need thereof prior to administration of a contrast-agent.

39. A method for prevention of contrast induced nephropathy, comprising administering an effective amount of a compound or a pharmaceutically acceptable salt of the compound of claim 1 and an antioxidative agent to a subject in need thereof prior to administration of a contrast-agent.

40. (canceled)

41. A method for treating neuropathy, comprising administering an effective amount of a compound or a pharmaceutically acceptable salt of the compound of claim 1 to a subject in need thereof.

42. A method for treating an ocular disease or condition comprising administering an effective amount of a compound or a pharmaceutically acceptable salt of the compound of claim 1 to a subject in need thereof.

43. A method for treating aged macular degeneration comprising administering an effective amount of a compound or a pharmaceutically acceptable salt of the compound of claim 1 to a subject in need thereof.

44. A method for treating a retinopathy, including diabetic retinopathy, hypertensive retinopathy and retinopathy of prematurity comprising administering an effective amount of a compound or a pharmaceutically acceptable salt of the compound of claim 1 to a subject in need thereof.

45. A method for treating glaucoma or a glaucoma related condition, comprising administering an effective amount of a compound or a pharmaceutically acceptable salt of the compound of claim 1 to a subject in need thereof.

46. A method for treating ocular angiogenesis comprising administering an effective amount of a compound or a pharmaceutically acceptable salt of the compound of claim 1 to a subject in need thereof.

47. A method for treating an ocular neovascular disease or condition comprising administering an effective amount of a compound or a pharmaceutically acceptable salt of the compound of claim 1 to a subject in need thereof.
